(19) United States
(12) Patent Application Publication
(10) Pub. No.: US 2024/0209109 A1
(43) Pub. Date: Jun. 27, 2024

(54) TUMOR ANTIGEN-DEPENDENT CD40 AGONISTS ANTIBODIES

(71) Applicants: CONCEPT TO MEDICINE BIOTECH CO., LTD., Shanghai (CN); LEPU BIOPHARMA CO., LTD., Shanghai (CN)

(72) Inventors: Fang LIU, Shanghai (CN); Yuanyuan YANG, Shanghai (CN); Wenci GONG, Shanghai (CN); Zhijian CAI, Shanghai (CN); Wenqing JIANG, Shanghai (CN); Lei FANG, Shanghai (CN)

(21) Appl. No.: 18/582,578

(22) Filed: Feb. 20, 2024

Related U.S. Application Data
(63) Continuation of application No. PCT/CN2023/077495, filed on Feb. 21, 2023.

(30) Foreign Application Priority Data
Jan. 21, 2022 (WO) PCT/CN2022/077029
Jul. 28, 2022 (WO) PCT/CN2022/108746

(51) Int. Cl. C07K 16/28; A61K 39/00; A61K 45/06; A61P 35/00

(52) U.S. Cl. CPC C07K 16/2878; A61K 45/06; A61P 35/00; A61K 2039/505; C07K 2317/31; C07K 2317/565; C07K 2317/569

(57) ABSTRACT

Provided are CD40 agonist antibodies which have greatly higher CD40 activation activity in the presence of concurrent tumor-associated antigen (TAA) binding than without such concurrent binding. Such TAA-dependent CD40 agonism results in greatly improved therapeutic index, reducing or totally eliminating adverse events commonly associated with CD40 activation, such as cytokine release syndrome or liver toxicity. Also provided are bi- and multi-specific antibodies and polypeptides, such as chimeric antigen receptors, that incorporate these antibodies, optionally with an anti-TAA unit. Methods of using the antibodies or polypeptides for treating and diagnosing diseases such as cancer are also provided.

### CROSS REFERENCE TO RELATED APPLICATIONS

The present application is a continuation of International Patent Application No. PCT/CN2023/077495 filed Feb. 21, 2023, which claims the priority to PCT/CN2022/108746, filed Jul. 28, 2022, and PCT/CN2022/077029, filed Feb. 21, 2022, each of which is incorporated herein by reference in its entirety.

### REFERENCE TO AN ELECTRONIC SEQUENCE LISTING

The content of the electronic sequence listing (344056US.xml; Size: 155,549 bytes; and Date of Creation: Feb. 20, 2024) is herein incorporated by reference in its entirety.

### BACKGROUND

CD40 (tumor necrosis factor receptor superfamily 5, or cluster of differentiation 40) is a member of TNF-receptor superfamily (TNFRSF) and is a costimulatory molecule which is expressed on antigen present cells (APCs), such as dendritic cell (DC), B cell and macrophage, as well as on non-immune cells and tumors. CD40 plays an important role in modulating the activity of APCs and connecting innate and adaptive immunity. The CD40 pathway is not only required for effective T- and B-cell immune responses but also provides a critical initial step in the development of humoral and cellular immunity.

Upon activation, CD40 can induce dendritic cells to promote antitumor T cell activation and re-educate macrophages to destroy tumor stroma. The activation of CD40 has been used in combination with other therapies for cancer treatments, such as immune checkpoint inhibitors. The combination of a chemotherapy followed by CD40 activation functions as an in situ vaccine. Moreover, it was reported that CD40-activated macrophages rapidly infiltrated tumors and facilitated the depletion of tumor stroma and further enhance chemotherapy delivery. Taken together, CD40 activation contributes to an important mechanism to convert so-called cold tumors to hot ones.

Multiple approaches have been formulated to activate CD40 in patients with cancer. The initial CD40 therapeutic agonists were based on multimeric versions of its ligand, CD40L. Subsequently, the approaches largely were based on agonist CD40 antibodies, designed to mimic CD40L by crosslinking CD40. The most extensively studied one is selicrelumab (Roche), formerly known as CP-870,893 and RO7009789, which is a fully human IgG2 mAb. Others include CDX-1140 (Celldex), APX005M (Apexigen), SEA-CD40 (Seattle Genetics), ChiLob7/4 (University of Southampton), and ADC-1013 (Janssen/Alligator). CD40 antibodies vary with regard to activation potency, ranging from very high (APX005M), high (selicrelumab), to weak (SEA-CD40). Some CD40 mAbs block the CD40L binding site, such as APX005M), while others (e.g., selicrelumab and CDX-1140) do not.

Clinical studies of these CD40 agonists revealed a common set of adverse events that are dose dependent. Among them, the main adverse event is the cytokine release syndrome (CRS) as characterized by a variety of combinations of chills, rigors, rash, nausea, fever, vomiting, muscle aches and back pain. Another major safety issue is dose-related haematological toxicities, such as a decrease in peripheral lymphocytes, monocytes and platelets. Such reported toxicities are seen as inherit to CD40 agonist therapies and have dissuaded experts from advancing clinical development of these candidates. There is a strong need to develop CD40 agonist antibodies that have potent therapeutic effects without the associated toxicities.

### SUMMARY

As provided, the existing CD40 agonist antibodies, although having acceptable CD40 activation efficacy, are associated with common adverse events, such as cytokine release syndrome (CRS). The adverse effects are inherently associated with the biological mechanism of CD40 agonism and thus it is challenging to control the adverse effects without sacrificing therapeutic efficacy.

Through careful design and selection, however, the instant inventors have identified new CD40 agonist nanobodies that have considerably reduced CD40 activation capability as compared to the existing antibodies, such as selicrelumab. When used in a bi- or multi-specific format that further includes an antibody portion that targets a tumor-associated antigen (TAA) expressed on the target cell, however, the newly identified antibodies exhibited potent activation activities. The TAA-dependency of the newly identified antibodies, therefore, enables these new antibodies to be highly therapeutically active where the activity is required (e.g., at a target tumor site) and less active or even inactive elsewhere. The latter property, therefore, can reduce or even eliminate those adverse effects commonly associated with other CD40 agonist antibodies.

In accordance with one embodiment of the present disclosure, therefore, provided is a single domain antibody or a polypeptide comprising the single domain antibody, wherein the single domain antibody has binding specificity to the human cluster of differentiation 40 (CD40) protein and comprises a complementarity determining region 1 (CDR1), a CDR2 and a CDR3.

In some embodiments, the CDR1, CDR2 and CDR3 comprise, respectively, (1) the amino acid sequences of SEQ ID NO: 14, 15 and 16; (1a) the amino acid sequences of SEQ ID NO: 14, 63 and 16; (1b) the amino acid sequences of SEQ ID NO: 14, 64 and 16; (2) the amino acid sequences of SEQ ID NO: 17, 18 and 19; (3) the amino acid sequences of SEQ ID NO: 20, 21 and 22; (4) the amino acid sequences of SEQ ID NO: 23, 24 and 25; (5) the amino acid sequences of SEQ ID NO: 26, 27 and 28; (6) the amino acid sequences of SEQ ID NO: 29, 30 and 31; (7) the amino acid sequences of SEQ ID NO: 32, 33 and 34; (8) the amino acid sequences of SEQ ID NO: 35, 36 and 37; (9) the amino acid sequences of SEQ ID NO: 38, 39 and 40; (10) the amino acid sequences of SEQ ID NO: 41, 42 and 43; (11) the amino acid sequences of SEQ ID NO: 44, 45 and 46; (12) the amino acid sequences of SEQ ID NO: 47, 48 and 49; or (13) the amino acid sequences of SEQ ID NO: 50, 51 and 52.

In some embodiments, the CDR1 comprises the amino acid sequence of SEQ ID NO:14, the CDR2 comprises the amino acid sequence of SEQ ID NO: 15, 63 or 64, and the CDR3 comprises the amino acid sequence of SEQ ID NO:16.

In some embodiments, the CDR1 comprises the amino acid sequence of SEQ ID NO: 14, the CDR2 comprises the amino acid sequence of SEQ ID NO: 15, and the CDR3 comprises the amino acid sequence of SEQ ID NO:16. In some embodiments, the antibody or polypeptide comprises an amino acid sequence selected from the group consisting of SEQ ID NO: 53, 54, 57 and 60.

In some embodiments, the CDR1 comprises the amino acid sequence of SEQ ID NO:14, the CDR2 comprises the amino acid sequence of SEQ ID NO:63, and the CDR3 comprises the amino acid sequence of SEQ ID NO: 16. In some embodiments, the antibody or polypeptide comprises an amino acid sequence selected from the group consisting of SEQ ID NO: 55, 58 and 61.

In some embodiments, the CDR1 comprises the amino acid sequence of SEQ ID NO: 14, the CDR2 comprises the amino acid sequence of SEQ ID NO:64, and the CDR3 comprises the amino acid sequence of SEQ ID NO:16. In some embodiments, the antibody or polypeptide comprises an amino acid sequence selected from the group consisting of SEQ ID NO: 56, 59 and 62.

Also provided, in one embodiment, is a multi-specific antibody comprising the antibody of the present disclosure and a second antibody or antigen-binding fragment having binding specificity to a second target antigen that is not CD40. In some embodiments, the second target antigen is a tumor-associated antigen (TAA).

Another embodiment provides a multi-specific antibody comprising a first antibody or antigen-binding fragment having binding specificity to human CD40, and a second antibody or antigen-binding fragment having binding specificity to a second target antigen that is a tumor-associated antigen (TAA), wherein the multi-specific antibody activates CD40 on a target cell that expresses the TAA more effectively than CD40 on a reference cell that does not express the TAA.

Another embodiment provides a multi-specific antibody comprising a first antibody or antigen-binding fragment having binding specificity to human CD40, and a second antibody or antigen-binding fragment having binding specificity to a second target antigen that is a tumor-associated antigen (TAA), wherein the multi-specific antibody does not activate CD40 on a reference cell that does not express the TAA.

In some embodiments, the multi-specific antibody activates CD40 on the target cell that expresses the TAA at least as 2 times, or 5 times, 10 times, 20 times, 50 times or 100 times, as effective as CD40 on the reference cell that does not express the TAA. In some embodiments, the activation is measured with a concentration of the multi-specific antibody from 0.001 to 200 nM; preferably from 0.1 to 100 nM. In some embodiments, the activation is measured with a panel of CD40 function assays, such as NFκB reporter assay, an IL-12 secretion assay, a CD80 expression assay, a CD86 expression assay or a Ki67 expression assay, or a Ki67/CD86 expression assay.

The instant disclosure provides conditionally activated CD40 bispecific antibodies that are only activated in the presence of 5T4-expressing tumor cells. 5T4 is a oncofetal protein rarely expressed in normal adult tissues, however, the expression is upregulated in multiple cancers. It is contemplated that CD40 crosslinking by engagement of 5T4 on the cancer cells can boost the immune response in the tumor microenvironment while minimizing the risk of peripheral toxicity. Meanwhile, by restricting antibody in the 5T4-expressing tumor cells, it is contemplated, 5T4×CD40 bispecific antibody could overcome the antigen sink due to the broad peripheral CD40 expression, allowing accumulation of the molecule in the tumor.

In accordance with one embodiment of the present disclosure, provided is a multi-specific antibody comprising a first antibody or antigen-binding fragment having binding specificity to a CD40 protein, and a second antibody or antigen-binding fragment having binding specificity to a 5T4 protein, wherein the multi-specific antibody activates CD40 on a target cell that expresses the 5T4 protein more effectively than CD40 on a reference cell that does not express the 5T4 protein, or wherein the multi-specific antibody does not activate CD40 on a reference cell that does not express the 5T4 protein.

In some embodiments, the multi-specific antibody activates CD40 on the target cell that expresses the 5T4 protein at least as 2 times, or 5 times, 10 times, 20 times, 50 times or 100 times, as effective as CD40 on the reference cell that does not express the 5T4 protein. In some embodiments, the activation is measured with a concentration of the multi-specific antibody from 0.001 to 200 nM; preferably from 0.1 to 100 nM. In some embodiments, the activation is measured with a panel of CD40 function assay; preferably, wherein the activation is measured with a NFκB reporter assay, an IL-12 secretion assay, a CD80 expression assay, a CD86 expression assay or a Ki67 expression assay, or a Ki67/CD86 expression assay.

In some embodiments, the first antibody or antigen-binding fragment comprises two or three concatenated single domain (VHH) anti-CD40 antibodies. In some embodiments, the second antibody or antigen-binding fragment comprises a conventional VH/VL Fab fragment. In some embodiments, the two or three concatenated VHH anti-CD40 antibodies and the conventional VH/VL Fab fragment each is fused to the N-terminus of each of the two chains of a Fc fragment.

In some embodiments, the first antibody or antigen-binding fragment comprises two separate (VHH) anti-CD40 antibodies, each is fused to the C-terminus of each of the two chains of a Fc fragment, and wherein the second antibody or antigen-binding fragment comprises two conventional VH/VL Fab fragments, each is fused to the N-terminus of each of the two chains of the Fc fragment.

In some embodiments, the Fc fragment is a human IgG1, IgG2 or IgG4 fragment. In some embodiments, the Fc fragment comprises substitutions L234A, L235A and N297A, L234A and L235A, or N297A, according to Kabat numbering.

In some embodiments, the first antibody or antigen-binding fragment comprises one or more single domain (VHH) anti-CD40 antibodies, each comprises a CDR1 comprising the amino acid sequence of SEQ ID NO: 14, a CDR2 comprising the amino acid sequence of SEQ ID NO: 15, 63 or 64, and a CDR3 comprising the amino acid sequence of SEQ ID NO: 5. In some embodiments, the VHH antibodies each comprises an amino acid sequence selected from the group consisting of SEQ ID NO: 1 and 53-62. In some embodiments, the VHH antibodies each comprises the amino acid sequence of SEQ ID NO: 54.

In some embodiments, the second antibody or antigen-binding fragment competes with antibody 14G12 or 159D5 in binding to the 5T4 protein, wherein the antibody 14G12 comprises a VH of SEQ ID NO: 73 and a VL of SEQ ID NO: 74, and the antibody 159D5 comprises a VH of SEQ ID NO: 121 and a VL of SEQ ID NO: 122.

In some embodiments, the second antibody or antigen-binding fragment comprises a heavy chain variable region (VH) comprising a VH CDR1, VH CDR2, and VH CDR3, and a light chain variable region (VL) comprising a VL CDR1, VL CDR2, and VL CDR3, wherein the VH CDR1, VH CDR2, VH CDR3, VL CDR1, VL CDR2, and VL CDR3, respectively, comprise the amino sequences of SEQ ID NO: 130-80. In some embodiments, the VH comprises an amino acid sequence selected from the group consisting of SEQ ID NO: 73 and 81-90, and the VL comprises an amino acid sequence selected from the group consisting of SEQ ID NO: 74 and 91-100. In some embodiments, the VH comprises the amino acid of SEQ ID NO: 83 or 89, and the VL comprises the amino acid sequence of SEQ ID NO: 91.

In some embodiments, the second antibody or antigen-binding fragment comprises a heavy chain variable region (VH) comprising a VH CDR1, VH CDR2, and VH CDR3, and a light chain variable region (VL) comprising a VL CDR1, VL CDR2, and VL CDR3, wherein the VH CDR1, VH CDR2, VH CDR3, VL CDR1, VL CDR2, and VL CDR3, respectively, comprise the amino sequences of SEQ ID NO: 103-108. In some embodiments, the VH comprises an amino acid sequence selected from the group consisting of SEQ ID NO: 101 and 109-115, and the VL comprises an amino acid sequence selected from the group consisting of SEQ ID NO: 102 and 116-120. In some embodiments, the VH comprises the amino acid of SEQ ID NO: 113, and the VL comprises the amino acid sequence of SEQ ID NO: 120.

In some embodiments, the second antibody or antigen-binding fragment comprises a heavy chain variable region (VH) comprising a VH CDR1, VH CDR2, and VH CDR3, and a light chain variable region (VL) comprising a VL CDR1, VL CDR2, and VL CDR3, wherein the VH CDR1, VH CDR2, VH CDR3, VL CDR1, VL CDR2, and VL CDR3, respectively, comprise the amino sequences of SEQ ID NO: 123-128. In some embodiments, the VH comprises an amino acid sequence selected from the group consisting of SEQ ID NO: 121 and 129-131, and the VL comprises an amino acid sequence selected from the group consisting of SEQ ID NO: 122 and 132-137. In some embodiments, the VH comprises the amino acid of SEQ ID NO: 130, and the VL comprises the amino acid sequence of SEQ ID NO: 133.

Also provided are compositions, methods, and uses for treating diseases. In one embodiment, it is for treating cancer in a patient in need thereof. In some embodiments, the treatment further comprises administration of an immune checkpoint inhibitor.

### BRIEF DESCRIPTION OF THE DRAWINGS

FIG. 1 shows the binding activity of the anti-CD40 monospecific antibodies to the human CD40 antigen, cyno CD40 antigen.

FIG. 2 shows the binding activity of the anti-CD40 monospecific antibodies to Jurkat cell overexpressing human CD40.

FIG. 3 presents ELISA binding results of anti-CD40 antibodies to human OX40 or human 4-1BB.

FIG. 4 shows the format of Claudin 18.2/CD40 bispecific antibodies.

FIG. 5 shows that the anti-Claudin 18.2/CD40 bispecific antibodies bound to human dendritic cells with a weaker activity than the benchmark antibody selicrelumab.

FIG. 6 shows that anti-Claudin 18.2/CD40 bispecific antibodies bound to human B cells with a weaker activity than the benchmark antibody selicrelumab.

FIG. 7 shows that CD40 monospecific antibodies active CD40 signaling with a much lower activity than the benchmark antibody selicrelumab.

FIG. 8 shows that anti-Claudin 18.2/CD40 bispecific antibodies activate CD40 signaling in a Claudin 18.2-dependent manner.

FIG. 9 shows that anti-Claudin 18.2/CD40 bispecific antibodies activate dendritic cells to secrete IL-12 in a Claudin 18.2-dependent manner.

FIG. 10 shows that anti-Claudin 18.2/CD40 bispecific antibodies activate dendritic cells to express CD80 (A) and CD86 (B) in a Claudin 18.2-dependent manner.

FIG. 11 shows that anti-Claudin 18.2/CD40 bispecific antibodies increase B cell proliferation (A) and activation (B) in a Claudin 18.2-dependent manner.

FIG. 12 shows the binding activity of humanized 2p442 antibodies to Jurkat cell overexpressing human CD40.

FIG. 13 shows that humanized anti-Claudin 18.2/CD40 bispecific antibodies had comparable CD40 activation activities to their chimeric antibodies.

FIG. 14 shows that humanized anti-Claudin 18.2/CD40 bispecific antibodies had comparable function in inducing IL-12 secretion to their chimeric antibodies.

FIG. 15 shows that humanized anti-Claudin 18.2/CD40 bispecific antibodies had comparable function in inducing CD80 (A) and CD86 (B) expression on dendritic cell to their chimeric antibodies.

FIG. 16 shows that humanized anti-Claudin 18.2/CD40 bispecific antibodies had comparable function in inducing B cell proliferation (A) and activation (B) to their chimeric antibodies.

FIG. 17 shows the in vivo tumor growth inhibition of anti-Claudin 18.2/CD40 chimeric bispecific antibodies.

FIG. 18 shows the in vivo study design (A) and blood biochemistry analysis (B), tumor infiltrating immunophenotyping (IPT) analysis (C-F) and spleen IPT analysis (G-I) of anti-Claudin 18.2/CD40 chimeric bispecific antibodies.

FIG. 19 shows that anti-5T4/CD40 bispecific antibodies with “2+2 b1l” format activate CD40 signaling in a 5T4-dependent manner.

FIG. 20 shows that anti-5T4/CD40 bispecific antibodies activate dendritic cells to secrete IL-12 in a 5T4-dependent manner.

FIG. 21 shows that anti-5T4/CD40 bispecific antibodies activate dendritic cells to express CD80 (A) and CD86 (B) in a 5T4-dependent manner.

FIG. 22 shows the “2+2 b11” format, “1+1 b12” format, “2+2 b13” format, “1+2 b16” format, “1+2 b17” format and “1+3 b18” format of anti-5T4/CD40 bispecific antibodies.

FIG. 23 shows the binding activity of the anti-5T4/CD40 bispecific antibodies to Jurkat cell overexpressing human CD40.

FIG. 24 shows that anti-5T4/CD40 bispecific antibodies with different formats activate CD40 signaling in different potency.

FIG. 25 shows that anti-5T4/CD40 bispecific antibodies with different formats activate dendritic cells to secrete IL-12 in different potency.

FIG. 26 shows the in vivo tumor growth inhibition of anti-5T4/CD40 bispecific antibodies.

FIG. 27 shows the ex vivo blood immunophenotyping (IPT) analysis of tested anti-5T4/CD40 bispecific antibodies.

FIG. 28 shows the ex vivo tumor infiltrating IPT analysis of tested anti-5T4/CD40 bispecific antibodies.

FIG. 29 shows that humanized anti-5T4/CD40 bispecific antibody b16 (42p155z2)-LALA had comparable binding activity to human dendritic cells as its chimeric antibody.

FIG. 30 shows that humanized anti-5T4/CD40 bispecific antibody b16 (42p155z2)-LALA had comparable CD40 activation activity as its chimeric antibody.

FIG. 31 shows that humanized anti-5T4/CD40 bispecific antibody of b16 (42p155z2)-LALA had comparable function in inducing IL-12 secretion as its chimeric antibody of.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters; Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Biologically equivalent polynucleotides are those having the above-noted specified percent homology and encoding a polypeptide having the same or similar biological activity.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

A single-domain antibody (sdAb), also known as a nanobody, is an antibody fragment consisting of a single monomeric variable antibody domain. Nanobodies produced from camelids and certain other animals are also referred to as VHH fragments. Like a whole antibody, a nanobody is able to bind selectively to a specific antigen. With a molecular weight of only 12-15 kDa, single domain antibodies are much smaller than common antibodies (150-160 kDa). Single domain antibodies, given their small sizes and one-chain nature, can be particularly suitable for inclusion as a fragment in other proteins, such chimeric antigen receptors (CAR) and bispecific antibodies.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Tumor Antigen-Dependent CD40 Agonist Antibodies for Improved Therapeutic Index

CD40 is expressed broadly in hematopoietic and nonhematopoietic tissues. CD40 regulates immunity and thus provides a potential pathway for cancer immunotherapy. It has been shown that activation of CD40 can license DCs to drive CD8 T cell responses. Moreover, CD40 activation accomplishes immune activation independently of innate immune receptors such as stimulator of interferon genes (STING) or Toll-like receptors (TLRs). Accordingly, there have been serious efforts to develop CD40 agonist antibodies for treating cancer.

The agonist approach, however, poses major challenges around dose and schedule that complicate drug development, unlike inhibiting antibodies. A further complicating factor is that CD40 agonist antibodies have been commonly associated with moderate to severe toxicities such as cytokine release syndrome (CRS). This is likely due to CD40 activation at non-cancerous sites. The current solution is to treat these toxicities when they occur, which is difficult to manage, is expensive, and deters patients.

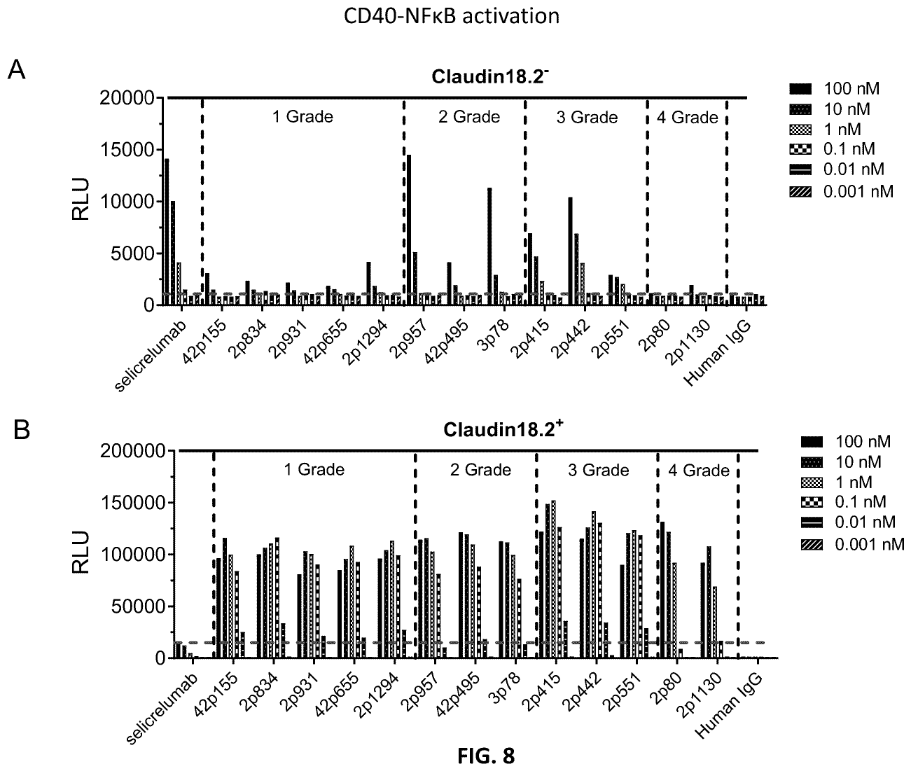

Through careful design and screening, unexpectedly, the instant inventors were able to identify CD40 agonist antibodies having varying CD40 activation activities in the presence or absence of tumor-associated antigens (TAA). In particular, a long listing of new CD40 antibodies were identified all of which had considerably reduced CD40 agonist activities as compared to the reference antibody selicrelumab (see, e.g., Example 4 and FIG. 8A), the most extensively studied CD40 antibody candidate. However, when used in a bi- or multi-specific format that further included an anti-TAA unit, these antibodies exhibited much greater CD40 activation activity than selicrelumab (see, e.g., FIG. 8B, 9-11).

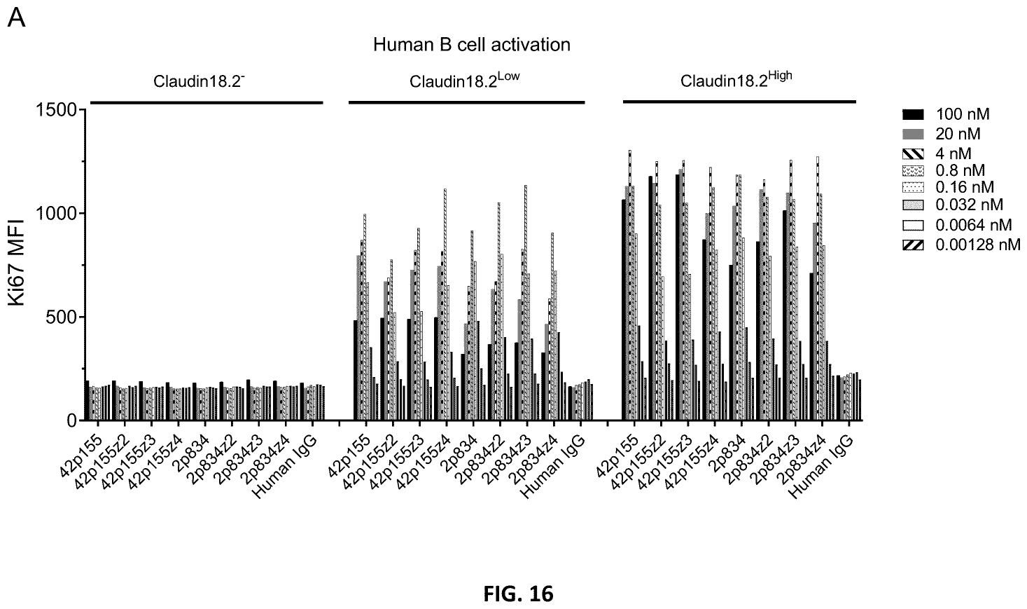

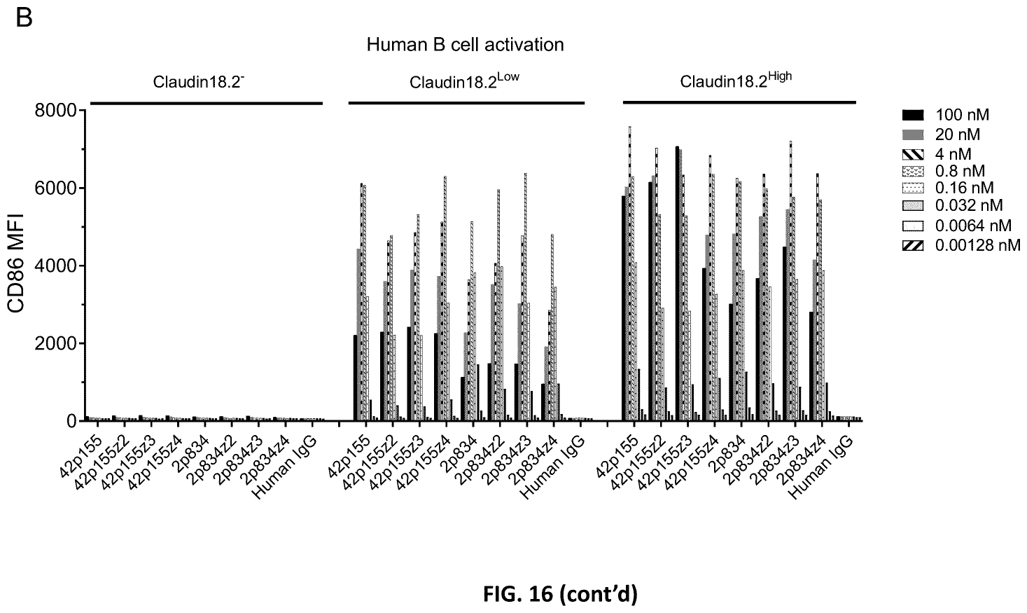

These antibodies, therefore, can be referred to as “TAA-dependent CD40 agonist antibodies.” These TAA-dependent CD40 agonist antibodies showed greatly improved anti-tumor efficacy in animal models (see, e.g., Example 9 and FIG. 16). It also necessarily follows that they would cause greatly reduced toxicities as they do not induce CD40 activities in tissue or organs that do not express the targeted TAA.

More interestingly, these newly identified CD40 agonist antibodies were further categorized into four grades. As shown in FIG. 8 and summarized in Table 5, Grade 4 antibodies did not activate CD40 in the absence of TAA and only had relatively weak CD40 activation in the presence of TAA; Grade 3 had moderate CD40 activation in the absence of TAA and the most potent CD40 activation in the presence of TAA; and Grade 2 had marginal CD40 activation in the absence of TAA and moderate CD40 activation in the presence of TAA.

Grade 1 antibodies are the most interesting. They had low level or no CD40 activation in the absence of TAA and potent CD40 activation when the TAA was present on the target cells. Grade 1 antibodies include 42p155, 2p834, 2p931, 42p655, and 2p1294. These antibodies are believed to have the highest therapeutic index and thus greatest clinical potential.

Accordingly, in one embodiment of the present disclosure, provided are TAA-dependent CD40 agonist antibodies. A TAA-dependent CD40 agonist antibody is one that, when presented in a bi- or multi-specific antibody format that further includes an anti-TAA unit, activates CD40 only on a TAA-expressing cell, and more than CD40 on a reference TAA-absent cell. For fair comparison, in some embodiments, the reference cell differs from the TAA-expressing cell only differs by the expression of the TAA.

In some embodiments, the difference for CD40 activation between TAA-expressing and TAA-absent cells is at least 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold, 30 fold, 35 fold, 40 fold, 45 fold, 50 fold, or 100 fold.

In some embodiments, the TAA-dependent CD40 agonist antibody activates CD40 less than selicrelumab, such as 10%, 20%, 30%, 40%, 50%, 60%, 70%, 80% or 90% less than selicrelumab, in the absence of the TAA.

In some embodiments, the TAA on a TAA-expressing cell is at least detectable by routine means, such as immunohistochemical staining. In some embodiments, the TAA expression is at least at a level that is average to tumors that express the TAA.

In some embodiments, the activation measurement is conducted with the bi- or multi-specific antibody is present at a concentration that is from 0.001 nM to 1000 nM. In some embodiments, the antibody concentration is from 0.01 nM to 500 nM. In some embodiments, the antibody concentration is from 0.1 nM to 200 nM. In some embodiments, the antibody concentration is from 0.1 nM to 20 nM. In some embodiments, the antibody concentration is from 1 nM to 20 nM. In some embodiments, the antibody concentration is about 0.001, 0.01, 0.1, 0.14, 1, 1.2, 11 or 100 nM.

Different assays for measuring CD40 activation are available, including with commercially available kits. In one example, the target cell is a CHO cell and the activation is measured with a NFκB reporter assay. In another example, the target cell is a dendric cell (DC) and the CD40 activation is measured with IL-12 secretion, CD80 and CD86 expression. In yet another example, the target cell is a B cell and the CD40 activation is measured with Ki67 and/or CD86 expression.

Example TAA-dependent CD40 agonist antibodies are provided as well, long with proteins (e.g., multi-specific antibodies, chimeric antigen receptors (CAR)). In one embodiment of the present disclosure, provided are single domain antibodies and polypeptides that include such a single domain antibody. In one embodiment of the present disclosure, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 42p155 (SEQ ID NO:1). In some embodiments, the CDR1, CDR2 and CDR3 include the amino acid sequences of SEQ ID NO: 14-16, respectively.

Certain residues in the CDR2 of 42p155, analysis shows, can potentially be subject to post-translational modifications (PTM). Accordingly, mutations were made to prevent such PTM (hence referred to as PTM derisked versions), including NG=>NA or QG. See, e.g., SEQ ID NO:63 and 64. In some embodiments, therefore, the CDR1, CDR2 and CDR3 include the amino acid sequences of SEQ ID NO:14, 63, and 16, respectively. In some embodiments, therefore, the CDR1, CDR2 and CDR3 include the amino acid sequences of SEQ ID NO:14, 64, and 16, respectively.

In some embodiments, humanized versions of 42p155 and its PTM derisked counterparts are also provided, such as those provided in SEQ ID NO:53-62. In some embodiments, the humanized antibodies include back mutations selected from the group consisting of IP, 2S, 88P, and 98Q, according to Kabat numbering. In some embodiments, the humanized antibodies include back mutation 98Q. In some embodiments, the humanized antibodies include back mutations 88P and 98Q. In some embodiments, the humanized antibodies include back mutations IP, 2S, 88P, and 98Q.

In some embodiments, in the humanized version, the CDR1 includes the amino acid sequence of SEQ ID NO:14, the CDR2 includes the amino acid sequence of SEQ ID NO:15, and the CDR3 includes the amino acid sequence of SEQ ID NO:16. In some embodiments, the antibody or polypeptide includes an amino acid sequence selected from the group consisting of SEQ ID NO: 53, 54, 57 and 60. In some embodiments, the antibody includes the recited CDR1, CDR2 and CDR3 and has at least 80%, 85%, 90%, 95%, 98%, or 99% sequence identity to SEQ ID NO: 53, 54, 57 or 60.

In some embodiments, in the humanized version, the CDR 1 includes the amino acid sequence of SEQ ID NO: 14, the CDR2 includes the amino acid sequence of SEQ ID NO:63, and the CDR3 includes the amino acid sequence of SEQ ID NO: 16. In some embodiments, the antibody or polypeptide includes an amino acid sequence selected from the group consisting of SEQ ID NO: 55, 58 and 61. In some embodiments, the antibody includes the recited CDR1, CDR2 and CDR3 and has at least 80%, 85%, 90%, 95%, 98%, or 99% sequence identity to SEQ ID NO: 55, 58 or 61.

In some embodiments, in the humanized version, the CDR1 includes the amino acid sequence of SEQ ID NO: 14, the CDR2 includes the amino acid sequence of SEQ ID NO:64, and the CDR3 includes the amino acid sequence of SEQ ID NO:16. In some embodiments, the antibody or polypeptide includes an amino acid sequence selected from the group consisting of SEQ ID NO: 56, 59 and 62. In some embodiments, the antibody includes the recited CDR1, CDR2 and CDR3 and has at least 80%, 85%, 90%, 95%, 98%, or 99% sequence identity to SEQ ID NO: 56, 59 or 62.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 2p834 (SEQ ID NO:2). In some embodiments, the CDR1, CDR2 and CDR3 include the amino acid sequences of SEQ ID NO:17-19, respectively.

In some embodiments, humanized versions of 2p834 are also provided, such as those provided in SEQ ID NO:65-68. In some embodiments, the humanized antibodies include back mutations selected from the group consisting of IP, 2S, 88P, and 98Q, according to Kabat numbering. In some embodiments, the humanized antibodies include back mutation 98Q. In some embodiments, the humanized antibodies include back mutations 88P and 98Q. In some embodiments, the humanized antibodies include back mutations IP, 2S, 88P, and 98Q. In some embodiments, the antibody includes the recited CDR1, CDR2 and CDR3 and has at least 80%, 85%, 90%, 95%, 98%, or 99% sequence identity to SEQ ID NO: 1, 65, 66, 67, or 68.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 2p931 (SEQ ID NO:3). In some embodiments, the CDR1, CDR2 and CDR3 include the amino acid sequences of SEQ ID NO:20-22, respectively. In some embodiments, the antibody includes the recited CDR1, CDR2 and CDR3 and has at least 80%, 85%, 90%, 95%, 98%, or 99% sequence identity to SEQ ID NO:3.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 42p655 (SEQ ID NO:4). In some embodiments, the CDR1, CDR2 and CDR3 include the amino acid sequences of SEQ ID NO:23-25, respectively. In some embodiments, the antibody includes the recited CDR1, CDR2 and CDR3 and has at least 80%, 85%, 90%, 95%, 98%, or 99% sequence identity to SEQ ID NO:4.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 2p1294 (SEQ ID NO:5). In some embodiments, the CDR1, CDR2 and CDR3 include the amino acid sequences of SEQ ID NO:26-28, respectively. In some embodiments, the antibody includes the recited CDR1, CDR2 and CDR3 and has at least 80%, 85%, 90%, 95%, 98%, or 99% sequence identity to SEQ ID NO:5.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 2p957 (SEQ ID NO:6). In some embodiments, the CDR1. CDR2 and CDR3 include the amino acid sequences of SEQ ID NO:29-31, respectively. In some embodiments, the antibody includes the recited CDR1, CDR2 and CDR3 and has at least 80%, 85%, 90%, 95%, 98%, or 99% sequence identity to SEQ ID NO:6.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 42p495 (SEQ ID NO:7). In some embodiments, the CDR1, CDR2 and CDR3 include the amino acid sequences of SEQ ID NO:32-34, respectively. In some embodiments, the antibody includes the recited CDR1, CDR2 and CDR3 and has at least 80%, 85%, 90%, 95%, 98%, or 99% sequence identity to SEQ ID NO:7.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 3p78 (SEQ ID NO:8). In some embodiments, the CDR1, CDR2 and CDR3 include the amino acid sequences of SEQ ID NO:35-37, respectively. In some embodiments, the antibody includes the recited CDR1, CDR2 and CDR3 and has at least 80%, 85%, 90%, 95%, 98%, or 99% sequence identity to SEQ ID NO:8.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 2p415 (SEQ ID NO:9). In some embodiments, the CDR1, CDR2 and CDR3 include the amino acid sequences of SEQ ID NO:38-40, respectively. In some embodiments, the antibody includes the recited CDR1, CDR2 and CDR3 and has at least 80%, 85%, 90%, 95%, 98%, or 99% sequence identity to SEQ ID NO:9.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 2p442 (SEQ ID NO: 10). In some embodiments, the CDR1, CDR2 and CDR3 include the amino acid sequences of SEQ ID NO:41-43, respectively. In some embodiments. the antibody includes the recited CDR1, CDR2 and CDR3 and has at least 80%, 85%, 90%, 95%, 98%, or 99% sequence identity to SEQ ID NO:10.

In some embodiments, humanized versions of 2p442 are also provided, such as those provided in SEQ ID NO:69-72. In some embodiments, the antibody includes the recited CDR1. CDR2 and CDR3 and has at least 80%, 85%, 90%, 95%, 98%, or 99% sequence identity to SEQ ID NO: 10, 69, 70, 71 or 72.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 2p551 (SEQ ID NO:11). In some embodiments, the CDR1, CDR2 and CDR3 include the amino acid sequences of SEQ ID NO:44-46, respectively. In some embodiments. the antibody includes the recited CDR1, CDR2 and CDR3 and has at least 80%, 85%, 90%, 95%, 98%, or 99% sequence identity to SEQ ID NO:11.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 2p80 (SEQ ID NO: 12). In some embodiments, the CDR1, CDR2 and CDR3 include the amino acid sequences of SEQ ID NO:47-49, respectively. In some embodiments, the antibody includes the recited CDR1, CDR2 and CDR3 and has at least 80%, 85%, 90%, 95%, 98%, or 99% sequence identity to SEQ ID NO: 12.

In another embodiment, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 2p1130 (SEQ ID NO:13). In some embodiments, the CDR1, CDR2 and CDR3 include the amino acid sequences of SEQ ID NO:50-52, respectively. In some embodiments, the antibody includes the recited CDR1, CDR2 and CDR3 and has at least 80%, 85%, 90%, 95%, 98%, or 99% sequence identity to SEQ ID NO: 14.

Also provided, in some embodiments, are anti-CD40 antibodies and antigen binding fragments that compete with any of the antibodies disclosed herein in binding to human CD40. Also provided, in some embodiments, are anti-CD40 antibodies and antigen binding fragments that bind to the same epitope as any of the antibodies disclosed herein. Also provided, in some embodiments, are anti-CD40 antibodies and antigen binding fragments that included the CDR1, CDR2, and CDR3 of the antibodies disclosed herein.

Also provided are compositions that include the antibody or the polypeptide and a pharmaceutically acceptable carrier.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence. In some embodiments, the modified antibody or fragment retains the designate CDR sequences.

In certain embodiments, the antibody includes an amino acid sequence or one or more moieties not normally associated with an antibody. Exemplary modifications are described in more detail below. For example, an antibody of the disclosure may include a flexible linker sequence, or may be modified to add a functional moiety (e.g., PEG, a drug, a toxin, or a label).

### Bispecific and Multispecific Antibodies

As provided, the CD40 agonist antibodies disclosed here are particularly useful for preparing bispecific and multispecific antibodies. This is at least because of these antibodies' enhanced therapeutic index and their small sizes.

CD40 is expressed broadly in hematopoietic and nonhematopoietic tissues. CD40 regulates immunity and thus provides a potential pathway for cancer immunotherapy. It has been shown that activation of CD40 can license DCs to drive CD8 T cell responses. Moreover, CD40 activation accomplishes immune activation independently of innate immune receptors such as stimulator of interferon genes (STING) or Toll-like receptors (TLRs). Accordingly, there have been serious efforts to develop CD40 agonist antibodies for treating cancer.

The agonist approach, however, poses major challenges around dose and schedule that complicate drug development, unlike inhibiting antibodies. A further complicating factor is that CD40 agonist antibodies have been commonly associated with moderate to severe toxicities such as cytokine release syndrome (CRS). This is likely due to CD40 activation at non-cancerous sites. The current solution is to treat these toxicities when they occur, which is difficult to manage, is expensive, and deters patients.

Accordingly, in one embodiment, provided is a bispecific antibody that includes a TAA-dependent CD40 agonist antibody, or an antigen-binding fragment thereof, and a second antibody or antigen-binding fragment having binding specificity to a target antigen that is not CD40. In some embodiment, a third or fourth specificity is further included. The target antigen that is not CD40, in some embodiments, is a tumor antigen.

A TAA-dependent CD40 agonist antibody is one that, when presented in such a bi- or multi-specific antibody format that further includes an anti-TAA unit, activates CD40 on a TAA-expressing cell more than CD40 on a reference TAA-absent cell. For fair comparison, in some embodiments, the reference cell differs from the TAA-expressing cell only differs by the expression of the TAA.

In some embodiments, the difference for CD40 activation between TAA-expressing and TAA-absent cells is at least 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold, 30 fold, 35 fold, 40 fold, 45 fold, or 50 fold.

In some embodiments, the TAA-dependent CD40 agonist antibody activates CD40 less than selicrelumab, such as 10%, 20%, 30%, 40%, 50%, 60%, 70%, 80% or 90% less than selicrelumab, in the absence of the TAA.

In some embodiments, the TAA on a TAA-expressing cell is at least detectable by routine means, such as immunohistochemical staining. In some embodiments, the TAA expression is at least at a level that is average to tumors that express the TAA.

In some embodiments, the activation measurement is conducted with the bi- or multi-specific antibody is present at a concentration that is from 0.001 nM to 1000 nM. In some embodiments, the antibody concentration is from 0.01 nM to 500 nM. In some embodiments, the antibody concentration is from 0.1 nM to 200 nM. In some embodiments, the antibody concentration is from 0.1 nM to 20 nM. In some embodiments, the antibody concentration is from 1 nM to 20 nM. In some embodiments, the antibody concentration is about 0.001, 0.01, 0.1, 0.14, 1, 1.2, 11 or 100 nM.

Different assays for measuring CD40 activation are available, including with commercially available kits. In one example, the target cell is a CHO cell and the activation is measured with a NFκB reporter assay. In another example, the target cell is a dendric cell (DC) and the CD40 activation is measured with IL-12 secretion, CD80 or CD86 expression. In yet another example, the target cell is a B cell and the CD40 activation is measured with Ki67 and/or CD86 expression.

In some embodiments, the TAA-dependent CD40 agonist antibody is as disclosed in the preceding section, such as 42p155, 2p834, 2p931, 42p655, 2p1294, 2p957, 42p495, 3p78, 2p415, 2p442, 2p551, 2p80 and 2p1 130 and their biological equivalents.

An abundance of tumor antigens are known in the art and new tumor antigens can be readily identified by screening. Non-limiting examples of tumor antigens include Claudin 18.2, EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGFIR, EPHA3, TRAILR1, TRAILR2, RANKL, FAP and Tenascin. In some embodiments, the bispecific antibody has specificity to CD40 and Claudin 18.2.

The instant inventors have designed a panel of 5T4×CD40 bispecific antibodies, with anti-5T4/anti-CD40 portions of different characteristics, and in different formats. These bispecific antibodies were evaluated in CD40 reporter cells and cocultured with target cells expressing 5T4. The potency was further confirmed in vitro by measuring IL12 production from monocyte-derived dendritic cells (DCs) and CD80 and CD86 expression on the DC and B cells. Further, in vivo anti-tumor efficacy was determined in CD40-humanized C57BL/6 mice bearing MC38-hu5T4 tumor.

It has been observed that bispecific antibodies that activate CD40 signaling in a 5T4-dependent manner exhibited the best in vitro and in vivo performance. Moreover, multiple formats of bispecific antibodies were tested, and two of them (b16 and b18) showed superiority in inducing more potent CD40 agonism, in a 5T4-dependent manner.

In vivo testing with 5T4×CD40 demonstrated potent anti-tumor efficacy, significantly higher than the clinical benchmark at similar doses. Furthermore, the resulting tumor-free mice were resistant to tumor rechallenge, demonstrating an established long-lasting memory response. Moreover, ex vivo analysis showed a focused immune activation in the tumor and no peripheral activation, ensuring safety of these bispecific antibodies.

A. Bispecific Antibodies with 5T4-Dependent Anti-CD40 Portion

In accordance with one embodiment of the present disclosure, provided is a bispecific antibody, or a multi-specific antibody that incorporates the bispecific antibody, that includes an anti-5T4 portion and an anti-CD40 portion. In some embodiments, the anti-CD40 portion includes one, two, three, or four anti-CD40 antibodies or fragments having a 5T4-dependent agonist activity.

The instant inventors have prepared and tested single domain anti-CD40 antibodies have considerably reduced CD40 agonist activities as compared to the reference antibody selicrelumab, the most extensively studied CD40 antibody candidate. When used in a bi- or multi-specific format that further included an anti-tumor-associated antigen (TAA, e.g., 5T4) unit, these antibodies exhibited much greater CD40 activation activity than selicrelumab. These antibodies, therefore, can be referred to as “5T4-dependent CD40 agonist antibodies.” These 5T4-dependent CD40 agonist antibodies showed greatly improved anti-tumor efficacy in animal models. It also necessarily follows that they would cause greatly reduced toxicities as they do not induce CD40 activities in tissue or organs that do not express 5T4.

A 5T4-dependent CD40 agonist antibody is one that, when presented in a bi- or multi-specific antibody format that further includes an anti-5T4unit, activates CD40 only on a 5T4-expressing cell, and more than CD40 on a reference 5T4-absent cell. For fair comparison, in some embodiments, the reference cell differs from the 5T4-expressing cell only differs by the expression of the TAA.

In some embodiments, the difference for CD40 activation between 5T4-expressing and 5T4-absent cells is at least 2 fold, 3 fold, 4 fold, 5 fold, 6 fold, 7 fold, 8 fold, 9 fold, 10 fold, 11 fold, 12 fold, 13 fold, 14 fold, 15 fold, 16 fold, 17 fold, 18 fold, 19 fold, 20 fold, 21 fold, 22 fold, 23 fold, 24 fold, 25 fold, 26 fold, 27 fold, 28 fold, 29 fold, 30 fold, 35 fold, 40 fold, 45 fold, 50 fold, or 100 fold.

In some embodiments, the 5T4-dependent CD40 agonist antibody activates CD40 less than selicrelumab, such as 10%, 20%, 30%, 40%, 50%, 60%, 70%, 80% or 90% less than selicrelumab, in the absence of the 5T4.

In some embodiments, the 5T4 on a 5T4-expressing cell is at least detectable by routine means, such as immunohistochemical staining. In some embodiments, the 5T4 expression is at least at a level that is average to tumors that express the 5T4.

In some embodiments, the activation measurement is conducted with the bi- or multi-specific antibody is present at a concentration that is from 0.001 nM to 1000 nM. In some embodiments, the antibody concentration is from 0.01 nM to 500 nM. In some embodiments, the antibody concentration is from 0.1 nM to 200 nM. In some embodiments, the antibody concentration is from 0.1 nM to 20 nM. In some embodiments, the antibody concentration is from 1 nM to 20 nM. In some embodiments, the antibody concentration is about 0.001, 0.01, 0.1, 0.14, 1, 1.2, 11 or 100 nM.

Different assays for measuring CD40 activation are available, including with commercially available kits. In one example, the target cell is a CHO cell and the activation is measured with a NFκB reporter assay. In another example, the target cell is a dendric cell (DC) and the CD40 activation is measured with IL-12 secretion, CD80 and CD86 expression. In yet another example, the target cell is a B cell and the CD40 activation is measured with Ki67 and/or CD86 expression.

Example 5T4-dependent CD40 agonist antibodies are provided as well, long with proteins (e.g., multi-specific antibodies, chimeric antigen receptors (CAR)). In one embodiment of the present disclosure, provided are single domain antibodies and polypeptides that include such a single domain antibody. In one embodiment of the present disclosure, provided is a single domain antibody or a polypeptide that includes the single domain antibody, wherein the single domain antibody includes a CDR1, a CDR2 and a CDR3, which respectively have the CDR1, CDR2 and CDR3 sequences of antibody 42p155 (SEQ ID NO: 1). In some embodiments, the CDR1, CDR2 and CDR3 include the amino acid sequences of SEQ ID NO: 14-16, respectively.

Certain residues in the CDR2 of 42p155, analysis shows, can potentially be subject to post-translational modifications (PTM). Accordingly, mutations were made to prevent such PTM (hence referred to as PTM derisked versions), including NG=>NA or QG. See, e.g., SEQ ID NO: 63 and 64. In some embodiments, therefore, the CDR1, CDR2 and CDR3 include the amino acid sequences of SEQ ID NO: 14, 63, and 16, respectively. In some embodiments, therefore, the CDR1, CDR2 and CDR3 include the amino acid sequences of SEQ ID NO: 14, 64, and 16, respectively.

In some embodiments, humanized versions of 42p155 and its PTM derisked counterparts are also provided, such as those provided in SEQ ID NO: 53-62. In some embodiments, the humanized antibodies include back mutations selected from the group consisting of IP, 2S, 88P, and 98Q, according to Kabat numbering. In some embodiments, the humanized antibodies include back mutation 98Q. In some embodiments, the humanized antibodies include back mutations 88P and 98Q. In some embodiments, the humanized antibodies include back mutations IP, 2S, 88P, and 98Q.

In some embodiments, in the humanized version, the CDR1 includes the amino acid sequence of SEQ ID NO: 14, the CDR2 includes the amino acid sequence of SEQ ID NO:15, and the CDR3 includes the amino acid sequence of SEQ ID NO: 16. In some embodiments, the antibody or polypeptide includes an amino acid sequence selected from the group consisting of SEQ ID NO: 53, 54, 57 and 60. In some embodiments, the antibody includes the recited CDR1, CDR2 and CDR3 and has at least 80%, 85%, 90%, 95%, 98%, or 99% sequence identity to any one of SEQ ID NO: 53, 54, 57 and 60.

In some embodiments, in the humanized version, the CDR1 includes the amino acid sequence of SEQ ID NO: 14, the CDR2 includes the amino acid sequence of SEQ ID NO: 63, and the CDR3 includes the amino acid sequence of SEQ ID NO: 16. In some embodiments, the antibody or polypeptide includes an amino acid sequence selected from the group consisting of SEQ ID NO: 55, 58 and 61. In some embodiments, the antibody includes the recited CDR1, CDR2 and CDR3 and has at least 80%, 85%, 90%, 95%, 98%, or 99% sequence identity to any one of SEQ ID NO: 55, 58 and 61.

In some embodiments, in the humanized version, the CDR1 includes the amino acid sequence of SEQ ID NO: 14, the CDR2 includes the amino acid sequence of SEQ ID NO: 64, and the CDR3 includes the amino acid sequence of SEQ ID NO: 16. In some embodiments, the antibody or polypeptide includes an amino acid sequence selected from the group consisting of SEQ ID NO: 56, 59 and 62. In some embodiments, the antibody includes the recited CDR1, CDR2 and CDR3 and has at least 80%, 85%, 90%, 95%, 98%, or 99% sequence identity to any one of SEQ ID NO: 56, 59 and 62.

Also provided, in some embodiments, are anti-CD40) antibodies and antigen binding fragments that compete with any of the antibodies disclosed herein in binding to human CD40. Also provided, in some embodiments, are anti-CD40) antibodies and antigen binding fragments that bind to the same epitope as any of the antibodies disclosed herein. Also provided, in some embodiments, are anti-CD40) antibodies and antigen binding fragments that included the CDR1, CDR2, and CDR3 of the antibodies disclosed herein.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence. In some embodiments, the modified antibody or fragment retains the designate CDR sequences.

B. Bispecific Antibodies with Anti-5T4 Portion Targeting Bin A or B

In accordance with one embodiment of the present disclosure, provided is a bispecific antibody, or a multi-specific antibody that incorporates the bispecific antibody, that includes an anti-5T4 portion and an anti-CD40 portion. In some embodiments, the anti-5T4 portion includes one or more anti-5T4 antibodies or fragments that competes with bin A or bin B antibodies in binding to the human 5T4 protein.

As provided in Example 10, all anti-5T4 antibodies disclosed herein can be categorized, according to binding competition assays, into four bins, A-D. Bin A includes the antibody from naptumomab, along with new antibodies 14G12 and 393E9; bin B includes 159D5, and bin D includes 286B4. As reported in Example 12, antibodies of bins B and C exhibited superior agonist activities.

Anti-5T4 antibodies and antigen-binding fragments of bin A can be represented by antibody 14G12, its humanized and de-risked versions, and those that compete with 14G12 in binding to the human 5T4 protein.

In one embodiment of the present disclosure, the anti-5T4 protein includes an antibody or antigen-binding fragment, which includes a heavy chain variable region (VH) that includes a CDR1, a CDR2 and a CDR3, and a light chain variable region (VL) that includes a CDR1, a CDR2 and a CDR3. In some embodiments, the VH CDR1, VH CDR2, VH CDR3, VL CDR1, VL CDR2 and VL CDR3 include the sequences of SEQ ID NO: 75-80, respectively.

In some embodiments, the VH and VL include the sequence of SEQ ID NO: 73 and 74, respectively. In some embodiments, the VH includes the sequence of any one of SEQ ID NO: 81-90 and the VL includes the sequence of any one of SEQ ID NO: 91-100. In some embodiments, the VH includes the recited VH CDR1, CDR2 and CDR3 and has at least 80%, 85%, 90%, 95%, 98%, or 99% sequence identity to any one of SEQ ID NO: 73 and 81-90, and the VL includes the recited VL CDR1, CDR2 and CDR3 and has at least 80%, 85%, 90%, 95%, 98%, or 99% sequence identity to any one of SEQ ID NO: 74 and 91-100.

In some embodiments, the VH and VL include the sequence of SEQ ID NO: 83 and 91, respectively. In some embodiments, the VH includes the recited VH CDR1, CDR2 and CDR3 and has at least 80%, 85%, 90%, 95%, 98%, or 99% sequence identity to SEQ ID NO: 83, and the VL includes the recited VL CDR1, CDR2 and CDR3 and has at least 80%, 85%, 90%, 95%, 98%, or 99% sequence identity to SEQ ID NO: 91.

In some embodiments, the VH and VL include the sequence of SEQ ID NO: 89 and 91, respectively. In some embodiments, the VH includes the recited VH CDR1, CDR2 and CDR3 and has at least 80%, 85%, 90%, 95%, 98%, or 99% sequence identity to SEQ ID NO: 89, and the VL includes the recited VL CDR1, CDR2 and CDR3 and has at least 80%, 85%, 90%, 95%, 98%, or 99% sequence identity to SEQ ID NO: 91.

Anti-5T4 antibodies and antigen-binding fragments of bin A can also be represented by antibody 393E9, its humanized and de-risked versions, and those that compete with 393E9 in binding to the human 5T4 protein.

In one embodiment of the present disclosure, the anti-5T4 protein includes an antibody or antigen-binding fragment, which includes a heavy chain variable region (VH) that includes a CDR1, a CDR2 and a CDR3, and a light chain variable region (VL) that includes a CDR1, a CDR2 and a CDR3. In some embodiments, the VH CDR1, VH CDR2. VH CDR3, VL CDR1, VL CDR2 and VL CDR3 include the sequences of SEQ ID NO: 103-108, respectively.

In some embodiments, the VH and VL include the sequence of SEQ ID NO: 101 and 102, respectively. In some embodiments, the VH includes the sequence of any one of SEQ ID NO: 109-115 and the VL includes the sequence of any one of SEQ ID NO: 116-120. In some embodiments, the VH includes the recited VH CDR1, CDR2 and CDR3 and has at least 80%, 85%, 90%, 95%, 98%, or 99% sequence identity to any one of SEQ ID NO: 101 and 109-115, and the VL includes the recited VL CDR1, CDR2 and CDR3 and has at least 80%, 85%, 90%, 95%, 98%, or 99% sequence identity to any one of SEQ ID NO: 102 and 116-120.

In some embodiments, the VH and VL include the sequence of SEQ ID NO: 113 and 120, respectively. In some embodiments, the VH includes the recited VH CDR1, CDR2 and CDR3 and has at least 80%, 85%, 90%, 95%, 98%, or 99% sequence identity to SEQ ID NO: 113, and the VL includes the recited VL CDR1, CDR2 and CDR3 and has at least 80%, 85%, 90%, 95%, 98%, or 99% sequence identity to SEQ ID NO: 120.

Anti-5T4 antibodies and antigen-binding fragments of bin B can be represented by antibody 159D5, its humanized and de-risked versions, and those that compete with 159D5 in binding to the human 5T4 protein.

In one embodiment of the present disclosure, the anti-5T4 protein includes an antibody or antigen-binding fragment, which includes a heavy chain variable region (VH) that includes a CDR1, a CDR2 and a CDR3, and a light chain variable region (VL) that includes a CDR1, a CDR2 and a CDR3. In some embodiments, the VH CDR1, VH CDR2. VH CDR3, VL CDR1, VL CDR2 and VL CDR3 include the sequences of SEQ ID NO: 123-128, respectively.

In some embodiments, the VH and VL include the sequence of SEQ ID NO: 121 and 122, respectively. In some embodiments, the VH includes the sequence of any one of SEQ ID NO: 129-131 and the VL includes the sequence of any one of SEQ ID NO: 132-137. In some embodiments, the VH includes the recited VH CDR1, CDR2 and CDR3 and has at least 80%, 85%, 90%, 95%, 98%, or 99% sequence identity to any one of SEQ ID NO: 121 and 129-131, and the VL includes the recited VL CDR1, CDR2 and CDR3 and has at least 80%, 85%, 90%, 95%, 98%, or 99% sequence identity to any one of SEQ ID NO: 122 and 132-137.

In some embodiments, the VH and VL include the sequence of SEQ ID NO: 130 and 133, respectively. In some embodiments, the VH includes the recited VH CDR1, CDR2 and CDR3 and has at least 80%, 85%, 90%, 95%, 98%, or 99% sequence identity to SEQ ID NO: 130, and the VL includes the recited VL CDR1, CDR2 and CDR3 and has at least 80%, 85%, 90%, 95%, 98%, or 99% sequence identity to SEQ ID NO: 133.

In certain embodiments, for certain desired activity, the anti-5T4 portion may include an antibody or antigen-binding fragments of bin D, which can be represented by antibody 286B4, its humanized and de-risked versions, and those that compete with 286B4 in binding to the human 5T4 protein.

In one embodiment of the present disclosure, the anti-5T4 protein includes an antibody or antigen-binding fragment, which includes a heavy chain variable region (VH) that includes a CDR1, a CDR2 and a CDR3, and a light chain variable region (VL) that includes a CDR1, a CDR2 and a CDR3. In some embodiments, the VH CDR1, VH CDR2, VH CDR3, VL CDR1, VL CDR2 and VL CDR3 include the sequences of SEQ ID NO: 140-145, respectively.

In some embodiments, the VH and VL include the sequence of SEQ ID NO: 138 and 139, respectively. In some embodiments, the VH includes the sequence of any one of SEQ ID NO: 146-151 and the VL includes the sequence of any one of SEQ ID NO: 152-157. In some embodiments, the VH includes the recited VH CDR1, CDR2 and CDR3 and has at least 80%, 85%, 90%, 95%, 98%, or 99% sequence identity to any one of SEQ ID NO: 138 and 146-151, and the VL includes the recited VL CDR1, CDR2 and CDR3 and has at least 80%, 85%, 90%, 95%, 98%, or 99% sequence identity to any one of SEQ ID NO: 139 and 152-157.

C. Bispecific Antibodies of Formats b11, b16 and b18

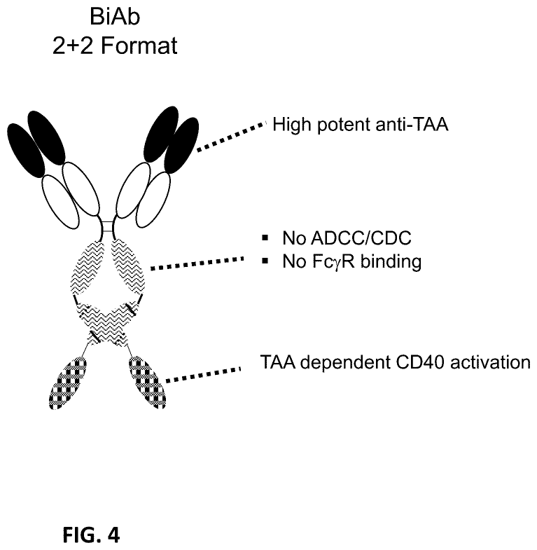

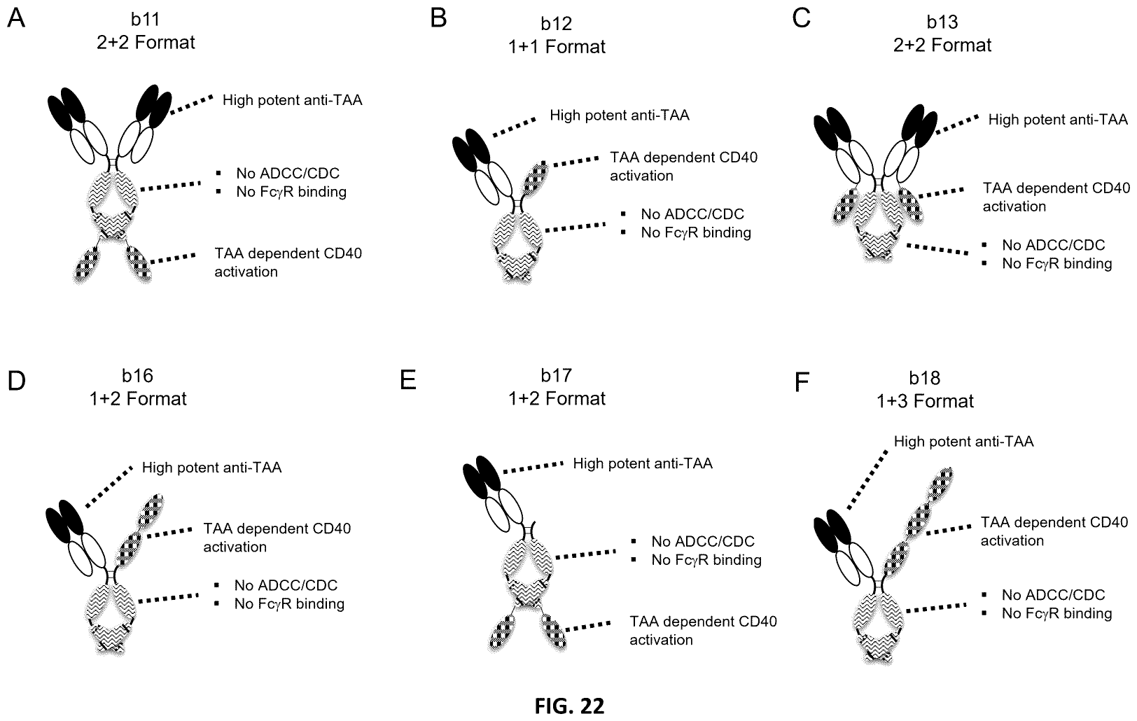

Multiple bispecific antibody formats have been tested in the accompanying examples. Format b11 (FIGS. 4 and 22A) exhibited excellent activity profile, while formats b16 (FIG. 22D) and b18 (FIG. 22F) showed even better performance.

In format b11, each of two anti-CD40) VHH antibodies is fused to the C-terminus of a conventional anti-5T4 antibody. This 2+2 format, therefore, is bivalent to both 5T4 and CD40. The Fc portion of the conventional anti-5T4 antibody can be optionally mutated to be deprived of ADCC/CDC activities or prevented from binding to FcγR. Example mutations include leucine (L) to alanine (A) substitution at the position 234 and 235 (LALA) and the alanine (A) to asparagine (N) substitution at position 297 (N297A).

In format b16, one of the VH/VL pairs in a conventional Fab 5T4 antibody is replaced with two concatenated anti-CD40 VHH antibodies. This 1+2 format, therefore, is a heterodimer. The Fc portion can be optionally mutated to be deprived of ADCC/CDC activities or prevented from binding to FcγR. Example mutations include leucine (L) to alanine (A) substitution at the position 234 and 235 (LALA) and the alanine (A) to asparagine (N) substitution at position 297 (N297A). In addition, the Fc fragment can be mutated to incorporate a knob-in-hole to reduce mispairing.

Format b18 differs from format b16 in that the anti-CD40 arm includes 3, not just 2. VHH anti-CD40) antibodies. Likewise, the Fc portion can be optionally mutated to be deprived of ADCC/CDC activities or prevented from binding to FcγR. Example mutations include leucine (L) to alanine (A) substitution at the position 234 and 235 (LALA) and the alanine (A) to asparagine (N) substitution at position 297 (N297A). In addition, the Fc fragment can be mutated to incorporate a knob-in-hole to reduce mispairing.

In accordance with one embodiment of the present disclosure, therefore, provided is a bispecific antibody, or a multi-specific antibody that incorporates the bispecific antibody, that includes an anti-5T4 portion and an anti-CD40) portion. In some embodiments, the anti-5T4 portion includes a conventional heavy chain-light chain pair. In some embodiments, the anti-CD40) portion includes at least two VHH antibodies fused in series. In some embodiments, the anti-CD40 portion includes at least three VHH antibodies fused in series. In some embodiments, the peptide chain that includes the at least two or three VHH antibodies is fused to the N-terminus of one of the two chains of the Fc fragment.

In some embodiments, the bispecific antibody has a single binding site for 5T4. In some embodiments, the bispecific antibody has two, or three, or more binding sites for CD40). In some embodiments, the Fc fragment is mutated to be deprived of ADCC/CDC activities or prevented from binding to FcγR. Example mutations include leucine (L) to alanine (A) substitution at the position 234 and 235 (LALA) and the alanine (A) to asparagine (N) substitution at position 297 (N297A). In addition, the Fc fragment can be mutated to incorporate a knob-in-hole to reduce mispairing.

In some embodiments, provided is a bispecific antibody, or a multi-specific antibody that incorporates the bispecific antibody, that includes an anti-5T4 portion and an anti-CD40 portion. In some embodiments, the anti-5T4 portion includes a conventional Fab antibody. In some embodiments, the anti-CD40 portion includes two separate VHH antibodies, each of which is fused to the C-terminus of one of the two chains of the Fc fragment.

In some embodiments, the bispecific antibody has two binding sites for 5T4 and two binding sites for CD40. In some embodiments, the Fc fragment is mutated to be deprived of ADCC/CDC activities or prevented from binding to FcγR. Example mutations include leucine (L) to alanine (A) substitution at the position 234 and 235 (LALA) and the alanine (A) to asparagine (N) substitution at position 297 (N297A). In addition, the Fc fragment can be mutated to incorporate a knob-in-hole to reduce mispairing.

Example sequences for the anti-5T4 antibodies and fragments and for the anti-CD40 VHH antibodies are provided throughout the disclosure and incorporated here.

### Chimeric Antigen Receptors (CAR)

Also provided, are chimeric antigen receptor (CAR) that includes a nanobody of the present disclosure. In the CAR, the nanobody can serve as the antigen recognition domain. In addition, in some embodiments, the CAR also includes an extracellular hinge region, a transmembrane domain, and an intracellular T-cell signaling domain.

The hinge, also called a spacer, is a small structural domain that sits between the antigen recognition region and the cell's outer membrane. A suitable hinge enhances the flexibility of the scFv receptor head, reducing the spatial constraints between the CAR and its target antigen. Example hinge sequences are based on membrane-proximal regions from immune molecules such as IgG, CD8, and CD28.

The transmembrane domain is a structural component, consisting of a hydrophobic alpha helix that spans the cell membrane. It anchors the CAR to the plasma membrane, bridging the extracellular hinge and antigen recognition domains with the intracellular signaling region. Typically, the transmembrane domain from a membrane-proximal component of the endodomain can be used, such as the CD28 transmembrane domain.

The intracellular T-cell signaling domain lies in the receptor's endodomain, inside the cell. After an antigen is bound to the external antigen recognition domain, CAR receptors cluster together and transmit an activation signal. Then the internal cytoplasmic end of the receptor perpetuates signaling inside the T cell. To mimic this process, CD3-zeta's cytoplasmic domain is commonly used as the main CAR endodomain component.

T cells also require co-stimulatory molecules in addition to CD3 signaling in order to persist after activation. In some embodiments, the endodomains of CAR receptor also includes one or more chimeric domains from co-stimulatory proteins, such as CD28, CD27, CD134 (OX40), and CD137 (4-1BB).

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

In some embodiments, the polynucleotide is an mRNA molecule. In some embodiments, the mRNA can be introduced into a target cell for expressing the antibody or fragment thereof.

mRNAs may be synthesized according to any of a variety of known methods. For example, the mRNAs may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application.

In some embodiments, for the preparation of antibody-coding mRNA, a DNA template is transcribed in vitro. A suitable DNA template typically has a promoter, for example a T3, T7 or SP6 promoter, for in vitro transcription, followed by desired nucleotide sequence for desired antibody encoding (e.g., heavy chain or light chain encoding) mRNA and a termination signal.

Desired antibody encoding (e.g., heavy chain or light chain encoding) mRNA sequence may be determined and incorporated into a DNA template using standard methods. For example, starting from a desired amino acid sequence (e.g., a desired heavy chain or light chain sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

The mRNA may be synthesized as unmodified or modified mRNA. Typically. mRNAs are modified to enhance stability. Modifications of mRNA can include, for example, modifications of the nucleotides of the RNA. A modified mRNA can thus include, for example, backbone modifications, sugar modifications or base modifications. In some embodiments, antibody encoding mRNAs (e.g., heavy chain and light chain encoding mRNAs) may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2.2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 13-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g. from the U.S. Pat. Nos. 4,373.071, 4,401,796, 4,415,732, 4,458,066, 4,500,707, 4,668,777, 4,973,679, 5,047,524, 5,132,418, 5,153,319, 5,262,530 and 5,700,642, the disclosure of which is included here in its full scope by reference.

In some embodiments, the mRNAs (e.g., heavy chain and light chain encoding mRNAs) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

In some embodiments, the mRNAs (e.g., heavy chain and light chain encoding mRNAs) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, the mRNAs (e.g., heavy chain and light chain encoding mRNAs) may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoally luridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate. N1-methyladenosine 5′-triphosphate. N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, the mRNAs (e.g., heavy chain and light chain encoding mRNAs) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A.G(5′)ppp(5)A and G(5)ppp(5′)G.

In some embodiments, the mRNAs (e.g., heavy chain and light chain encoding mRNAs) include a 3′ poly(A) tail structure. A poly-A tail on the 3′ terminus of mRNA typically includes about 10 to 300 adenosine nucleotides (e.g., about 10 to 200 adenosine nucleotides, about 10 to 175 adenosine nucleotides, about 10 to 150 adenosine nucleotides, about 10) to 125 adenosine nucleotides, 10 to 100 adenosine nucleotides, about 10 to 75 adenosine nucleotides, about 20) to 70 adenosine nucleotides, or about 20 to 60 adenosine nucleotides). In some embodiments, antibody encoding mRNAs (e.g., heavy chain and light chain encoding mRNAs) include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, the mRNAs (e.g., heavy chain and light chain encoding mRNAs) include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length (e.g., about 50 and 400 nucleotides in length, about 50 and 300 nucleotides in length, about 50 and 200 nucleotides in length, or about 50 and 100 nucleotides in length).

In some embodiments, a 5′ region of an mRNA (e.g., heavy chain and light chain encoding mRNAs) includes a sequence encoding a signal peptide, such as those described herein. In particular embodiments, a signal peptide derived from human growth hormone (hGH) is incorporated in the 5′ region. Typically, a signal peptide encoding sequence is linked, directly or indirectly, to the heavy chain or light chain encoding sequence at the N-terminus.

The present technology may be used to deliver any antibody known in the art and antibodies that can be produced against desired antigens using standard methods. The present invention may be used to deliver monoclonal antibodies, polyclonal antibodies, antibody mixtures or cocktails, human or humanized antibodies, chimeric antibodies, or bispecific antibodies.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. No. 6,150,584; 6,458,592; 6,420,140 which are incorporated by reference in their entireties.

In certain embodiments, the prepared antibodies will not elicit a deleterious immune response in the animal to be treated, e.g., in a human. In one embodiment, antigen-binding polypeptides, variants, or derivatives thereof of the disclosure are modified to reduce their immunogenicity using art—recognized techniques. For example, antibodies can be humanized, primatized, deimmunized, or chimeric antibodies can be made. These types of antibodies are derived from a non-human antibody, typically a murine or primate antibody. that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. USA 57:6851-6855 (1984); Morrison et al., Adv. Immunol. 44:65-92 (1988); Verhoeven et al., Science 239:1534-1536 (1988); Padlan, Molec. Immun. 25:489-498 (1991); Padlan, Molec. Immun. 31:169-217 (1994), and U.S. Pat. Nos. 5,585,089, 5,693,761, 5,693,762, and 6.190,370, all of which are hereby incorporated by reference in their entirety.

De-immunization can also be used to decrease the immunogenicity of an antibody. As used herein, the term “de-immunization” includes alteration of an antibody to modify T-cell epitopes (see. e.g., International Application Publication Nos.: WO/9852976 A1 and WO/0034317 A2). For example, variable heavy chain and variable light chain sequences from the starting antibody are analyzed and a human T-cell epitope “map” from each V region showing the location of epitopes in relation to complementarity-determining regions (CDRs) and other key residues within the sequence is created. Individual T-cell epitopes from the T-cell epitope map are analyzed in order to identify alternative amino acid substitutions with a low risk of altering activity of the final antibody. A range of alternative variable heavy and variable light sequences are designed comprising combinations of amino acid substitutions and these sequences are subsequently incorporated into a range of binding polypeptides. Typically, between 12 and 24 variant antibodies are generated and tested for binding and/or function. Complete heavy and light chain genes comprising modified variable and human constant regions are then cloned into expression vectors and the subsequent plasmids introduced into cell lines for the production of whole antibody. The antibodies are then compared in appropriate biochemical and biological assays, and the optimal variant is identified.

The binding specificity of antigen-binding polypeptides of the present disclosure can be determined by in vitro assays such as immunoprecipitation, radioimmunoassay (RIA) or enzyme-linked immunoabsorbent assay (ELISA).

### Treatment of Tumors, Particularly Cold Tumors

As described herein, the antibodies, variants or derivatives of the present disclosure may be used in certain treatment and diagnostic methods.

The present disclosure is further directed to antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. In some embodiments, a tumor antigen (e.g., Claudin 18.2) is overexpressed in tumor cells. Accordingly, in some embodiments, provided are methods for treating a cancer in a patient in need thereof. The method, in one embodiment, entails administering to the patient an effective amount of an antibody of the present disclosure. In some embodiments, at least one of the cancer cells (e.g., stromal cells) in the patient expresses, over-express, or is induced to express the tumor antigen. Induction of a gene expression, for instance, can be done by administration of a tumor vaccine or radiotherapy.

Tumors that can be suitably treated include those of bladder cancer, non-small cell lung cancer, renal cancer, breast cancer, urethral cancer, colorectal cancer, head and neck cancer, squamous cell cancer, Merkel cell carcinoma, gastrointestinal cancer, stomach cancer, esophageal cancer, ovarian cancer, renal cancer, and small cell lung cancer. Accordingly, the presently disclosed antibodies can be used for treating any one or more such cancers.

In some embodiments, the tumors being treated are those that are particularly challenging to treat with conventional immuno-oncological therapies, such as with antibodies targeting immune checkpoints (ICPs). Sometimes, such tumors are referred to as “cold tumors” or “nonimmunogenic tumors.” CD40 activation can convert so-called cold tumors (nonimmunogenic tumors) to hot ones. In some embodiments, accordingly, the present disclosure provides methods and uses for treating cold tumors with the antibodies disclosed herein.

In some embodiments, a nonimmunogenic tumor is one that is not infiltrated with T cells, or that is deficient in T cell filtration, in antigen presenting cells (APCs), or in T cell activation, or has deficit in T cell homing into the tumor bed. All of prostate cancer, pancreatic cancer, and leukemia are nonimmunogenic. The vast majority of breast cancer (95%), colorectal cancer (95%), gastric cancer (87%), head and neck cancer (84%), liver cancer (83%), esophageal cancer (86%), cervical cancer (87%), and thyroid cancer (87%) are also nonimmunogenic. In addition, 83% of lung cancer, 79% of bladder cancer, 77% of kidney cancer, 70% uterus cancer, and 66% melanoma are also nonimmunogenic.

Identification of nonimmunogenic, or cold tumors can also be made with measurements of type, density and location of immune cells within the tumors. For instance, Galon and Bruni (Nature Reviews Drug Discovery volume 18, pages 197-218 (2019)) describes a standardized scoring system, Immunoscore, based on the quantification of two lymphocyte populations (CD40 and CD8), e.g., in resected tissues, for guided stratification of hot and cold tumors. The Immunoscore ranges from Immunoscore 0 (10, for low densities, such as absence of both cell types in both regions) to 14 (high immune cell densities in both locations). By classifying cancers according to their immune infiltration, the scoring system provides an immune-based classification of tumors, including a definition of “hot” (highly infiltrated, Immunoscore 14) and “cold” (non-infiltrated, Immunoscore 10) tumors.

In some embodiments, the tumor is resistant to a treatment with immune checkpoint inhibitors, such as PD-L1 inhibitors, PD-1 inhibitors, CTLA-4 inhibitors, or the combinations thereof. In some embodiments, the cancer is prostate cancer, pancreatic cancer, or leukemia. In some embodiments, the cancer is breast cancer, colorectal cancer, gastric cancer, head and neck cancer, liver cancer, esophageal cancer, cervical cancer, or thyroid cancer. In some embodiments, the cancer is lung cancer, bladder cancer, kidney cancer, uterus cancer, or melanoma.

In some embodiments, a patient that is treated with a CD40 agonist antibody (or multi-specific antibody) of the present disclosure is further treated with a second anticancer agent. In some embodiment, the second anticancer agent is an immune checkpoint inhibitor, such an antibody specific to PD-1, PD-L1, or CTLA-4, without limitation. In some embodiments, the second anticancer agent is administered together with the CD40 agonist antibody (or multi-specific antibody) of the present disclosure. In some embodiments, the second anticancer agent is administered before, or after the CD40 agonist antibody (or multi-specific antibody) of the present disclosure.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibodies, variants or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antibodies, polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

### Compositions

The present disclosure also provides pharmaceutical compositions. Such compositions comprise an effective amount of an antibody, and an acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1: Generation of VHH Antibodies against Human CD40

This example describes generations of single domain (VHH) antibodies against the human CD40 protein.

Immunization: To generate VHH antibodies to human CD40, two alpaca were immunized with the human CD40 protein. After 4 rounds of immunization, the serum of immunized alpaca was subjected to antibody titer evaluation by ELISA.

Immunized library construction: The phage library was constructed using phagemid vectors which consisted of the VHH gene fragments that were amplified from PBMC of CD40 immunized alpaca. The antibody format is VHH fragment in phage display library.

Four immunized libraries were generated from PBMC of different alpaca at different immunization rounds. The size of each library was more than 1×108 and the sequence diversity was analyzed as follows. From each library, 24 or 48 clones were picked and further sequenced. Sequence showed enough diversity in CDRs for these four libraries.

Phage Panning and Clone Selection: The CD40 protein was used as antigen for phage library panning.

Phage library solution panning against human CD40: The bound phages were eluted with Gly-Hcl. The resulting phage was output 1. The bound phages were incubated with SS320 cells and plated on 2YT plates for next round of panning screening. There were total 3 rounds of panning screening. The phage ELISA of output 1, output 2 and output 3 showed enriched CD40 binders post three rounds of screening.

The single clones were picked from output 2 and output 3 phages. The phages of these clones were subjected to antigen binding ELISA. The clones that showed good binding potency were selected for subsequent sequencing.

Thirteen candidate sequences were cloned into the PcDNA 3.4 vector and expressed in 293F cells. The monoclonal antibodies were purified from the culture supernatant by protein G. The purified antibodies were subjected to ELISA binding evaluation on CD40-His protein.

The amino acid sequences of the single variable domains of 42p155, 2p834, 2p931, 42p655, 2p1294, 2p957, 42p495, 3p78, 2p415, 2p442, 2p551, 2p80 and 2p1 130 are listed in Table 1 and 1A-1E below.

TABLE 1
Variable domain sequences

| Name | Sequence | SEQ ID NO: |
|---|---|---|
| 42p155 | PSQLVESGGGLVQPGGSLRLSCAASGFAFRSYTMSWVRQAPGKGLEWVSTIT | 1 |
| | HNGAITTYAESAQGRFTISRDNAKNTLYLQMNSLKPEDTAVYYCAQGGGSNY | |
| | YRESWGQGTQVTVSS | |
| 2p834 | QSQLVESGGGLVQPGGSLRLSCAASGFAFRRYTMSWVRQAPGKGLEWVSTIT | 2 |
| | HNGSITTYAESAQGRFTISRDNAKNTLYLQMNSLKPEDTAVYYCAQGGGSNY | |
| | YRESWGQGTQVTVSS | |
| 2p931 | AVQLVESGGGLVQPGGSLRLSCAASGFSERNYMMNWVRQAPGKGLECLSSIT | 3 |
| | SAGDITSYTESVKGRFTISRDNAKNTLYLQMNSLKPEDTAVYYCAQGGCGNY | |
| | YRESWGQGTQVTVSS | |
| 42p655 | AVQLVESGGGLVQPGGSLRLSCAASGFAFRRYTMSWVRQAPGKGLEWVSSIT | 4 |
| | DNGSITTYAESAQGRFTISRDNAKNTLYLQMNSLKPEDTAVYHCAQGGGSNY | |
| | YRESWGQGTQVTVSS | |
| 2p1294 | QVQLVESGGGLVQPGGSLRLSCAASGESFRRYTMSWVRQAPGKGLEWVSAIS | 5 |
| | DNGAITTYTESAQGRFTISRDNAKNTLYLQMNSLKPEDTAVYYCAQGGGSNY | |
| | YRESWGQGTQVTVSS | |
| 2p957 | PVQLVESGGGLVQPGGSLRLSCAASGFSFRNYIMSWVRQAPGKGLEWLSSIT | 6 |
| | NSGGITSYTESVKGRFTISRDNAKNTLYLQMNSLKPEDTAVYYCAQGGSDNY | |
| | YRGSWGQGTQVTVSS | |
| 42p495 | PVQLVESGGNLVQPGGSLRLSCVAPGFTSRNSAMSWVRQAPGKGLEWVSTIY | 7 |
| | SGKSNTDYADSVKGRFTISTDNAKNTLYLHMNKLKSEDTAVYYCAKGAASDW | |
| | YVPRDYWGQGTQVTVSS | |
| 3p78 | PVQLVESGGGLVQPGGSLRLSCAASGFTERNYAMSWVRQAPGKGLEWVSTIT | 8 |
| | HNGAITTYAESAQGRFTISRDNAKNTLYLQMNSLKPEDTAVYYCAQGGGSNY | |
| | YRESWGQGTQVTVSS | |
| 42p415 | QLHFVESGGGLVQPGGSLRLSCAASGFTLDHYAMGWFRQAPGKEREGVSCIS | 9 |
| | GGGSTRYADSAKGRFTISRDNAKNTVYLQMNSLTPEDTAVYYCAAARLLSRN | |
| | CVPRDSGSWGQGTQVTVSS | |
| 2p442 | QVQLVESGGGLVQPGGSLRLSCVASGFTLDYFAIGWFRQAPGKEREGVSCIS | 10 |
| | GGGSTRYADSVKGRFTISRDNAKNTVYLQMNSLTPEDTAVYYCAAARLLSRN | |
| | CVPRDSGSWGQGTQVTVSS | |
| 2p551 | QLHEVESGGGLVQPGGSLRLSCAASGFTLDYYAIGWFRQAPGKEREGVSCIS | 11 |
| | GGGSTRYADSVKGRFTISRDNAKNTVYLQMNSLTPEDTAVYYCAAARLLSTN | |
| | CVPRDSGSWGQGTQVTVSS | |
| 2p80 | QLQLVESGGGLVQAGGSLRLSCAASGRIFSSYAMSWFRQAPGKEREFVATIG | 12 |
| | WIGENTYYADSVKGRFTISRDNAKNTLYLQMNSLKPEDTAVYYCAAGLPANR | |
| | YYDYWGQGTQVTVSS | |
| 2p1130 | EVQVVESGGGLVQPGGSLRLTCAASGFSVDDYGIGWFRQAPGREREGVACIT | 13 |
| | PNGLMMNFANTVGSVAGRFSASRDSGENTVSLQMSSLKPEDTAIYFCAHSRD | |
| | DSCRGSLSDYDDWGQGTQVTVSS | |

TABLE 1A
CDR sequences of 42p155

| Name | Sequence | SEQ ID NO: |
|---|---|---|
| 42p155-CDR1 | SYTMS | 14 |
| 42p155-CDR2 | TITHNGAITTYAESAQG | 15 |
| 42p155-CDR3 | GGGSNYYRES | 16 |

TABLE 1B
CDR sequences of 2p834

| Name | Sequence | SEQ ID NO: |
|---|---|---|
| 2p834-CDR1 | RYTMS | 17 |
| 2p834-CDR2 | TITHNGSITTYAESAQG | 18 |
| 2p834-CDR3 | GGGSNYYRES | 19 |

TABLE 1C
CDR sequences of 2p931

| Name | Sequence | SEQ ID NO: |
|---|---|---|
| 2p931-CDR1 | NYMMN | 20 |
| 2p931-CDR2 | SITSAGDITSYTESVKG | 21 |
| 2p931-CDR3 | GGCGNYYRES | 22 |

TABLE 1D
CDR sequences of 42p655

| Name | Sequence | SEQ ID NO: |
|---|---|---|
| 42p655-CDR1 | RYTMS | 23 |
| 42p655-CDR2 | SITDNGSITTYAESAQG | 24 |
| 42p655-CDR3 | GGGSNYYRES | 25 |

TABLE 1E
CDR sequences of 2p1294

| Name | Sequence | SEQ ID NO: |
|---|---|---|
| 2p1294-CDR1 | RYTMS | 26 |
| 2p1294-CDR2 | AISDNGAITTYTESAQG | 27 |
| 2p1294-CDR3 | GGGSNYYRES | 28 |

TABLE 1F
CDR sequences of 2p957

| Name | Sequence | SEQ ID NO: |
|---|---|---|
| 2p957-CDR1 | NYIMS | 29 |
| 2p957-CDR2 | SITNSGGITSYTESVKG | 30 |
| 2p957-CDR3 | GGSDNYYRGS | 31 |

TABLE 1G
CDR sequences of 42p495

| Name | Sequence | SEQ ID NO: |
|---|---|---|
| 42p495-CDR1 | NSAMS | 32 |
| 42p495-CDR2 | TIYSGKSNTDYADSVKG | 33 |
| 42p495-CDR3 | GAASDWYVPRDY | 34 |

TABLE 1H
CDR sequences of 3p78

| Name | Sequence | SEQ ID NO: |
|---|---|---|
| 3p78-CDR1 | NYAMS | 35 |
| 3p78-CDR2 | TITHNGAITTYAESAQG | 36 |
| 3p78-CDR3 | GGGSNYYRES | 37 |

TABLE 1I
CDR sequences of 2p415

| Name | Sequence | SEQ ID NO: |
|---|---|---|
| 2p415-CDR1 | HYAMG | 38 |
| 2p415-CDR2 | CISGGGSTRYADSAKG | 39 |
| 2p415-CDR3 | ARLLSRNCVPRDSGS | 40 |

TABLE 1J
CDR sequences of 2p442

| Name | Sequence | SEQ ID NO: |
|---|---|---|
| 2p442-CDR1 | YFAIG | 41 |
| 2p442-CDR2 | CISGGGSTRYADSVKG | 42 |
| 2p442-CDR3 | ARLLSRNCVPRDSGS | 43 |

TABLE 1K
CDR sequences of 2p551

| Name | Sequence | SEQ ID NO: |
|---|---|---|
| 2p551-CDR1 | YYAIG | 44 |
| 2p551-CDR2 | CISGGGSTRYADSVKG | 45 |
| 2p551-CDR3 | ARLLSTNCVPRDSGS | 46 |

TABLE 1L
CDR sequences of 2p80

| Name | Sequence | SEQ ID NO: |
|---|---|---|
| 2p80-CDR1 | SYAMS | 47 |
| 2p80-CDR2 | TIGWIGENTYYADSVKG | 48 |
| 2p80-CDR3 | GLPANRYYDY | 49 |

TABLE 1M
CDR sequences of 2p1130

| Name | Sequence | SEQ ID NO: |
|---|---|---|
| 2p1130-CDR1 | DYGIG | 50 |
| 2p1130-CDR2 | CITPNGLMMNFANTVGSVAG | 51 |
| 2p1130-CDR3 | SRDDSCRGSLSDYDD | 52 |

### Example 2: Binding Activity to CD40 Antigen

This example tested the binding activities of the antibodies towards CD40 protein.

2.1 ELISA binding to CD40

To evaluate the binding activity of clones 42p155, 2p834, 2p931, 42p655, 2p1294, 2p957, 42p495, 3p78, 2p415, 2p442, 2p551, 2p80 and 2p1130, the chimeric monospecific antibody (mAb) from these clones were subjected to ELISA test, along with selicrelumab, the clinical benchmark anti-CD40 agonist antibodies.

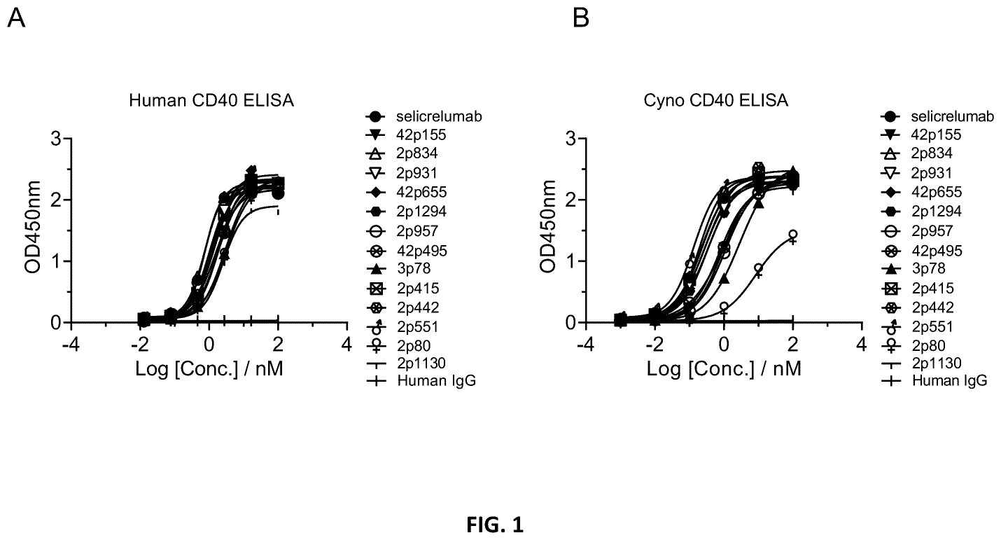

Briefly, microtiter plates were coated with human CD40-His protein at 1.0 μg/ml in PBS, 100 μl/well at 4° C. overnight, then blocked with 150 μl/well of 1% BSA. Ten-fold dilutions of 42p155, 2p834, 2p931, 42p655, 2p1294, 2p957, 42p495, 3p78, 2p415, 2p442, 2p551, 2p80 and 2p1 130 antibodies starting from 100 nM were added to each well and incubated for 1 hour at RT. The plates were washed with PBS/Tween and then incubated with Anti-Human IgG (H&L) (GOAT) Antibody Peroxidase Conjugated for 30 mins at RT. After washing, the plates were developed with TMB substrate and analyzed by spectrophotometer at OD 450 nm. As shown in FIG. 1A and Table 2, all these clones bound to human CD40 at high activity, and the potency are comparable to the selicrelumab.

The binding affinity of these clones to cynomolgus CD40 was also tested. Microtiter plates were coated with cynomolgus CD40-His protein at 1.0 μg/ml in PBS, 100 μl/well at 4° C. overnight, then blocked with 150 μl/well of 1% BSA. Ten-fold dilutions of 42p155, 2p834, 2p931, 42p655, 2p1294, 2p957, 42p495, 3p78, 2p415, 2p442, 2p551, 2p80 and 2p1 130 antibodies starting from 100 nM were added to each well and incubated for 1 hour at RT. The plates were washed with PBS/Tween and then incubated with Anti-Human IgG (H&L) (GOAT) Antibody Peroxidase Conjugated for 30 mins at RT. After washing, the plates were developed with TMB substrate and analyzed by spectrophotometer at OD 450 nm. As shown in FIG. 1B and Table 2, all these clones bound to cynomolgus CD40 at high activity, showing good cross-reactivity between human and cynomolgus CD40 proteins.

TABLE 2
Cross species activity of the clones

EC₅₀ (nM)

| Clone | Human CD40 | Cynomolgus CD40 |
|---|---|---|
| 42p155 | 1.73 | 0.90 |
| 2p834 | 0.76 | 0.20 |
| 2p931 | 0.99 | 0.26 |
| 42p655 | 1.24 | 0.35 |
| 2p1294 | 1.16 | 0.32 |
| 2p957 | 1.80 | 1.08 |
| 42p495 | 1.52 | 1.00 |
| 3p78 | 2.95 | 2.84 |
| 2p415 | 1.27 | 0.23 |
| 2p442 | 1.95 | 0.30 |
| 2p551 | 1.40 | 0.13 |
| 2p80 | 3.08 | 8.29 |
| 2p1130 | 2.56 | 0.73 |

—: No binding

### 2.2 Binding to Cell Surface CD40

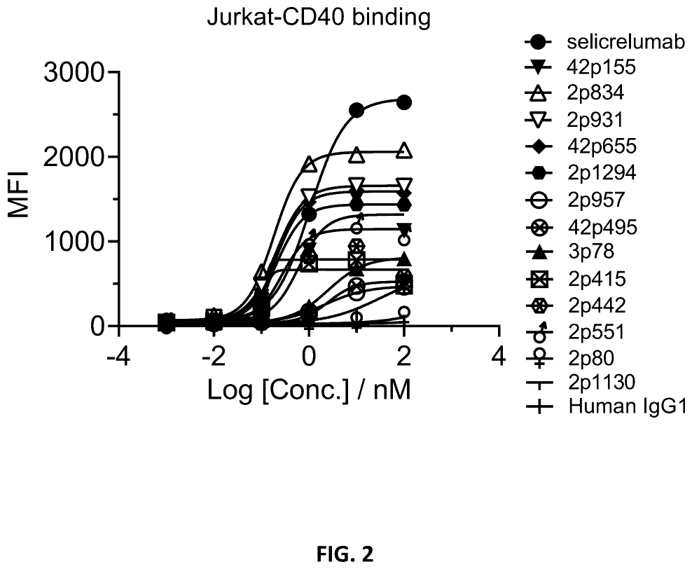

To evaluate their binding affinity to cell surface CD40, chimeric anti-CD40 monospecific antibodies were tested in Jurkat cell lines that overexpressed CD40 by FACS. A total number of 1×10⁵ Jurkat-CD40 cells in each well were incubated with 10-fold serially diluted antibodies starting from 100 nM for 30 minutes at 4° C. in FACS buffer. After wash by FACS buffer, PE conjugated-anti-human IgG antibody was added to each well and incubated at 4° C. for 30 minutes. After wash, MFI of PE was evaluated by MACSQuant Analyzer 16. As shown in FIG. 2, the tested antibodies showed concentration-dependent binding abilities to CD40.

### 2.3 Protein Full Kinetic for CD40

The binding of the antibodies, either in a monospecific antibody format (cAb) or in a bispecific format (BiAb, as shown in the FIG. 4) along with an anti-claudin 18.2 portion, to recombinant CD40 protein (human CD40-his tag) was tested with Biacore using a capture method. Bispecific antibodies with each of the 42p155, 2p834, 2p931, 42p655, 2p1294, 2p957, 42p495, 3p78, 2p415, 2p442, 2p551 and 2p1 130 clones, as well as 2p80 monospecific antibody, were captured using Protein A chip. A serial dilution of human CD40-his tag protein was injected over captured antibody for 2 mins at a flow rate of 30 μl/min. The antigen was allowed to dissociate for 6 min. All the experiments were carried out on a Biacore T200. Data analysis was carried out using the Biacore T200 evaluation software. The results are shown in Table 4 below. All antibodies exhibited moderate bindings and differentiated association/disassociation modalities.

TABLE 4
Full kinetic measured by Biacore

| | Human CD40-His | | |
|---|---|---|---|
| Abs | ka (1/Ms) | kd (1/s) | KD (M) |
| 42p155 BiAb | 4.91 × 10⁴ | 3.74 × 10⁻² | 7.60 × 10⁻⁷ |
| 2p834 BiAb | 9.27 × 10⁴ | 8.40 × 10⁻³ | 9.06 × 10⁻⁸ |
| 2p931 BiAb | 4.74 × 10⁴ | 4.09 × 10⁻³ | 8.62 × 10⁻⁸ |
| 42p655 BiAb | 9.31 × 10⁴ | 4.42 × 10⁻³ | 4.75 × 10⁻⁸ |
| 2p1294 BiAb | 1.03 × 10⁵ | 1.87 × 10⁻² | 1.82 × 10⁻⁷ |
| 2p957 BiAb | 4.64 × 10⁴ | 1.07 × 10⁻¹ | 2.31 × 10⁻⁶ |
| 42p495 BiAb | 1.27 × 10⁴ | 1.18 × 10⁻² | 9.35 × 10⁻⁷ |
| 3p78 BiAb | 4.27 × 10⁴ | 1.07 × 10⁻¹ | 2.49 × 10⁻⁶ |
| 2p415 BiAb | 5.93 × 10⁴ | 4.47 × 10⁻² | 7.54 × 10⁻⁷ |
| 2p442 BiAb | 4.37 × 10⁴ | 4.50 × 10⁻² | 1.03 × 10⁻⁶ |
| 2p551 BiAb | 3.61 × 10⁴ | 1.77 × 10⁻² | 4.92 × 10⁻⁷ |
| 2p80 cAb | 1.38 × 10³ | 2.93 × 10⁻⁴ | 2.13 × 10⁻⁷ |
| 2p1130 BiAb | 2.54 × 10⁴ | 7.05 × 10⁻² | 2.77 × 10⁻⁶ |

2.4 Cross Reactivity with OX40 and 4-1BB

To evaluate the cross reactivity of chimeric anti-CD40 antibodies to other family members of TNF receptors, the ELISA binding towards human 4-1BB and human OX40 were performed.

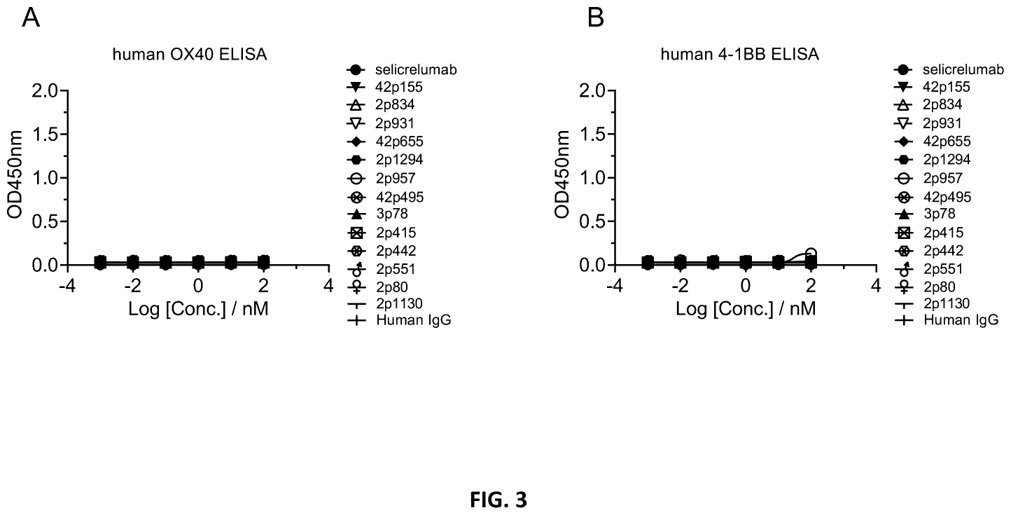

Briefly, microtiter plates were coated with human 4-1BB protein or human OX40 protein at 1 μg/ml in PBS, 100 μl/well at 4° C. overnight, then blocked with 150 μl/well of 1% BSA. Ten-fold dilutions of 42p155, 2p834, 2p931, 42p655, 2p1294, 2p957, 42p495, 3p78, 2p415, 2p442, 2p551, 2p80 and 2p1130 antibodies starting from 100 nM were added to each well and incubated for 1 hour at RT. The plates were washed with PBS/Tween and then incubated with Anti-Human IgG (H&L) (GOAT) Antibody Peroxidase Conjugated for 30 mins at RT. After washing, the plates were developed with TMB substrate and analyzed by spectrophotometer at OD 450 nm. As shown in FIG. 3, all these clones do not show cross-reactivity to human 4-1BB or human OX40.

### Example 3: The Binding Activity of Claudin18.2/CD40 Bispecific Antibodies to Human CD40 on Human Dendritic Cell and B Cell

In this example, bispecific antibodies (BiAb) that comprising anti-CD40 nanobodies and an anti-Claudin 18.2 (CLDN18.2) unit were generated and tested. Two anti-CD40 fragments with 42p155, 2p834, 2p931, 42p655, 2p1294, 2p957, 42p495, 3p78, 2p415, 2p442, 2p551, 2p80 or 2p1 130 in the VHH form (at the C-terminus) and two anti-claudin 18.2 units in the Fab form (at the N-terminus) were constructed into a 2+2 bispecific antibody format (see illustration in FIG. 4). FACS was used to evaluate the binding activity of the anti-Claudin 18.2/CD40 bispecific antibodies to CD40 on human dendritic cells (DC) and human B cells.

### 3.1 Binding Activity of the Bispecific Antibodies to Human CD40 on Human Dendritic Cell

Human DCs were induced in the presence of IL-4 and GM-CSF for 6-7 days from human CD14+ cells, which were isolated from human peripheral blood mononuclear cell (PBMC) using CD14 microbeads according to the manufacturer's protocol. Induced human DCs were first blocked with FcR block reagent (MACS) at 4° C. for 15 mins, and then incubated with 10-fold serially diluted Claudin 18.2/CD40 bispecific antibodies starting at 100 nM at 4° C. for 30 mins. PE Goat anti-Human IgG Fc Secondary Antibody (eBioscience™, Invitrogen) was added to each well and incubated at 4° C. for 30 mins. Samples were washed with FACS buffer and then incubated with APC Mouse Anti-Human CD11C (BD) at 4° C. for 30 minutes. The mean florescence intensity (MFI) of PE gated on CD11C+ cells was evaluated by MACSQuant Analyzer 16.

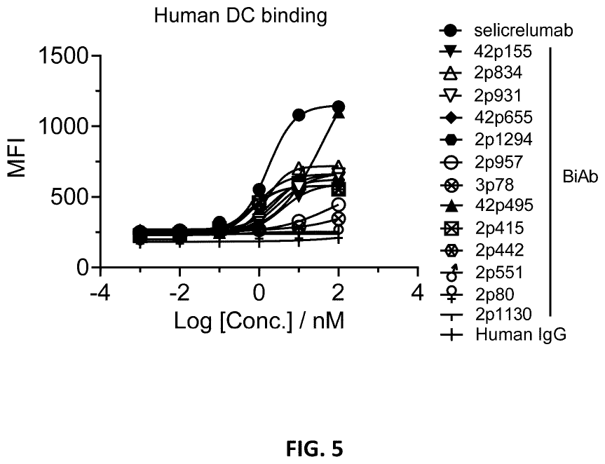

The results are shown in FIG. 5. The tested anti-CLDN18.2/CD40 bispecific antibodies showed concentration-dependent binding abilities to human DCs. The binding activity of bispecific antibodies were weaker than the reference monospecific antibodies selicrelumab.

### 3.2 Binding Activity of the Bispecific Antibodies to Human CD40 on Human B Cell

Human B cells were isolated from human PBMC using B cell isolation kit according to the manufacturer's protocol. Human B cells were first blocked with FcR block reagent (MACS) at 4° C. for 15 mins, and then incubated with 10-fold serially diluted CLDN18.2/CD40 bispecific antibodies starting at 100 nM at 4° C. for 30 mins. PE Goat anti-Human IgG Fc Secondary Antibody (eBioscience™, Invitrogen) was added to each well and incubated at 4° C. for 30 mins. Samples were washed with FACS buffer and then incubated with APC Mouse Anti-Human CD19 (BD) at 4° C. for 30 minutes. The mean florescence intensity (MFI) of PE gated on CD19+ cells was evaluated by MACSQuant Analyzer 16.

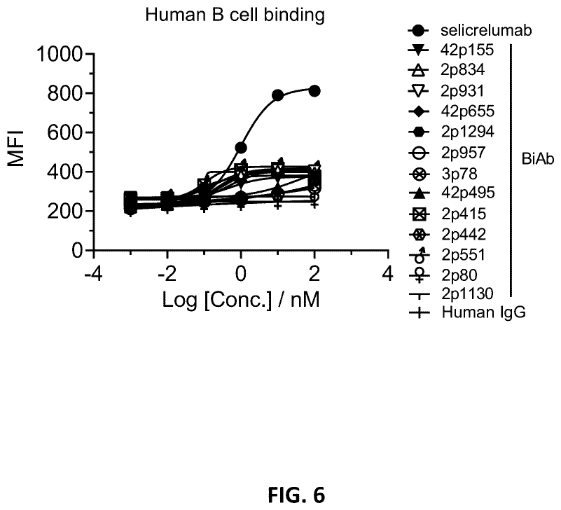

The results are shown in FIG. 6. The tested anti-CLDN18.2/CD40 bispecific antibodies showed concentration-dependent binding abilities to human B cells. The binding activity of bispecific antibodies were much weaker than the reference monospecific antibodies selicrelumab.

### Example 4. Functional Activity of CD40 Nanobodies

This example tested the functional activities of the antibodies, and shows that unlike selicrelumab, the VHH-chimeric antibodies activate CD40 signaling only in a low level.

### Cell-Line Based Functional Characterization of CD40 Monoclonal Antibodies

To assess the ability of the CD40 monoclonal antibodies to activate the CD40 signaling pathway, a commercial CD40 NF-κB luciferase reporter system was used. In this assay, H_CD40 (TNFRSF5) NFκB-Reporter Jurkat (Genomeditech, cat #GM-C09520) was used as reporter cell line. H_CD40 (TNFRSF5) NFκB-Reporter Jurkat cell line is genetically modified to stably express CD40 and luciferase downstream of a response element. Luciferase expression is induced upon antibody binding to the CD40 receptor. Briefly, reporter cells at a density of 2.5×10⁴ cells per well were cultured in a white 96-well plate. Antibodies were 10-fold serially diluted and added to a white 96-well assay plate, at a final concentration ranging from 0.001 nM to 100 nM. After 5 hours incubation at 37° C., luminescence was obtained by adding the substrate of luciferase and measured by a microplate reader. Four-parameter logistic curve analysis was performed with GraphPad software.

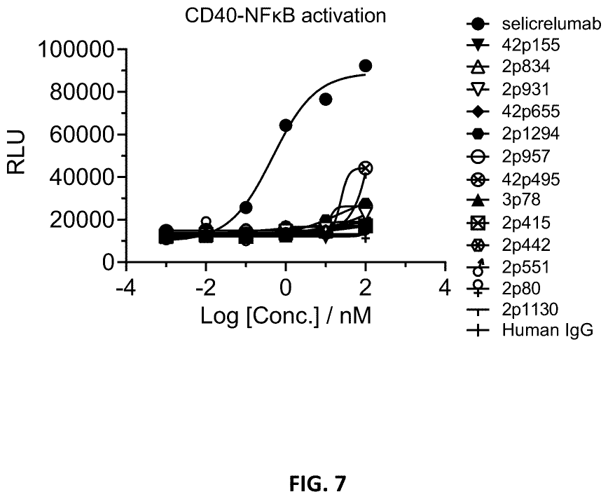

As shown in FIG. 7, the selicrelumab monoclonal antibody dose-dependently activated the CD40 signaling. The 42p155, 2p834, 2p931, 42p655, 2p1294, 2p957, 42p495, 3p78, 2p415, 2p442, 2p551, 2p80 and 2p1130 antibodies only induced CD40 signaling at a dose higher than 10 nM and their maximal RLU values were all less than half of the top value for selicrelumab in the same experimental settings.

### Example 5. Functional Activity of Anti-Claudin 18.2/CD40 Bispecific Antibodies

In this example, the activity of anti-Claudin 18.2/CD40 bispecific antibodies generated in the format shown in FIG. 4, were tested in a CD40 NF-κB luciferase reporter system, as well as to promote human dendritic cell immune response and B cell immune response.

### 5.1 Cell-Line Based Functional Characterization of Claudin 18.2-CD40 Bispecific Antibody

To assess the ability of the anti-Claudin 18.2/CD40 bispecific antibodies to activate CD40 signaling pathway, a commercial CD40 NF-κB luciferase reporter system was used. In this assay, H_CD40(TNFRSF5) NFκB-Reporter Jurkat (Genomeditech, cat #GM-C09520) was used as effector cells and CHO-K1-expressing or not expressing Claudin 18.2 as target cells. Briefly, effector cells at a density of 2.0×10⁴ cells per well were cocultured with 2.0×10⁴ target cells (E/T Ratio=1:1) in a white 96-well plate. Antibodies were 10-fold serially diluted and added to a white 96-well assay plate, at a final concentration ranging from 0.001 nM to 100 nM. After 5 hours incubation at 37° C., luminescence was obtained by adding the substrate of luciferase and measured by a microplate reader. Four-parameter logistic curve analysis was performed with GraphPad software.

As shown in FIG. 8, the selicrelumab monoclonal antibody can dose-dependently boost the CD40 signaling in both CHO-K1 and CHO-Claudin 18.2-overexpressing cells. While the activity of anti-Claudin18.2/CD40 bispecific antibodies, 42p155-BiAb, 2p834-BiAb, 2p931-BiAb, 42p655-BiAb, 2p1294-BiAb, 2p957-BiAb, 42p495-BiAb, 3p78-BiAb, 2p415-BiAb, 2p442-BiAb, 2p551-BiAb, 2p80-BiAb and 2p1130-BiAb were solely or partly dependent on the expression of Claudin 18.2 on the cells, showing much stronger activation of CD40 signaling in the presence of Claudin18.2. Moreover, the bispecific antibodies showed a variety of different CD40 activities. Based on the EC₅₀ and top value, CD40 clones were divided into four categories (Grades 1-4), as shown in Table 5.

TABLE 5
Potency Categories

| | Target cell | | |
|---|---|---|---|
| Category | C18.2− | C18.2+ | Example Antibodies |
| Grade 4 | No response | + | 2p80, 2p1130 |
| Grade 3 | Moderate response | ++++ | 2p415, 2p442, 2p551 |
| Grade 2 | Low and marginal | ++ | 2p957, 42p495, 3p78 |
| | response | | |
| Grade 1 | Minimal response | +++ | 42p155, 2p834, 2p931, |
| | | | 42p655, 2p1294 |

### 5.2 Activity of the Bispecific Antibodies to Promote Human Dendritic Cell Immune Response

To investigate the ability of the Claudin 18.2-CD40 bispecific antibodies to stimulate human dendritic cell (DC) response, IL-12 cytokine release and CD80/CD86 expression of DCs were examined.

Human DCs were obtained following the procedures specified in Example 3.1. Human DCs were used as the effector cells. CHO-K1 cells expressing Claudin 18.2 were used as the target cells. Human DCs (5×10⁴) were co-cultured with CHO-K1-Claudin 18.2 or parental CHO-K1 cells (1.5×10⁴) (E/T Ratio=˜ 3:1). Bispecific antibodies were 10-fold diluted serially and added to the culture medium at a final concentration starting from 100 nM. After incubation for 48 hours, the level of IL-12 in the culture medium was measured using IL-12/p40 (human) LANCE Ultra TR-FRET Detection Kit (PerkinElmer). Data were analyzed using a nonlinear regression, 4-parameter logistic equation.

Activation of DC leads to upregulation of costimulatory molecules CD80/86. Here. CD80/CD86 expression of DCs were examined by FACS following the staining and analysis procedures. Briefly, stimulated DCs were harvest by pipetting and washed by FACS buffer. PE Mouse anti-human CD80. BV421 Mouse anti-human CD86 and APC Mouse Anti-Human CD11C (BD) was added to each well and incubated at 4° C. for 30 minutes. After wash. MFI of PE and BV421 gated on CD11C+ cells were evaluated by MACSQuant Analyzer 16.

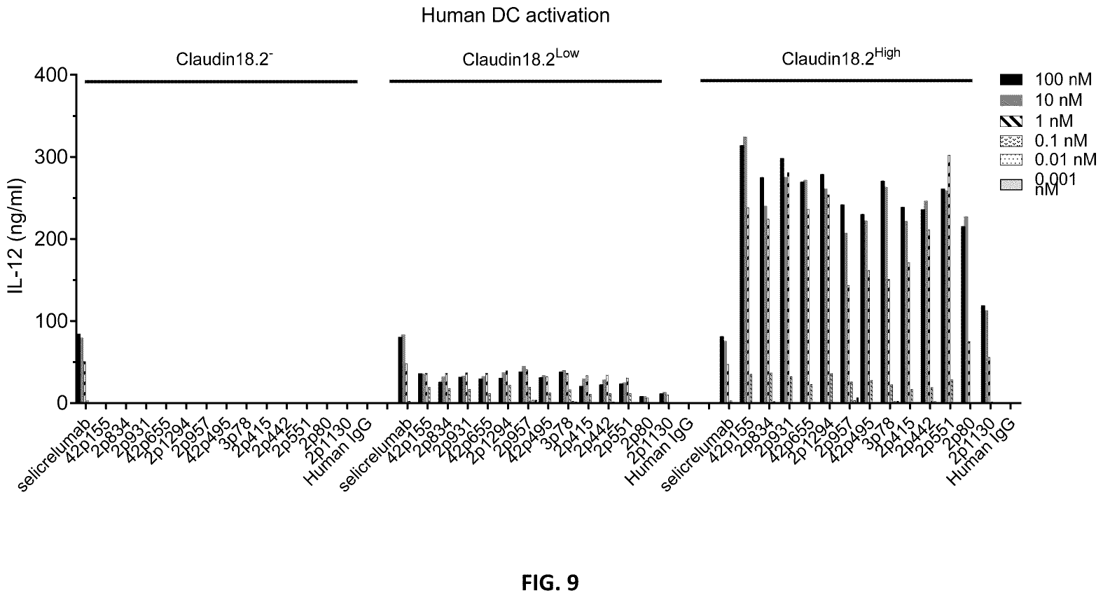

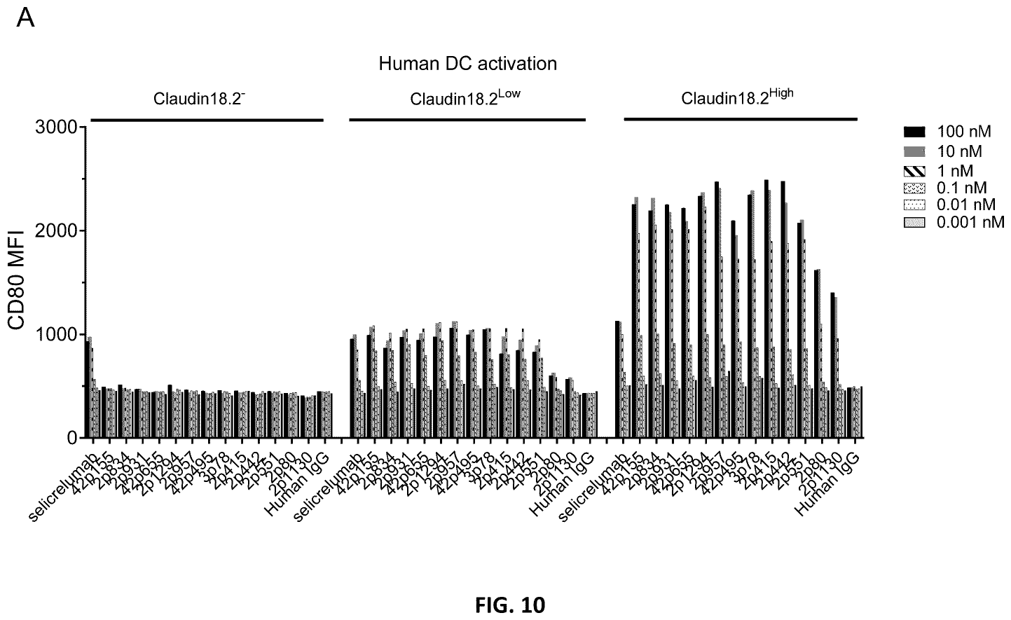

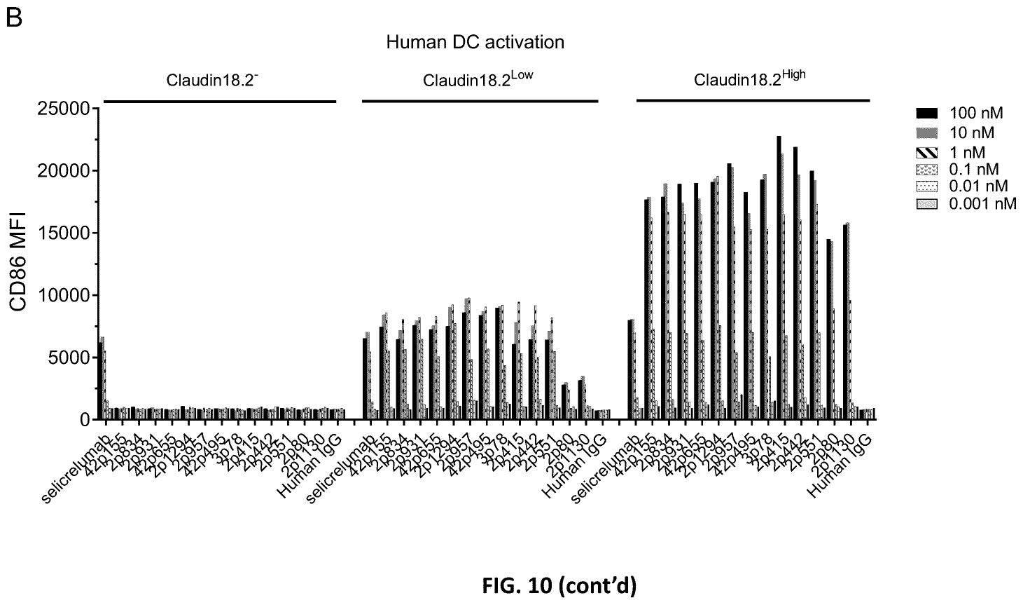

As shown in FIGS. 9 and 10, the selicrelumab monoclonal antibody can dose-dependently activate DC response (IL-12 secretion in FIG. 9 and CD80/CD86 expression in FIG. 10A-B) in both CHO-K1 and CHO-Claudin 18.2-overexpressing cells. While the bispecific antibodies could only activate DC response in the presence of Claudin 18.2 overexpressing cells. And the potency was correlated with the expression level of CLDN18.2

### 5.3 Activity of the Bispecific Antibodies to Promote Human B Cell Immune Response

To investigate the ability of the Claudin 18.2-CD40 bispecific antibodies to activate human B cell. Ki67 (cell proliferation) and CD86 expression (cell activation) of B cells were examined. Human B cells were isolated from human PBMC using B cell isolation kit according to the manufacturer's protocol. Human B cells were used as the effector cells. CHO-K1 cells expressing Claudin 18.2 were used as the target cells. Human B cells (5×10⁴) were co-cultured with CHO-K1-Claudin 18.2 or parental CHO-K1 cells (1×10⁴) (E/T Ratio=5:1). Bispecific antibodies were 10-fold serially diluted and added to the culture medium at a final concentration starting from 100 nM. After 72 hours incubation. Ki67 and CD86 expression of B cells were examined by FACS following the staining and analysis procedures. Briefly, stimulated B cells were harvest by pipetting and washed by FACS buffer. BV421 Mouse anti-human CD86 and APC Mouse Anti-Human CD19 (BD) was added to each well and incubated at 4° C. for 30 minutes. After wash, cells were fixed and permeabilized using Foxp3/Transcription factor staining buffer set (Invitrogen) and then stained Alexa FluorR 488 anti-human Ki-67 Antibody at 4° C. for 30 minutes. After wash, MFI of AF488 and BV421 gated on CD19+ cells were evaluated by MACSQuant Analyzer 16.

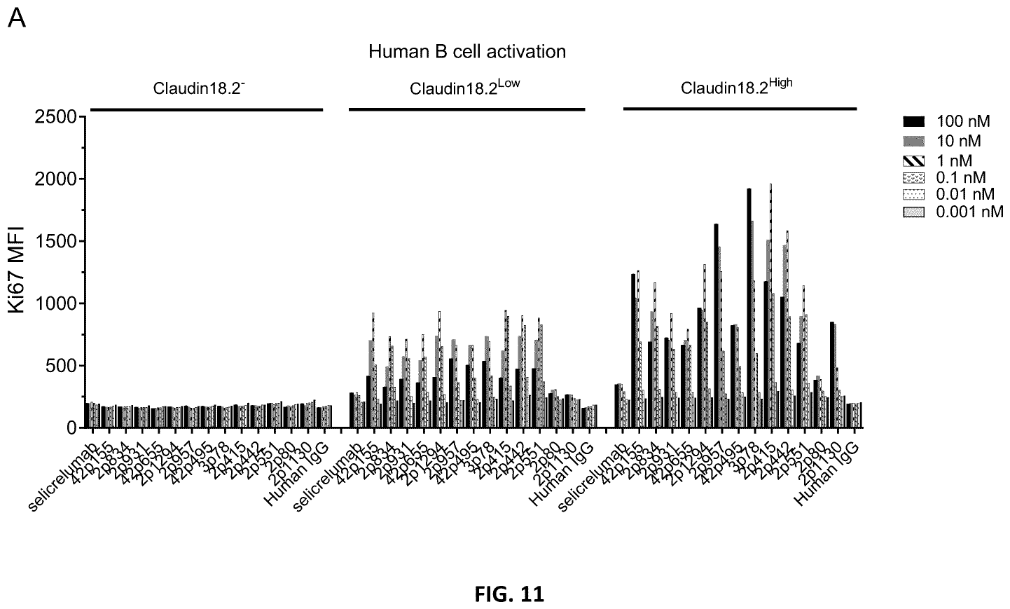

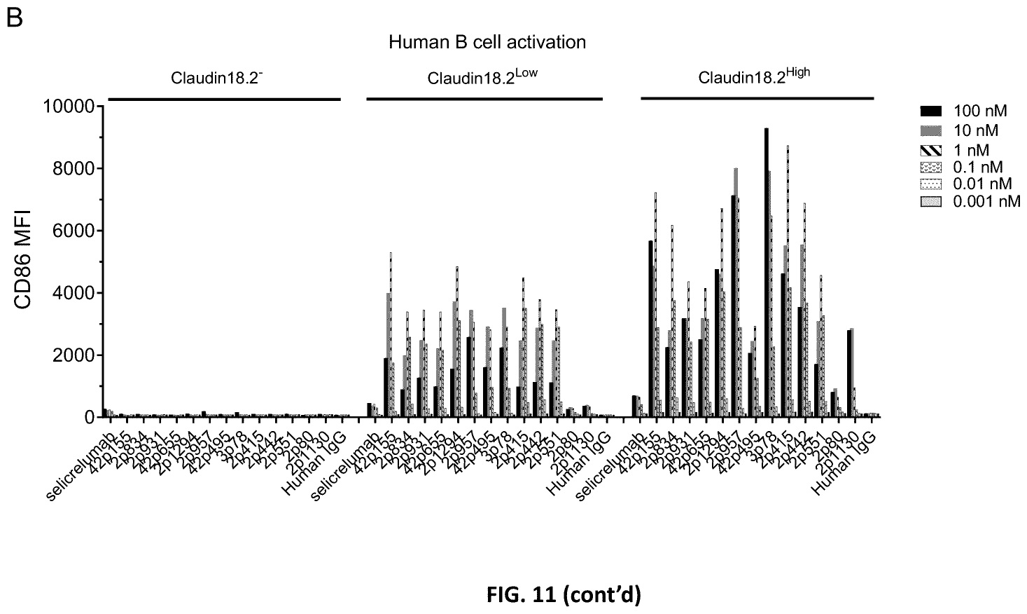

As shown in FIG. 11, the selicrelumab monoclonal antibody can dose-dependently activate B cell response (Ki67/CD86 expression in FIG. 11 A/B) in both CHO-K1 and CHO-Claudin 18.2 overexpression cells, while the bispecific antibodies could only activate B cell response in the presence of Claudin 18.2-overexpressing cells and the potency was correlated with the expression level of CLDN18.2, further demonstrated the tumor-antigen dependent nature of CD40 agonist antibodies.

### Example 6. Humanization of the CD40 VHH Antibodies

The 42p155 and 2p442 variable region genes were employed to create humanized mAbs. In the first step of this process, the amino acid sequences of the VHH of 42p155 and 2p442 were compared against the available database of human Ig gene sequences to find the overall best-matching human germline Ig gene sequences. Humanized variable domain sequences of 42p155 and 2p442 were then designed where the CDRH1, H2, and H3 onto framework sequences of their VH genes, respectively.

The amino acid sequences of some of the humanized antibodies are listed in Table 6 below.

TABLE 6-1
Humanized 42p155 antibody sequences (underlining indicates CDR; bold/italic
indicates back mutations; bold indicates PTM removal)

| | | SEQ ID |
|---|---|---|
| 42p155 | Sequence | NO: |
| VHH | PSQLVESGGGLVQPGGSLRLSCAASGFAFRSYTMSWVRQAPGKGLEW | 1 |
| | VSTITHNGAITTYAESAQGRFTISRDNAKNTLYLQMNSLKPEDTAVY | |
| | YCAQGGGSNYYRESWGQGTQVTVSS | |
| VHH grafted | EVQLVESGGGLVQPGGSLRLSCAASGFAFRSYTMSWVRQAPGKGLEW | 53 |
| | VSTITHNGAITTYAESAQGRFTISRDNSKNTLYLQMNSLRAEDTAVY | |
| | YCAKGGGSNYYRESWGQGTQVTVSS | |
| VHHz2 | EVQLVESGGGLVQPGGSLRLSCAASGFAFRSYTMSWVRQAPGKGLEW | 54 |
| | VSTITHNGAITTYAESAQGRFTISRDNSKNTLYLQMNSLRAEDTAVY | |
| | YCAQGGGSNYYRESWGQGTQVTVSS | |
| VHHz2 NA | EVQLVESGGGLVQPGGSLRLSCAASGFAFRSYTMSWVRQAPGKGLEW | 55 |
| | VSTITHNAAITTYAESAQGRETISRDNSKNTLYLQMNSLRAEDTAVY | |
| | YCAQGGGSNYYRESWGQGTQVTVSS | |
| VHHz2 QG | EVQLVESGGGLVQPGGSLRLSCAASGFAFRSYTMSWVRQAPGKGLEW | 56 |
| | VSTITHQGAITTYAESAQGRFTISRDNSKNTLYLQMNSLRAEDTAVY | |
| | YCAQGGGSNYYRESWGQGTQVTVSS | |
| VHHz3 | EVQLVESGGGLVQPGGSLRLSCAASGFAFRSYTMSWVRQAPGKGLEW | 57 |
| | VSTITHNGAITTYAESAQGRETISRDNSKNTLYLQMNSLRPEDTAVY | |
| | YCAQGGGSNYYRESWGQGTQVTVSS | |
| VHHz3 NA | EVQLVESGGGLVQPGGSLRLSCAASGFAFRSYTMSWVRQAPGKGLEW | 58 |
| | VSTITHNAAITTYAESAQGRFTISRDNSKNTLYLQMNSLRPEDTAVY | |
| | YCAQGGGSNYYRESWGQGTQVTVSS | |
| VHHz3 QG | EVQLVESGGGLVQPGGSLRLSCAASGFAFRSYTMSWVRQAPGKGLEW | 59 |
| | VSTITHQGAITTYAESAQGRFTISRDNSKNTLYLQMNSLRPEDTAVY | |
| | YCAQGGGSNYYRESWGQGTQVTVSS | |
| VHHz4 | PSQLVESGGGLVQPGGSLRLSCAASGFAFRSYTMSWVRQAPGKGLEW | 60 |
| | VSTITHNGAITTYAESAQGRFTISRDNSKNTLYLQMNSLRPEDTAVY | |
| | YCAQGGGSNYYRESWGQGTQVTVSS | |
| VHHz4 NA | PSQLVESGGGLVQPGGSLRLSCAASGFAFRSYTMSWVRQAPGKGLEW | 61 |
| | VSTITHNAAITTYAESAQGRFTISRDNSKNTLYLQMNSLRPEDTAVY | |
| | YCAQGGGSNYYRESWGQGTQVTVSS | |
| VHHz4 QG | PSQLVESGGGLVQPGGSLRLSCAASGFAFRSYTMSWVRQAPGKGLEW | 62 |
| | VSTITHQGAITTYAESAQGRFTISRDNSKNTLYLQMNSLRPEDTAVY | |
| | YCAQGGGSNYYRESWGQGTQVTVSS | |

TABLE 6-1A
CDR sequences of 42p155 (with PTM-derisked versions)

| Name | Sequence | SEQ ID NO: |
|---|---|---|
| 42p155-CDR1 | SYTMS | 14 |
| 42p155-CDR2 | TITHNGAITTYAESAQG | 15 |
| | TITHNAAITTYAESAQG | 63 |
| | TITHQGAITTYAESAQG | 64 |
| 42p155-CDR3 | GGGSNYYRES | 16 |

TABLE 6-2
Humanized 2p442 antibody sequences (underlining indicates CDR; bold/italic
indicates back mutations)

| | | SEQ ID |
|---|---|---|
| 2p442 | Sequence | NO: |
| VHH | QVQLVESGGGLVQPGGSLRLSCVASGFTLDYFAIGWERQAPGKEREG | 10 |
| | VSCISGGGSTRYADSVKGRFTISRDNAKNTVYLQMNSLTPEDTAVYY | |
| | CAAARLLSRNCVPRDSGSWGQGTQVTVSS | |
| VHH grafted | QVQLVESGGGLVKPGGSLRLSCAASGFTFSYFAIGWIRQAPGKGLEW | 69 |
| | VSCISGGGSTRYADSVKGRFTISRDNAKNSLYLQMNSLRAEDTAVYY | |
| | CARARLLSRNCVPRDSGSWGQGTLVTVSS | |
| VHHz8 | QVQLVESGGGLVKPGGSLRLSCAASGFTFDYFAIGWFRQAPGKGREG | 70 |
| | VSCISGGGSTRYADSVKGRFTISRDNAKNSVYLQMNSLRPEDTAVYY | |
| | CARARLLSRNCVPRDSGSWGQGTLVTVSS | |
| VHHz9 | QVQLVESGGGLVKPGGSLRLSCAASGFTFDYFAIGWFRQAPGKELEG | 71 |
| | VSCISGGGSTRYADSVKGRFTISRDNAKNSVYLQMNSLRPEDTAVYY | |
| | CARARLLSRNCVPRDSGSWGQGTLVTVSS | |
| VHHz10 | QVQLVESGGGLVKPGGSLRLSCAASGFTFDYFAIGWFRQAPGKELEG | 72 |
| | VSCISGGGSTRYADSVKGRFTISRDNAKNSVYLQMNSLRPEDTAVYY | |
| | CAAARLLSRNCVPRDSGSWGQGTLVTVSS | |

The back mutations of 42p155 include IP, 2S, 88P, and 98Q. More specifically, VHH-v2 included back mutations 98Q; VHH-v3 included back mutations 88P and 98Q; VHH-v4 included back mutations 1P, 2S, 88P, and 98Q.

The back mutations of 2p442 include 30D, 37F, 44E, 45R, 47G, 78V, 87P and 97A. More specifically, VHH-v8 included back mutations 30D, 37F, 45R, 47G, 78V and 87P; VHH-v9 included back mutations 30D, 37F, 44E, 47G, 78V and 87P; VHH-v10 included back mutations 30D, 37F, 44E, 47G, 78V, 87P and 97A.

The humanized VHH genes were cloned into pcDNA3.4 vectors and transfected into 293F cells for further analysis.

### Example 7: Antigen Binding Properties of the Humanized Antibodies

### 7.1 Full Kinetic Affinity of Humanized Antibodies by Biacore and Octet

The binding of the humanized 42p155 and 2p442 antibodies to recombinant human CD40 protein (human CD40-his tag) was tested by Biacore and Octet, respectively, using a capture method.

For humanized 42p155 monoclonal antibodies, 42p155z2 and 42p155z3 were captured using Protein A chip. A serial dilution of human CD40-his tag protein was injected over captured antibody for 3 mins at a flow rate of 10 μl/min. The antigen was allowed to dissociate for 6 min. All the experiments were carried out on a Biacore T200. Data analysis was carried out using Biacore T200 evaluation software.

For humanized 2p442 monoclonal antibodies, 2p442z8, 2p442z9 and 2p442z10 were captured using AHC biosensor. A serial dilution of human CD40-his tag protein was incubated with captured antibody for 5 mins. The antigen was allowed to dissociate for 10 min. All the experiments were carried out on an Octet RED96e. Data analysis was carried out using Octet Analysis Studio 12.2 software.

The results are shown in Table 7 below. All humanized antibodies exhibited moderate bindings, and comparable to parental chimeric antibody.

TABLE 7-1
Full kinetic of humanized 42p155 by Biacore

| | CD40-His | | |
|---|---|---|---|
| Abs | ka (1/Ms) | kd (1/s) | KD (M) |
| 42p155 | 5.19 × 10⁵ | 4.51 × 10⁻² | 8.69 × 10⁻⁸ |
| 42p155z2 | 1.98 × 10⁵ | 3.68 × 10⁻² | 1.86 × 10⁻⁷ |
| 42p155z3 | 2.87 × 10⁵ | 5.55 × 10⁻² | 1.93 × 10⁻⁷ |

TABLE 7-2
Full kinetic of humanized 2p442 by Octet

| | CD40-His | | |
|---|---|---|---|
| Abs | ka (1/Ms) | kd (1/s) | KD (M) |
| 2p442 | 5.34 × 10⁵ | 2.69 × 10⁻² | 5.04 × 10⁻⁸ |
| 2p442z8 | 2.10 × 10⁵ | 1.19 × 10⁻² | 5.67 × 10⁻⁸ |
| 2p442z9 | 3.57 × 10⁵ | 1.25 × 10⁻² | 3.48 × 10⁻⁸ |
| 2p442z10 | 4.85 × 10⁵ | 3.37 × 10⁻² | 6.96 × 10⁻⁸ |

7.2 Binding of humanized 2p442 antibodies to cell surface CD40

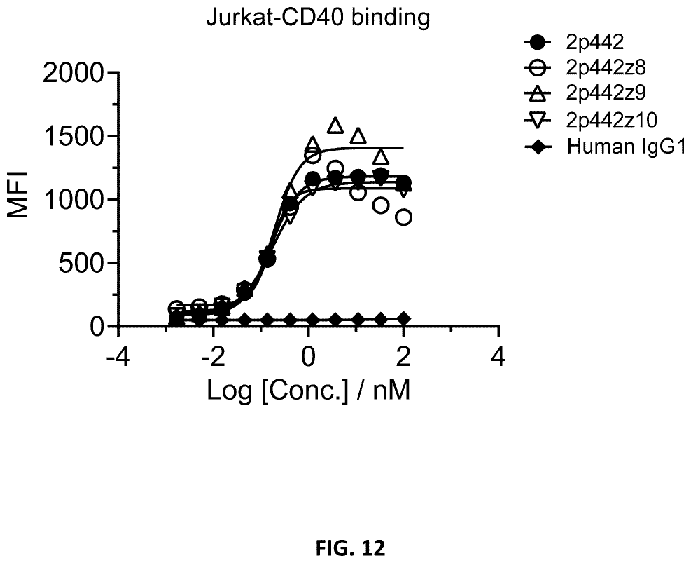

To evaluate the binding affinity of humanized 2p442 monoclonal antibodies to cell surface CD40, 2p442z8, 2p442z9, 2p442z10 and parental 2p442 were tested in Jurkat cell lines that overexpressed CD40 by FACS. A total number of 1×10⁵ Jurkat-CD40 cells in each well were incubated with 3-fold serially diluted antibodies starting from 100 nM for 30 minutes at 4° C. in FACS buffer. After wash by FACS buffer, PE conjugated-anti-human IgG antibody was added to each well and incubated at 4° C. for 30 minutes. After wash, MFI of PE was evaluated by MACSQuant Analyzer 16. As shown in FIG. 12, humanized 2p442 antibodies showed comparable binding activity to parental chimeric antibody.

### Example 8. Functional Activity of Humanized Bispecific Antibodies

Anti-Claudin 18.2/CD40 bispecific antibodies were prepared with the humanized CD40 nanobodies and tested in this example, 2p834z2, 2p834z3, and 2p834z4 used the same human FRs as 42p155z2, 42p155z3, and 42p155z4, respectively, to generate Claudin 18.2-2p-834 humanized bispecific antibodies. The amino acid sequences of the humanized 2p834 antibodies are listed in Table 8 below.

TABLE 8
Humanized antibody sequences (underlining indicates CDR; bold/italic indicates
back mutations)

| | | SEQ ID |
|---|---|---|
| 2p834 | Sequence | NO: |
| VHH | QSQLVESGGGLVQPGGSLRLSCAASGFAFRRYTMSWVRQAPGKGLEW | 2 |
| | VSTITHNGSITTYAESAQGRFTISRDNAKNTLYLQMNSLKPEDTAVY | |
| | YCAQGGGSNYYRESWGQGTQVTVSS | |
| VHH | EVOLVESGGGLVQPGGSLRLSCAASGFAFRRYTMSWVRQAPGKGLEW | 65 |
| (grafted) | VSTITHNGSITTYAESAQGRFTISRDNSKNTLYLQMNSLRAEDTAVY | |
| | YCAKGGGSNYYRESWGQGTQVTVSS | |
| VHHz2 | EVOLVESGGGLVQPGGSLRLSCAASGFAFRRYTMSWVRQAPGKGLEW | 66 |
| | VSTITHNGSITTYAESAQGRFTISRDNSKNTLYLQMNSLRAEDTAVY | |
| | YCAQGGGSNYYRESWGQGTQVTVSS | |
| VHHz3 | EVQLVESGGGLVQPGGSLRLSCAASGFAFRRYTMSWVRQAPGKGLEW | 67 |
| | VSTITHNGSITTYAESAQGRFTISRDNSKNTLYLQMNSLRPEDTAVY | |
| | YCAQGGGSNYYRESWGQGTQVTVSS | |
| VHHz4 | QSQLVESGGGLVQPGGSLRLSCAASGFAFRRYTMSWVRQAPGKGLEW | 68 |
| | VSTITHNGSITTYAESAQGRFTISRDNSKNTLYLQMNSLRPEDTAVY | |
| | YCAQGGGSNYYRESWGQGTQVTVSS | |

### 8.1 Cell-Line Based Functional Characterization of Claudin 18.2-CD40 Humanized Bispecific Antibodies

To assess the ability of humanized Claudin 18.2-CD40 bispecific antibodies to activate CD40 signaling pathway, a commercial CD40 NFκB luciferase reporter system was used, as described in Example 5.1. Briefly, H_CD40 (TNFRSF5) NFκB-Reporter Jurkat was used effector cells and CHO-K1-expressing or not expressing Claudin 18.2 as target cells. Effector cells were cocultured with target cells in a E/T Ratio of 1:1, in a white 96-well plate. Antibodies were 5-fold serially diluted and added to a white 96-well assay plate, at a final concentration ranging from 0.001 nM to 100 nM. Luminescence was obtained after 5 hours incubation by adding the substrate of luciferase and measured by a microplate reader.

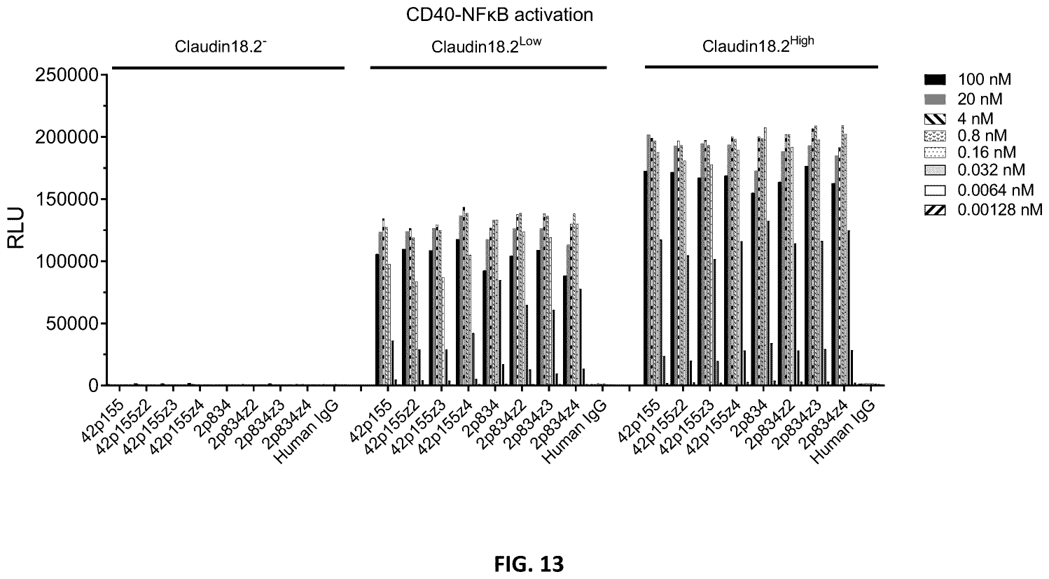

As shown in FIG. 13, humanized anti-CLDN18.2/CD40 bispecific antibodies showed comparable activity in inducing CD40 signaling pathway.

### 8.2 Activity of the Humanized Bispecific Antibodies to Promote Human Dendritic Cell Immune Response

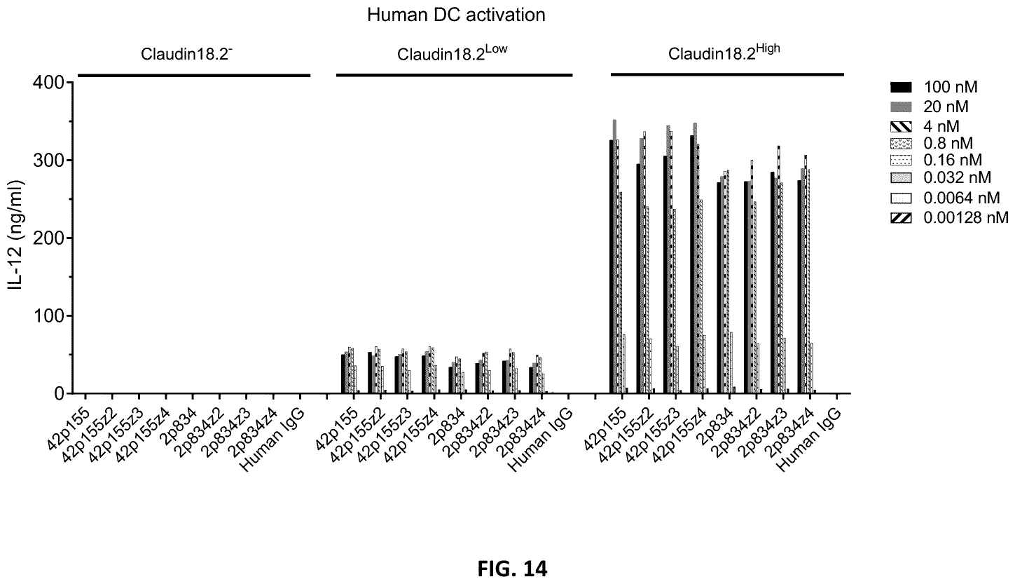

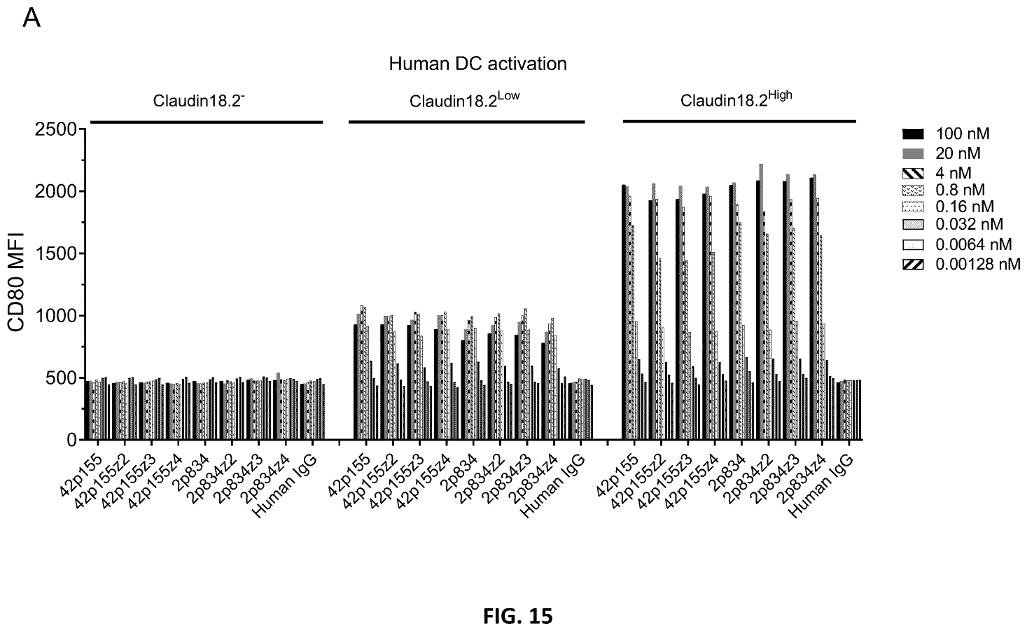

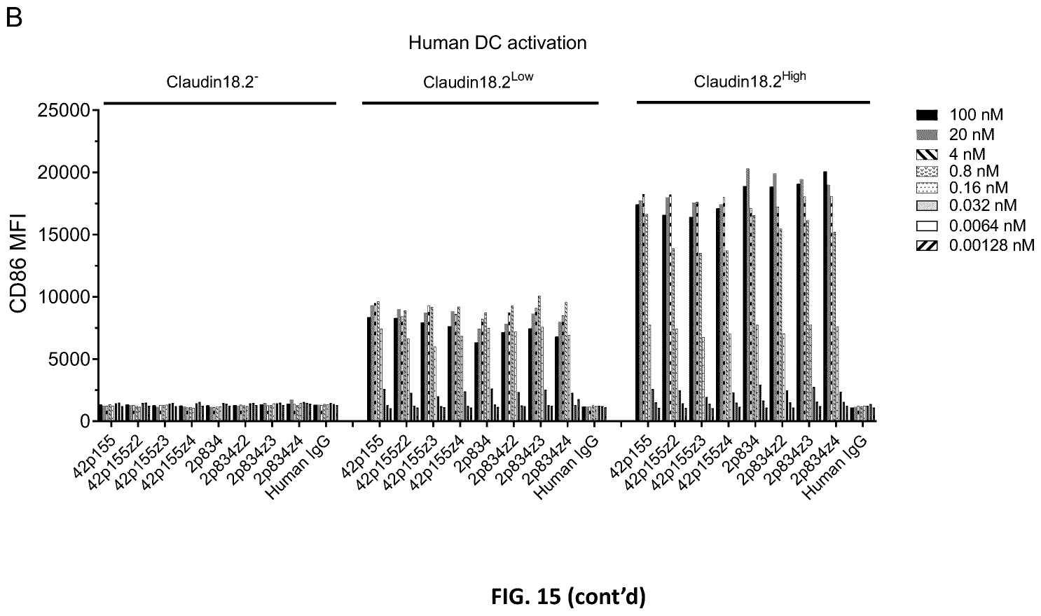

To investigate the ability of humanized CLDN18.2/CD40 bispecific antibodies to stimulate human dendritic cell (DC) response, IL-12 cytokine release and CD80/CD86 expression of DCs were examined, as described in the previous Example 5. Human DCs were obtained following the procedures specified in Example 3.1. Human DCs were used as the effector cells. CHO-K1 cells expressing Claudin 18.2 were used as the target cells. Human DCs (5×10⁴) were co-cultured with CHO-K1-Claudin 18.2 or parental CHO-K1 cells (1.5×10⁴) (E/T Ratio=3:1). Humanized bispecific antibodies were added to the culture medium at a final concentration starting from 100 nM. After 48 hours incubation, the level of IL-12 in the culture medium was measured using IL-12/p40) (human) LANCE Ultra TR-FRET Detection Kit (PerkinElmer). Moreover. CD80/CD86 expression of DCs were examined by FACS following the staining and analysis procedures specified in Example 5.2. As shown in FIGS. 14 and 15, the DC response (IL-12 secretion in FIG. 14 and CD80/CD86 expression in FIG. 15 A/B) induced by humanized bispecific antibodies were comparable with their chimeric antibodies.

### 8.3 Activity of the Humanized Bispecific Antibodies to Promote Human B Cell Immune Response

To investigate the ability of humanized Claudin 18.2-CD40 bispecific antibodies to activate human B cell. Ki67 (cell proliferation) and CD86 expression of B cells were examined. As described in Example 5, human B cells were isolated from human PBMC using B cell isolation kit according to the manufacturer's protocol. Human B cells were used as the effector cells. CHO-K1 cells expressing Claudin 18.2 were used as the target cells. Human B cells (5×10⁴) were co-cultured with CHO-K1-Claudin 18.2 or parental CHO-K1 cells (1×10⁴) (E/T Ratio=5:1). 5-fold serially diluted humanized bispecific antibodies were added to the culture medium at a final concentration starting from 100 nM. After 72 hours incubation. Ki67/CD86 expression of B cells were examined by FACS following the staining and analysis procedures specified in Example 5.3. As shown in FIG. 16, the B cell response (Ki67/CD86 expression in FIG. 16 A/B) induced by humanized bispecific antibodies were comparable with their chimeric antibodies.

### Example 9. Tumor Growth Inhibition by Anti-Claudin 18.2/CD40 Bispecific Antibodies

In this example, humanized mice that the extracellular domain of mouse CD40 were replaced with the counterpart human CD40, were used to test the anti-tumor activity of the bispecific antibodies.

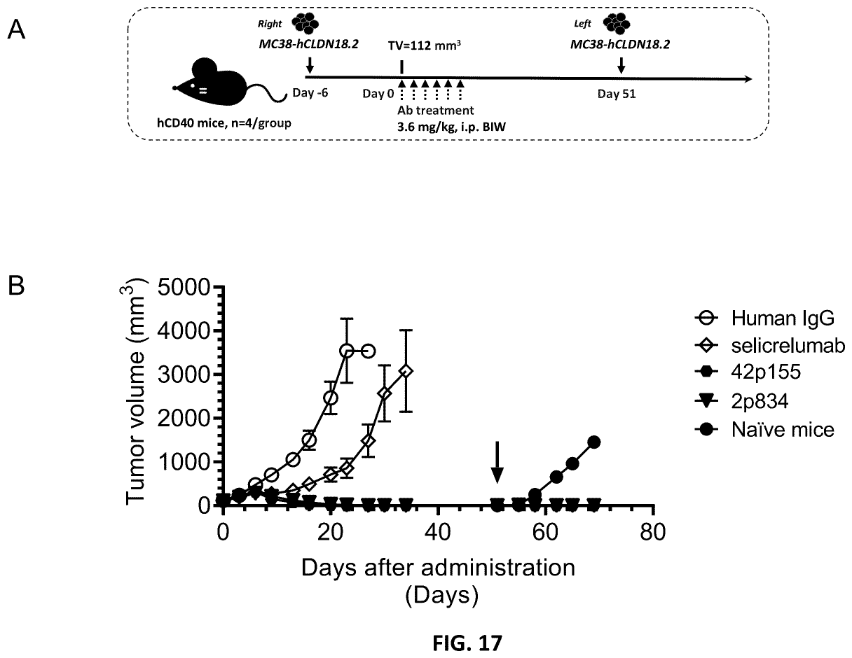

Mouse colon adenocarcinoma cells (MC38) were engineered to express human CLDN18.2. Humanized C57bl/6 mice (huCD40)) were subcutaneously implanted with MC38-hCLDN18.2 cells. As shown in FIG. 17A, mice were intraperitoneally administered twice a week for 6 times with following antibodies: human IgG control (3.6 mg/kg), selicrelumab (3 mg/kg), 42p155 anti-CLDN18.2/CD40 bispecific antibody (3.6 mg/kg, equal molar amount with selicrelumab), 2p834 anti-CLDN18.2/CD40 bispecific antibody (3.6 mg/kg, equal molar amount with selicrelumab).

As shown in FIG. 17B, all mice treated with 42p155 anti-CLDN18.2/CD40 biAb or 2p834 anti-CLDN18.2/CD40 biAb had complete tumor regression, while mice treated with selicrelumab only showed a moderated response. In addition, after 51 days upon the 1^{st} therapy, mice that were free of tumor were injected subcutaneously again on the opposite flank with MC38-hCLDN18.2 cells (indicated by the black arrow in FIG. 17B) in order to determine the formation of an immune cell memory against this tumor cell line. As a control, 4 naïve C57bl/6-huCD40 mice were injected with the same MC38-hCLDN18.2 tumor cells. Tumor volumes were monitored by caliper measurement twice per week for the duration of the experiment.

As shown in FIG. 17B and Table 9, selicrelumab can suppress the tumor growth with the TGI of 78.3% at D23 upon the 1st therapy. Meanwhile, both 42p155 and 2p834 bispecific antibodies induced a complete tumor remission in all treated mice since Day 27. In addition, upon re-challenge, no MC38-hCLDN18.2 tumor grew in the groups previously treated with 42p155 and 2p834 bispecific antibodies whereas in naïve mice 100% of the tumors grew. These results indicate a strong anti-tumor efficacy and tumor growth regression with the targeted-CD40 therapy in contrast to the non-targeted CD40 therapy. The re-challenge data indicate the formation of an efficient immune memory response against MC38-hCLDN18.2 tumor cells in all mice treated with 42p155 or 2p834 bispecific antibodies.

TABLE 9
TGI of MC38-CLDN18.2 mouse model.

| Group | TGI (%) at D 23 |
|---|---|
| selicrelumab | 78.3% |
| 42p155 BiAb | 103.2% |
| 2p834 BiAb | 102.8% |

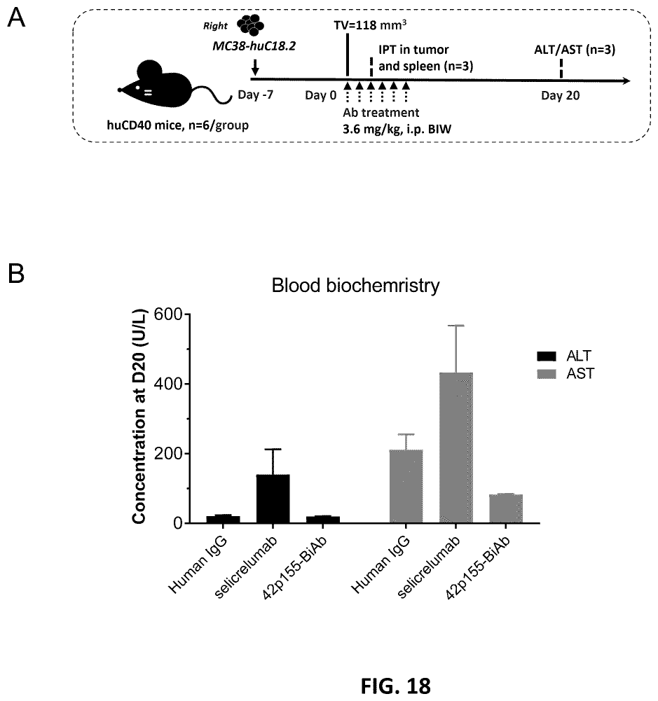

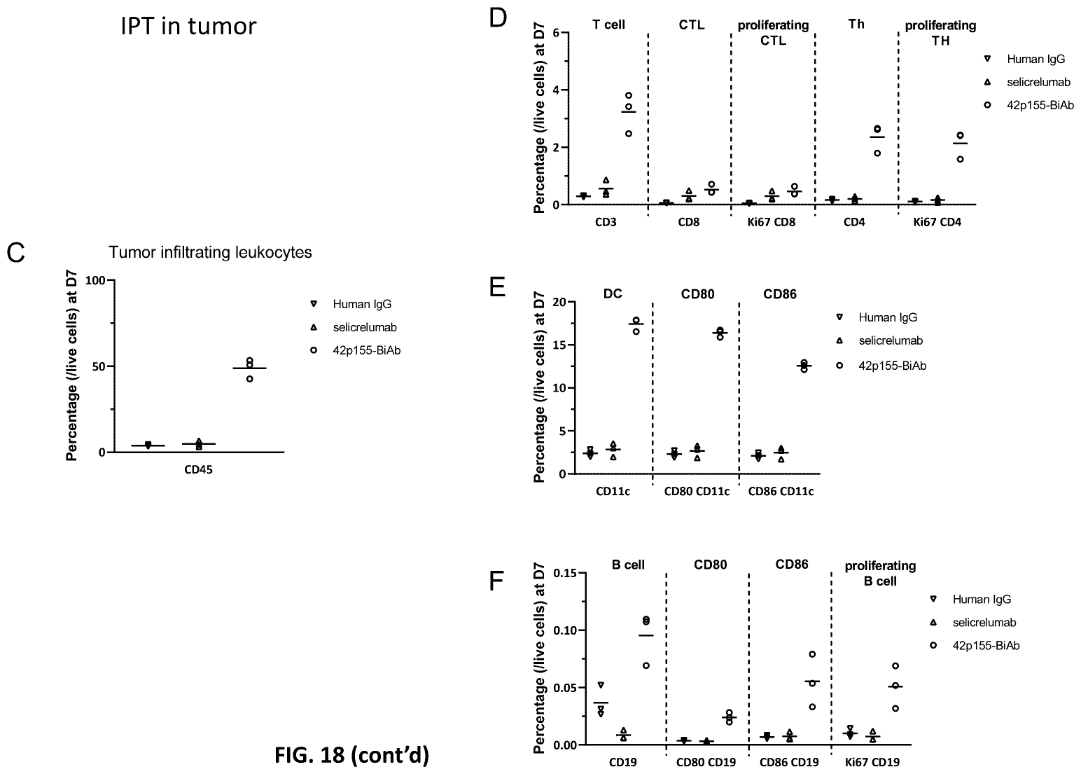

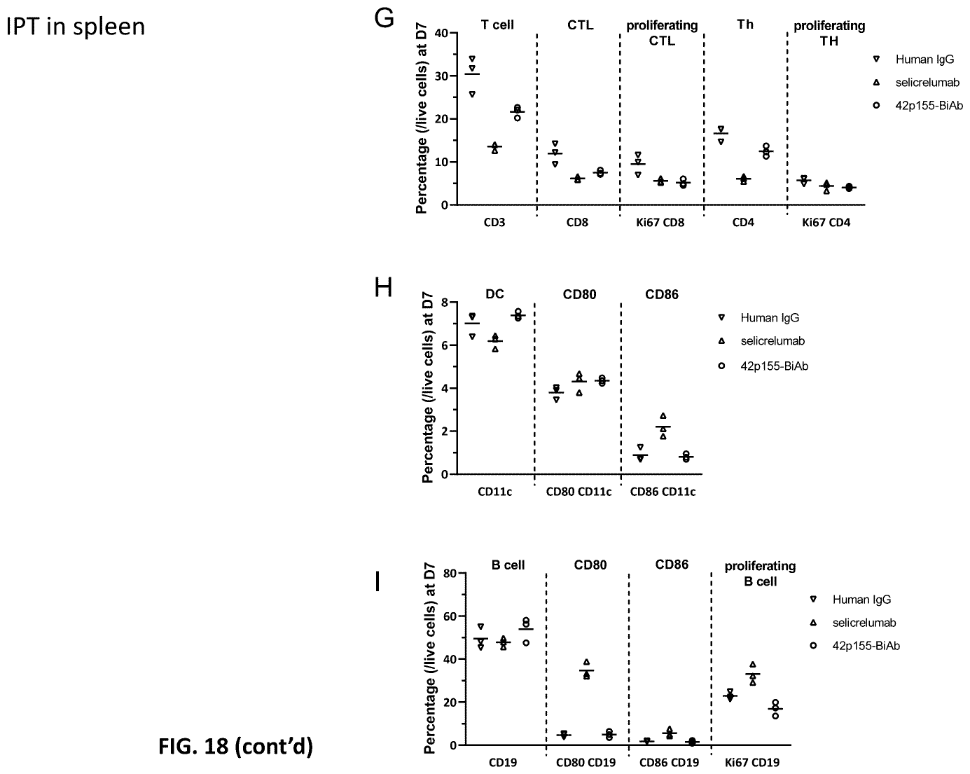

To assess the immune activation by CD40 therapy, tumor infiltrating immunophenotyping (IPT) analysis was performed at Day 7 following intraperitoneal administration twice a week for 2 times (FIG. 18A). As shown in FIG. 18C-F, 42p155 bispecific antibody evoked significant immune response in tumor tissue, including increase in percentage of leukocytes, T cells (both CD8⁺ and CD4⁺ T cells), DC and B cells, compared to both PBS and selicrelumab group. In contrast, selicrelumab only slightly increased percentage of T cells, specifically CD8⁺ T cells. Moreover, 42p155-BiAb also enhanced proliferation of CD8 T cells, CD4 T cells and B cells, as well as increased the expression of CD80 and CD86 in both DC and B cells, compared to both PBS and selicrelumab group. These data suggest that 42p155 bispecific antibody could boost significant immune response in tumor tissue, superior to CD40 agonistic monoclonal antibody.

To determine whether CD40 therapy had any effect on the peripheral immune system, immunophenotyping (IPT) analysis on the spleen was performed at Day 7 as well. As shown in FIG. 18G-I, 42p155-BiAb did not affect the peripheral immune cell population significantly, while selicrelumab decreased T cells. Meanwhile, activated B cells were increased as well upon treatment of selicrelumab. These data suggest that selicrelumab activated peripheral CD40 while 42p155-BiAb remained silent in the peripheral.

To evaluate the toxicity of CD40 activation on liver function, AST and ALT concentration in blood was detected at Day 20 following intraperitoneal administration twice a week for 6 times (FIG. 18A). As shown in FIG. 18B, 42p155 bispecific antibody did not increase either ALT or AST level compared with PBS group, while selicrelumab-treated group had increased both ALT and AST level. These data suggest 42p155 bispecific antibody could minimize the risk of peripheral toxicity via focused CD40 activation mainly in tumor environment.

### Example 10: Generation of Mouse Monoclonal Antibodies Against Human 5T4

This example describes the generation of anti-human 5T4 mouse monoclonal antibodies using the hybridoma technology.

Antigen: human 5T4-His protein and CHO-K1-expressing human 5T4 (CHOK1-hu5T4).

Immunization: To generate mouse monoclonal antibodies targeting human 5T4, SJL mice, Balb/c mice and C57BL/6 mice were first immunized with the 5T4-His protein. The immunized mice were subsequently boosted with the 5T4-His protein or CHO-K1-expressing human 5T4. To select mice producing antibodies that bound to 5T4 protein, the serum of immunized mice was subjected to the antibody titer evaluation by ELISA and FACS. Briefly, microtiter plates were coated with human 5T4 or cyno 5T4 protein at 0.5 or 1 μg/mL in ELISA coating buffer, 100 μL/well at 4° C. overnight, then blocked with 150 μL/well of 1% BSA. Dilutions of serum from immunized mice were added to each well and incubated for 1 hours at 37° C. The plates were washed with PBS/Tween and then incubate with anti-mouse IgG antibody conjugated with Horse Radish Peroxidase (HRP) for 30 min at 37° C. After washing, the plates were developed with TMB substrate and analyzed by spectrophotometer at OD 450 nm. Immune responses were also tested by serum FACS against CHOK1-hu5T4 cell line with CHOK1 parental cell line served as negative control. The resulting mice were used for fusions. The hybridoma supernatants were screened by ELISA.

Cell fusion: Fusion was performed by electro fusion. Fused cells were plated into 50 96-well plates for each fusion.

Screening: The hybridoma supernatants were screened by ELISA against recombinant human (rh) 5T4-His protein and recombinant cyno 5T4-His protein. Then, positive supernatants from primary screening underwent confirmative screening with FACS binding against CHOK1-hu5T4 cell line as well as protein binding with ELISA.

Subcloning and screening: Positive primary clones from each fusion were subcloned by limiting dilution to ensure that the subclones were derived from a single parental cell. Subcloning were screened in the same approach as primary clones and culture supernatant of positive clones underwent additional confirmative screening by affinity ranking.

The clones 14G12, 393E9, 159D5 and 286B4 were selected for further analysis and humanization. The test 5T4 mAbs were classified to four bins (bin A, bin B, bin C and bin D) based on the binding epitope on the human 5T4 by competitive ELISA. As a reference, the Fab portion of naptumomab binds to bin A of 5T4 as well. 14G12 and 393E9 belong to bin A, 159D5 belongs to bin B, and 286B4 belongs to bin D.

### Example 11. Humanization of 5T4 Antibodies

The variable region genes were employed to create humanized mAbs. In the first step of this process, the amino acid sequences of the VH and VK were compared against the available database of human Ig gene sequences to find the overall best-matching human germline Ig gene sequences.

The sequences of the human germlines used for CDR grafting, as well as the resulting humanized sequences are listed in Table 10.

TABLE 10-1
Humanization of 14G12 (underlining indicates CDR; bold/italic indicates back
mutations)

| | | SEQ ID |
|---|---|---|
| Name | Sequence | NO: |
| 14G12 VH | EVQLQQSGPELVKPGASVKISCKASGYAFSSSWMNWVKQRPGKGLEWIGR | 73 |
| | IYREDGHTNYNGKFKGKATLTADKSSTTAYMQLTSLTSEDSAVYFCANGG | |
| | AMDYWGQGTSVTVSS | |
| VH CDR1 | SSWMN | 75 |
| VH CDR2 | RIYREDGHTNYNGKFKG | 76 |
| VH CDR3 | GGAMDY | 77 |
| 14G12 hVH1 | QVQLVQSGAEVKKPGSSVKVSCKASGYAFSSSWMNWVRQAPGQGLEWMGR | 81 |
| | IYREDGHTNYNGKFKGRVTITADKSTTTAYMELSSLRSEDTAVYYCANGG | |
| | AMDYWGQGTLVTVSS | |
| hVH1 grafted | QVQLVQSGAEVKKPGSSVKVSCKASGGTFSSSWMNWVRQAPGQGLEWMGR | 82 |
| | IYREDGHTNYNGKFKGRVTITADKSTSTAYMELSSLRSEDTAVYYCANGG | |
| | AMDYWGQGTLVTVSS | |
| 14G12 hVH2 | QVQLVQSGAEVKKPGASVKVSCKASGYAFSSSWMNWVRQAPGQGLEWMGR | 83 |
| | IYREDGHTNYNGKFKGRVTMTADKSITTAYMELSRLRSDDTAVYYCANGG | |
| | AMDYWGQGTLVTVSS | |
| hVH2 grafted | QVQLVQSGAEVKKPGASVKVSCKASGYTFSSSWMNWVRQAPGQGLEWMGR | 84 |
| | IYREDGHTNYNGKFKGRVTMTADKSITTAYMELSRLRSDDTAVYYCANGG | |
| | AMDYWGQGTLVTVSS | |
| 14G12 hVH3 | QVQLVQSGAEVKKPGASVKVSCKASGYAFSSSWMNWVRQAPGQGLEWMGR | 85 |
| | IYREDGHTNYNGKFKGRVTMTADKSTTTAYMELSSLRSEDTAVYYCANGG | |
| | AMDYWGQGTLVTVSS | |
| hVH3 grafted | QVQLVQSGAEVKKPGASVKVSCKASGYTFSSSWMNWVRQAPGQGLEWMGR | 86 |
| | IYREDGHTNYNGKFKGRVTMTADKSTTTGYMELSSLRSEDTAVYYCANGG | |
| | AMDYWGQGTLVTVSS | |
| 14G12 hVH4 | QVQLVQSGAEVKKPGASVKVSCKASGYAFSSSWMNWVRQAPGQRLEWMGR | 87 |
| | IYREDGHTNYNGKFKGRVTITADKS | |
| | ATTAYMELSSLRSEDTAVYYCANGGAMDYWGQGTLVTVSS | |
| hVH4 grafted | QVQLVQSGAEVKKPGASVKVSCKASGYTFSSSWMNWVRQAPGQRLEWMGR | 88 |
| | IYREDGHTNYNGKFKGRVTITADKSATTAYMELSSLRSEDTAVYYCANGG | |
| | AMDYWGQGTLVTVSS | |
| 14G12 hVH5 | QVQLVQSGAEVKKPGASVKVSCKASGYAFSSSWMNWVRQAPGQGLEWMGR | 89 |
| | IYREDGHTNYNGKFKGRVTMTADKSTTTAYMELRSLRSDDTAVYYCANGG | |
| | AMDYWGQGTLVTVSS | |
| hVH5 grafted | QVQLVQSGAEVKKPGASVKVSCKASGYTFSSSWMNWVRQAPGQGLEWMGR | 90 |
| | IYREDGHTNYNGKFKGRVTMTADKSTSTAYMELRSLRSDDTAVYYCANGG | |
| | AMDYWGQGTLVTVSS | |
| 14G12 VL | DIQMTQSTSSLSASLGDRVTISCRASQDISNYLNWYQQKPDGTVKLLIYY | 74 |
| | TSRLHSGVPSRFSGSGSGTDYSLTISNLEQEDIATYFCQQDNTLPWTFGG | |
| | GTKLEIK | |
| VL CDR1 | RASQDISNYLN | 78 |
| VL CDR2 | YTSRLHS | 79 |
| VL CDR3 | QQDNTLPWT | 80 |
| 14G12 hVL1 | DIQMTQSPSSLSASVGDRVTITCRASQDISNYLNWYQQKPGKAPKLLIYY | 91 |
| | TSRLHSGVPSRFSGSGSGTDYTFTISSLQPEDIATYFCQQDNTLPWTFGG | |
| | GTKVEIK | |
| hVL1 grafted | DIQMTQSPSSLSASVGDRVTITCRASQDISNYLNWYQQKPGKAPKLLIYY | 92 |
| | TSRLHSGVPSRFSGSGSGTDYTFTISSLQPEDIATYYCQQDNTLPWTFGG | |
| | GTKVEIK | |
| 14G12 hVL2 | DIQMTQSPSSLSASVGDRVTITCRASQDISNYLNWYQQKPGKAPKLLIYY | 93 |
| | TSRLHSGVPSRFSGSGSGTDYTLTISSLQPEDIATYFCQQDNTLPWTFGG | |
| | GTKVEIK | |
| hVL2 grafted | DIQMTQSPSSLSASVGDRVTITCRASQDISNYLNWYQQKPGKAPKLLIYY | 94 |
| | TSRLHSGVPSRFSGSGSGTDYTLTISSLQPEDFATYFCQQDNTLPWTFGG | |
| | GTKVEIK | |
| 14G12 hVL3 | EIVMTQSPATLSLSPGERATLSCRASQDISNYLNWYQQKPGQAPRLLIYY | 95 |
| | TSRLHSGIPARFSGSGSGTDYTLTISSLEPEDIAVYFCQQDNTLPWTFGG | |
| | GTKVEIK | |
| hVL3 grafted | EIVMTQSPATLSLSPGERATLSCRASQDISNYLNWYQQKPGQAPRLLIYY | 96 |
| | TSRLHSGIPARFSGSGSGTDYTLTISSLEPEDFAVYYCQQDNTLPWTFGG | |
| | GTKVEIK | |
| 14G12 hVL4 | DIVMTQSPDSLAVSLGERATINCRASQDISNYLNWYQQKPGQPPKLLIYY | 97 |
| | TSRLHSGVPDRFSGSGSGTDYTLTISSLQAEDIAVYFCQQDNTLPWTFGG | |
| | GTKVEIK | |
| hVL4 grafted | DIVMTQSPDSLAVSLGERATINCRASQDISNYLNWYQQKPGQPPKLLIYY | 98 |
| | TSRLHSGVPDRFSGSGSGTDYTLTISSLQAEDIAVYYCQQDNTLPWTFGG | |
| | GTKVEIK | |
| 14G12 hVL5 | DIQMTQSPSTLSASVGDRVTITCRASQDISNYLNWYQQKPGKAPKLLIYY | 99 |
| | TSRLHSGVPSRFSGSGSGTEYTLTISSLQPDDIATYFCQQDNTLPWTFGG | |
| | GTKVEIK | |
| hVL5 grafted | DIQMTQSPSTLSASVGDRVTITCRASQDISNYLNWYQQKPGKAPKLLIYY | 100 |
| | TSRLHSGVPSRFSGSGSGTEYTLTISSLQPDDFATYFCQQDNTLPWTFGG | |
| | GTKVEIK | |

TABLE 10-2
Humanized antibodies from 14G12

| | VL | | | | |
|---|---|---|---|---|---|
| | 14G12- | 14G12- | 14G12- | 14G12- | 14G12- |
| VH | hVL1 | hVL2 | hVL3 | hVL4 | hVL5 |
| 14G12- | Hu14G12- | Hu14G12- | Hu14G12- | Hu14G12- | Hu14G12- |
| hVH1 | 23 | 24 | 25 | 26 | 27 |
| 14G12- | Hu14G12- | Hu14G12- | Hu14G12- | Hu14G12- | Hu14G12- |
| hVH2 | 28 | 29 | 30 | 31 | 32 |
| 14G12- | Hu14G12- | Hu14G12- | Hu14G12- | Hu14G12- | Hu14G12- |
| hVH3 | 33 | 34 | 35 | 36 | 37 |
| 14G12- | Hu14G12- | Hu14G12- | Hu14G12- | Hu14G12- | Hu14G12- |
| hVH4 | 38 | 39 | 40 | 41 | 42 |
| 14G12- | Hu14G12- | Hu14G12- | Hu14G12- | Hu14G12- | Hu14G12- |
| hVH5 | 43 | 44 | 45 | 46 | 47 |

TABLE 10-3
Humanization of 393E9 (underlining indicates CDR; bold/italic
indicates back mutations)

| | | SEQ |
|---|---|---|
| | | ID |
| Name | Sequence | NO: |
| 393E9_VH | EVKLVESGGGLVQPGGSLSLSCAASGFTFTDYYMSWVRQPPGKALEWLG | 101 |
| | FIRNKGNGYTTENSASVKGRFTISRDNSQSILYLQMNALRAEDSATYYC | |
| | ARYRGNPHYAMDYWGQGTSVTVSS | |
| VH CDR1 | TDYYMS | 103 |
| VH CDR2 | FIRNKGNGYTTENSASVKG | 104 |
| VH CDR3 | YRGNPHYAMDY | 105 |
| 393E9 VH- | EVKLVESGGGLVQPGGSLSLSCAASGFTFTDYYMSWVRQPPGKALEWLG | 109 |
| G57A | 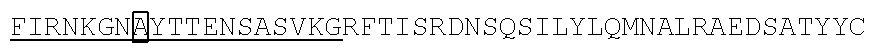 | |
| (CDR2 | ARYRGNPHYAMDYWGQGTSVTVSS | |
| mutated) | | |
| 393E9 | EVQLVESGGGLVQPGGSLRLSCAASGFTFTDYYMSWVRQAPGKGLEWVG | 110 |
| VH.V1 | FIRNKGNGYTTENSASVKGRFTISRDNSKSTLYLQMNSLRAEDTAVYYC | |
| | ARYRGNPHYAMDYWGQGTLVTVSS | |
| 393E9 | EVQLVESGGGLIQPGGSLRLSCAASGFTFTDYYMSWVRQPPGKGLEWVG | 111 |
| VH.V2 | FIRNKGNGYTTENSASVKGRFTISRDNSKSTLYLQMNSLRAEDTAVYYC | |
| | ARYRGNPHYAMDYWGQGTLVTVSS | |
| 393E9 | QVQLVESGGGVVQPGGSLRLSCAASGFTFTDYYMSWVRQAPGKGLEWVG | 112 |
| VH.V3 | FIRNKGNGYTTENSASVKGRFTISRDNSKSTLYLQMNSLRAEDTAVYYC | |
| | ARYRGNPHYAMDYWGQGTLVTVSS | |
| 393E9 | EVQLVESGGGLVQPGGSLRLSCAASGFTFTDYYMSWVRQAPGKGLEWVG | 113 |
| VH.V4 | FIRNKGNGYTTENSASVKGRFTISRDNSKSSLYLQMNSLKTEDTAVYYC | |
| | ARYRGNPHYAMDYWGQGTLVTVSS | |
| 393E9 | EVQLVESGGGLVQPGRSLRLSCTASGFTETDYYMSWVRQAPGKGLEWVG | 114 |
| VH.V5 | FIRNKGNGYTTENSASVKGRFTISRDNSKSILYLQMNSLKTEDTAVYYC | |
| | ARYRGNPHYAMDYWGQGTLVTVSS | |
| 393E9 | EVQLVESGGGLVQPGGSLRLSCAASGFTFTDYYMSWVRQAPGKGLEWVG | 115 |
| VH.V6 | FIRNKGNGYTTENSASVKGRFTISRDNAKSSLYLQMNSLRAEDTAVYYC | |
| | ARYRGNPHYAMDYWGQGTLVTVSS | |
| 393E9_VL | DIVLTQFPASLAVSLGQRATISCRASESVEFYGTSFLQWYQQKPGQPPK | 102 |
| | LLIYGASNVESGVPARFSGSGSGTDESLSIHPVEEDDIAMYFCQQSRKV | |
| | PSTFGGGTKLEIK | |
| VL CDR1 | RASESVEFYGTSFLQ | 106 |
| VL CDR2 | GASNVES | 107 |
| VL CDR3 | QQSRKVPST | 108 |
| 393E9 | DIVLTQSPDSLAVSLGERATINCRASESVEFYGTSFLQWYQQKPGQPPK | 116 |
| VL.V1 | LLIYGASNVESGVPDRFSGSGSGTDETLTISSLQAEDIAVYFCQQSRKV | |
| | PSTFGGGTKVEIK | |
| 393E9 | EIVLTQSPATLSVSPGERATLSCRASESVEFYGTSFLQWYQQKPGQAPR | 117 |
| VL.V2 | LLIYGASNVESGIPARFSGSGSGTEFTLTISSLQSEDIAVYFCQQSRKV | |
| | PSTFGGGTKVEIK | |
| 393E9 | DIQLTQSPSSLSASVGDRVTITCRASESVEFYGTSFLQWYQQKPGKAPK | 118 |
| VL.V3 | LLIYGASNVESGVPSRESGSGSGTDFTLTISSLQPEDIATYFCQQSRKV | |
| | PSTFGGGTKVEIK | |
| 393E9 | DIVLTQSPLSLPVTPGEPASISCRASESVEFYGTSFLQWYLQKPGQSPQ | 119 |
| VL.V4 | LLIYGASNVESGVPDRESGSGSGTDFTLKISRVEAEDIGVYFCQQSRKV | |
| | PSTFGGGTKVEIK | |
| 393E9 | EIVLTQSPGTLSLSPGERATLSCRASESVEFYGTSFLQWYQQKPGQAPR | 120 |
| VL.V5 | LLIYGASNVESGIPDRESGSGSGTDFTLTISRLEPEDIAVYFCQQSRKV | |
| | PSTFGGGTKVEIK | |

TABLE 10-4
Humanized antibodies from 393E9

| | 393E9 VH.V1 | 393E9 VH.V2 | 393E9 VH.V3 | 393E9 VH.V4 | 393E9 VH.V5 | 393E9 VH.V6 |
|---|---|---|---|---|---|---|
| 393E9 VL.V1 | 393E9_hVH1_{—} | 393E9_hVH2_{—} | 393E9_hVH3_{—} | 393E9_hVH4_{—} | 393E9_hVH5_{—} | 393E9_hVH6_{—} |
| | hVL1 | hVL1 | hVL1 | hVL1 | hVL1 | hVL1 |
| 393E9 VL.V2 | 393E9_hVH1_{—} | 393E9_hVH2_{—} | 393E9_hVH3_{—} | 393E9_hVH4_{—} | 393E9_hVH5_{—} | 393E9_hVH6_{—} |
| | hVL2 | hVL2 | hVL2 | hVL2 | hVL2 | hVL2 |
| 393E9 VL.V3 | 393E9_hVH1_{—} | 393E9_hVH2_{—} | 393E9_hVH3_{—} | 393E9_hVH4_{—} | 393E9_hVH5_{—} | 393E9_hVH6_{—} |
| | hVL3 | hVL3 | hVL3 | hVL3 | hVL3 | hVL3 |
| 393E9 VL.V4 | 393E9_hVH1_{—} | 393E9_hVH2_{—} | 393E9_hVH3_{—} | 393E9_hVH4_{—} | 393E9_hVH5_{—} | 393E9_hVH6_{—} |
| | hVL4 | hVL4 | hVL4 | hVL4 | hVL4 | hVL4 |
| 393E9 VL.V5 | 393E9_hVH1_{—} | 393E9_hVH2_{—} | 393E9_hVH3_{—} | 393E9_hVH4_{—} | 393E9_hVH5_{—} | 393E9_hVH6_{—} |
| | hVL5 | hVL5 | hVL5 | hVL5 | hVL5 | hVL5 |

TABLE 10-5
Humanization of 159D5 (underlining indicates CDR; bold/italic indicates
back mutations)

| | | SEQ ID |
|---|---|---|
| Name | Sequence | NO: |
| 159D5 VH | DVQLVESGGGLVQPGGSRKLSCTASGFTFSNFGMHWVRQAPETGLEWVAY | 121 |
| | ISSGSSTFYYADSVKGRFTISRDNPKNTLFLQMTSLRSEDTAMYFCARSP | |
| | AYYRYGLDYWGQGTTLTVSS | |
| VH CDR1 | NFGMH | 123 |
| VH CDR2 | YISSGSSTFYYADSVKG | 124 |
| VH CDR3 | SPAYYRYGLDY | 125 |
| 159D5 mVH- | DVQLVESGGGLVQPGGSRKLSCTASGFTFSNFGMHWVRQAPETGLEWVAY | 129 |
| S62A (CDR2 | 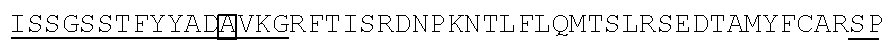 | |
| mutated) | AYYRYGLDYWGQGTTLTVSS | |
| 159D5 VH.V0 | EVQLVESGGGVVQPGRSLRLSCAASGFTFSNFGMHWVRQAPGKGLEWVAY | 130 |
| (grafted) | ISSGSSTFYYADSVKGRFTISRDNSKNTLYLQMNSLRAEDTAVYYCARSP | |
| | AYYRYGLDYWGQGTTVTVSS | |
| 159D5 VH.V1 | EVQLVESGGGVVQPGRSLRLSCAASGFTFSNFGMHWVRQAPGKGLEWVAY | 131 |
| | ISSGSSTFYYADSVKGRFTISRDNSKNTLYLQMNSLRAEDTAVYFCARSP | |
| | AYYRYGLDYWGQGTTVTVSS | |
| 159D5 VL | DIVLTQSPASLAVSLGQRATISCRASQSVSSSTFSYMHWYQQKPGQPPKL | 122 |
| | LIKSSSNLESGVPARFSGSGSGTDFTLNIHPVEEEDTATYYCQHSWEIPY | |
| | TFGGGTKLEIK | |
| VL CDR1 | RASQSVSSSTFSYMH | 126 |
| VL CDR2 | SSSNLES | 127 |
| VL CDR3 | QHSWEIPYT | 128 |
| 159D5 VL.V0 | DIVMTQTPLSLSVTPGQPASISCRASQSVSSSTFSYMHWYQQKPGQPPQL | 132 |
| (grafted) | LIYSSSNLESGVPDRFSGSGSGTDFTLKISRVEAEDVGVYYCQHSWEIPY | |
| | TFGGGTKVEIK | |
| 159D5 VL.V1 | DIVMTQTPLSLSVTPGQPASISCRASQSVSSSTFSYMHWYQQKPGQPPQL | 133 |
| | LIKSSSNLESGVPDRESGSGSGTDFTLKISRVEAEDVGVYYCQHSWEIPY | |
| | TFGGGTKVEIK | |
| 159D5 VL.V2 | DIVLTQTPLSLSVTPGQPASISCRASQSVSSSTESYMHWYQQKPGQPPQL | 134 |
| | LIKSSSNLESGVPDRESGSGSGTDFTLKISRVEAEDVGVYYCQHSWEIPY | |
| | TFGGGTKVEIK | |
| 159D5 VL.V3 | DIVMTQSPDSLAVSLGERATINCRASQSVSSSTFSYMHWYQQKPGQPPKL | 135 |
| (grafted) | LIYSSSNLESGVPDRESGSGSGTDFTLTISSLQAEDVAVYYCQHSWEIPY | |
| | TFGGGTKVEIK | |
| 159D5 VL.V4 | DIVMTQSPDSLAVSLGERATINCRASQSVSSSTFSYMHWYQQKPGQPPKL | 136 |
| | LIKSSSNLESGVPDRESGSGSGTDFTLTISSLQAEDVAVYYCQHSWEIPY | |
| | TFGGGTKVEIK | |
| 159D5 VL.V5 | DIVLTQSPDSLAVSLGERATINCRASQSVSSSTESYMHWYQQKPGQPPKL | 137 |
| | LIKSSSNLESGVPDRFSGSGSGTDFTLTISSLQAEDVAVYYCQHSWEIPY | |
| | TFGGGTKVEIK | |

TABLE 10-6
Humanized antibodies from 159D5

| | VL | | | | | | |
|---|---|---|---|---|---|---|---|
| VH | 159D5VL.V0 | 159D5VL.V1 | 159D5VL.V2 | 159D5VL.V3 | 159D5VL.V4 | 159D5VL.V5 | 159D5 VL |
| 159D5- | Hu159D5- | Hu159D5- | Hu159D5- | Hu159D5- | Hu159D5- | Hu159D5- | |
| VH.V0 | 1 | 2 | 3 | 4 | 5 | 6 | |
| 159D5- | Hu159D5- | Hu159D5- | Hu159D5- | Hu159D5- | Hu159D5- | Hu159D5- | |
| VH.V1 | 7 | 8 | 9 | 10 | 11 | 12 | |
| 159D5- | | | | | | | 159D5-P1 |
| VH.S62A | | | | | | | |

TABLE 10-7
Humanization of 286B4 (underlining indicates CDR; bold/italic indicates back
mutations, boxed indicates potential PTM site removal)

| | | SEQ ID |
|---|---|---|
| Name | Sequence | NO: |
| 286B4 VH | DVQLHESGPGLVKPSQSLSLTCSVTGYSITNNYYWNWIRQFPGNNLEWMGYI | 138 |
| | TYDGSNNYNPSLKNRISVTRDTSKNQFFLKLSSVTTEDTATYYCTRGGGQLR | |
| | FDYWGQGTTLTVSS | |
| VH CDR1 | NNYYWN | 140 |
| VH CDR2 | YITYDGSNNYNPSLKN | 141 |
| VH CDR3 | GGGQLRFDY | 142 |
| 286B4 VH- | DVQLHESGPGLVKPSQSLSLTCSVTGYSITNNYYWNWIRQFPGNNLEWMGYI | 146 |
| G55A | 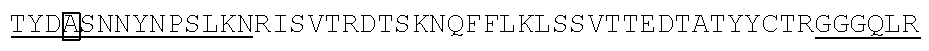 | |
| (CDR2 | FDYWGQGTTLTVSS | |
| mutated) | | |
| 286B4 | EVQLQESGPGLVKPSDTLSLTCAVSGYSISNNYYWNWIRQPPGKGLEWIGYI | 147 |
| VH.V0 | TYDGSNNYNPSLKNRVTMSVDTSKNQFSLKLSSVTAVDTAVYYCARGGGQLR | |
| (grafted) | FDYWGQGTTVTVSS | |
| 286B4 | EVQLQESGPGLVKPSDTLSLTCAVSGYSISNNYYWNWIRQPPGKGLEWIGYI | 148 |
| VH.V1 | TYDGSNNYNPSLKNRVTMSRDTSKNQFSLKLSSVTAVDTAVYYCARGGGQLR | |
| | FDYWGQGTTVTVSS | |
| 286B4 | EVQLQESGPGLVKPSDTLSLTCAVSGYSISNNYYWNWIRQPPGKGLEWIGYI | 149 |
| VH.V2 | TYDGSNNYNPSLKNRITMSRDTSKNQFSLKLSSVTAVDTAVYYCARGGGQLR | |
| | FDYWGQGTTVTVSS | |
| 286B4 | EVQLQESGPGLVKPSDTLSLTCAVSGYSISNNYYWNWIRQPPGKGLEWMGYI | 150 |
| VH.V3 | TYDGSNNYNPSLKNRITVSRDTSKNQFSLKLSSVTAVDTAVYYCARGGGQLR | |
| | FDYWGQGTTVTVSS | |
| 286B4 | EVQLQESGPGLVKPSDTLSLTCAVSGYSITNNYYWNWIRQPPGKGLEWMGYI | 151 |
| VH.V4 | TYDGSNNYNPSLKNRITVSRDTSKNQFSLKLSSVTAVDTAVYYCTRGGGQLR | |
| | FDYWGQGTTVTVSS | |
| 286B4 VL | SIVMTQTPKFLIVSAGDRVTITCKASQSVSNEVTWYQQKPGQSPKMLIYYAS | 139 |
| | NRYTGVPDRFTGSGYGTDFTFTINTVQAEDLAIYFCQQDYSSPWTFGGGTKL | |
| | EIK | |
| VL CDR1 | KASQSVSNEVT | 143 |
| VL CDR2 | YASNRYT | 144 |
| VL CDR3 | QQDYSSPWT | 145 |
| 286B4 | DIQMTQSPSSLSASVGDRVTITCKASQSVSNEVTWYQQKPGKAPKLLIYYAS | 152 |
| VL.V0 | NRYTGVPSRFSGSGSGTDFTFTISSLQPEDIATYYCQQDYSSPWTFGGGTKV | |
| (grafted) | EIK | |
| 286B4 | DIQMTQSPSSLSASVGDRVTITCKASQSVSNEVTWYQQKPGKAPKLLIYYAS | 153 |
| VL.V1 | NRYTGVPDRESGSGSGTDFTFTISSLQPEDIATYYCQQDYSSPWTFGGGTKV | |
| | EIK | |
| 286B4 | DIQMTQSPSSLSASVGDRVTITCKASQSVSNEVTWYQQKPGKAPKMLIYYAS | 154 |
| VL.V2 | NRYTGVPDRESGSGSGTDETFTISSLQPEDIATYYCQQDYSSPWTFGGGTKV | |
| | EIK | |
| 286B4 | DIQMTQSPSSLSASVGDRVTITCKASQSVSNEVTWYQQKPGKAPKMLIYYAS | 155 |
| VL.V3 | NRYTGVPDRESGSGYGTDETFTISSLQPEDIATYYCQQDYSSPWTFGGGTKV | |
| | EIK | |
| 286B4 | DIQMTQSPSSLSASVGDRVTITCKASQSVSNEVTWYQQKPGKSPKMLIYYAS | 156 |
| VL. V4 | NRYTGVPDRFSGSGYGTDFTFTISSLQPEDIATYFCQQDYSSPWTFGGGTKV | |
| | EIK | |
| 286B4 | SIVMTQSPSSLSASVGDRVTITCKASQSVSNEVTWYQQKPGKSPKMLIYYAS | 157 |
| VL.V5 | NRYTGVPDRESGSGYGTDFTFTISSLQPEDIATYFCQQDYSSPWTFGGGTKV | |
| | EIK | |

TABLE 10-8
Humanized antibodies from 286B4

| | VL | | | | | |
|---|---|---|---|---|---|---|
| VH | 286B4VL.V1 | 286B4VL.V2 | 286B4VL.V3 | 286B4VL.V4 | 286B4VL.V5 | 286B4 VL |
| 286B4-VH.V1 | Hu286B4-1 | Hu286B4-2 | Hu286B4-3 | Hu286B4-4 | Hu286B4-5 | |
| 286B4-VH.V2 | Hu286B4-6 | Hu286B4-7 | Hu286B4-8 | Hu286B4-9 | Hu286B4-10 | |
| 286B4-VH.V3 | Hu286B4-11 | Hu286B4-12 | Hu286B4-13 | Hu286B4-14 | Hu286B4-15 | |
| 286B4-VH.V4 | Hu286B4-16 | Hu286B4-17 | Hu286B4-18 | Hu286B4-19 | Hu286B4-20 | |
| 286B4-VH.G55A | | | | | | 286B4-P1 |

### Example 12. Selection of the Anti-5T4 Portion for 5T4-CD40 Bispecific Antibodies

This example tested the functional activities of 5T4-CD40 bispecific antibodies and selected the 5T4 portion for 5T4-CD40 bispecific antibodies.

Generation of 5T4-CD40 Bispecific Antibodies with Different 5T4 Binding Epitopes

The 5T4 sequences of 14G12, 393E9, 159D5 and 286B4 were constructed into 5T4 part of “2+2 b11” format, as shown in FIG. 4. 14G12 and 393E9 belong to bin A, 159D5 belongs to bin B, while 286B4 belongs to bin D. The CD40 sequence of 42p155 was used as the anti-CD40 part. The peptide chains of the “2+2 b11” format bispecific antibodies are shown in Table. 11A-D.

TABLE 11A
Peptide Chains of 14G12-42p155 Bispecific Antibodies

| | | | SEQ ID |
|---|---|---|---|
| | Name | Sequence | NO: |
| Heavy chain | 5T4 VH | EVQLQQSGPELVKPGASVKISCKASGYAFSSSWMNWVKQRP | 73 |
| | | GKGLEWIGRIYREDGHTNYNGKFKGKATLTADKSSTTAYMQ | |
| | | LTSLTSEDSAVYFCANGGAMDYWGQGTSVTVSS | |
| | CH (Human | ASTKGPSVFPLAPSSKSTSGGTAALGCLVKDYFPEPVTVSW | 158 |
| | IgG1 | NSGALTSGVHTFPAVLQSSGLYSLSSVVTVPSSSLGTQTYI | |
| | N297A) | CNVNHKPSNTKVDKKVEPKSCDKTHTCPPCPAPELLGGPSV | |
| | | FLFPPKPKDTLMISRTPEVTCVVVDVSHEDPEVKFNWYVDG | |
| | | VEVHNAKTKPREEQYASTYRVVSVLTVLHQDWLNGKEYKCK | |
| | | VSNKALPAPIEKTISKAKGQPREPQVYTLPPSREEMTKNQV | |
| | | SLTCLVKGFYPSDIAVEWESNGQPENNYKTTPPVLDSDGSF | |
| | | FLYSKLTVDKSRWQQGNVESCSVMHEALHNHYTQKSLSLSP | |
| | | GK | |
| | Linker | GGGGSGGGGSGGGGSGGGGS | 159 |
| | CD40 VHH | PSQLVESGGGLVQPGGSLRLSCAASGFAFRSYTMSWVRQAP | 1 |
| | | GKGLEWVSTITHNGAITTYAESAQGRFTISRDNAKNTLYLQ | |
| | | MNSLKPEDTAVYYCAQGGGSNYYRESWGQGTQVTVSS | |
| Light chain | 5T4 VL | DIQMTQSTSSLSASLGDRVTISCRASQDISNYLNWYQQKPD | 74 |
| | | GTVKLLIYYTSRLHSGVPSRFSGSGSGTDYSLTISNLEQED | |
| | | IATYFCQQDNTLPWTFGGGTKLEIK | |
| | CL (human | RTVAAPSVFIFPPSDEQLKSGTASVVCLLNNFYPREAKVQW | 160 |
| | kappa) | KVDNALQSGNSQESVTEQDSKDSTYSLSSTLTLSKADYEKH | |
| | | KVYACEVTHQGLSSPVTKSFNRGEC | |

TABLE 11B
Peptide Chains of 393E9-42p155 Bispecific Antibodies

| | | | SEQ ID |
|---|---|---|---|
| | Name | Sequence | NO: |
| Heavy chain | 5T4 VH | EVKLVESGGGLVQPGGSLSLSCAASGFTFTDYYMSWVRQP | 101 |
| | | PGKALEWLGFIRNKGNGYTTENSASVKGRFTISRDNSQSI | |
| | | LYLQMNALRAEDSATYYCARYRGNPHYAMDYWGQGTSVTV | |
| | | SS | |
| | CH (Human | ASTKGPSVFPLAPSSKSTSGGTAALGCLVKDYFPEPVTVS | 158 |
| | IgG1 | WNSGALTSGVHTFPAVLQSSGLYSLSSVVTVPSSSLGTQT | |
| | N297A) | YICNVNHKPSNTKVDKKVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYASTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | Linker | GGGGGGGGSGGGGSGGGGS | 159 |
| | CD40 VHH | PSQLVESGGGLVQPGGSLRLSCAASGFAFRSYTMSWVRQA | 1 |
| | | PGKGLEWVSTITHNGAITTYAESAQGRFTISRDNAKNTLY | |
| | | LQMNSLKPEDTAVYYCAQGGGSNYYRESWGQGTQVTVSS | |
| Light chain | 5T4 VL | DIVLTQFPASLAVSLGQRATISCRASESVEFYGTSFLQWY | 102 |
| | | QQKPGQPPKLLIYGASNVESGVPARFSGSGSGTDESLSIH | |
| | | PVEEDDIAMYFCQQSRKVPSTFGGGTKLEIK | |
| | CL (human | RTVAAPSVFIFPPSDEQLKSGTASVVCLLNNFYPREAKVQ | 160 |
| | kappa) | WKVDNALQSGNSQESVTEQDSKDSTYSLSSTLTLSKADYE | |
| | | KHKVYACEVTHQGLSSPVTKSFNRGEC | |

TABLE 11C
Peptide Chains of 159D5-42p155 Bispecific Antibodies

| | | | SEQ ID |
|---|---|---|---|
| | Name | Sequence | NO: |
| Heavy chain | 5T4 VH | DVQLVESGGGLVQPGGSRKLSCTASGFTFSNFGMHWVRQA | 121 |
| | | PETGLEWVAYISSGSSTFYYADSVKGRFTISRDNPKNTLF | |
| | | LQMTSLRSEDTAMYFCARSPAYYRYGLDYWGQGTTLTVSS | |
| | CH (Human | ASTKGPSVFPLAPSSKSTSGGTAALGCLVKDYFPEPVTVS | 158 |
| | IgG1 | WNSGALTSGVHTFPAVLQSSGLYSLSSVVTVPSSSLGTQT | |
| | N297A) | YICNVNHKPSNTKVDKKVEPKSCDKTHTCPPCPAPELLGG | |
| | | PSVELFPPKPKDTLMISRTPEVTCVVVDVSHEDPEVKFNW | |
| | | YVDGVEVHNAKTKPREEQYASTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | Linker | GGGGSGGGGSGGGGSGGGGS | 159 |
| | CD40 VHH | PSQLVESGGGLVQPGGSLRLSCAASGFAFRSYTMSWVRQA | 1 |
| | | PGKGLEWVSTITHNGAITTYAESAQGRFTISRDNAKNTLY | |
| | | LQMNSLKPEDTAVYYCAQGGGSNYYRESWGQGTQVTVSS | |
| Light chain | 5T4 VL | DIVLTQSPASLAVSLGQRATISCRASQSVSSSTFSYMHWY | 122 |
| | | QQKPGQPPKLLIKSSSNLESGVPARFSGSGSGTDFTLNIH | |
| | | PVEEEDTATYYCQHSWEIPYTFGGGTKLEIK | |
| | CL (human | RTVAAPSVFIFPPSDEQLKSGTASVVCLLNNFYPREAKVQ | 160 |
| | kappa) | WKVDNALQSGNSQESVTEQDSKDSTYSLSSTLTLSKADYE | |
| | | KHKVYACEVTHQGLSSPVTKSFNRGEC | |

TABLE 11D
Peptide Chains of 286B4-42p155 Bispecific Antibodies

| | | | SEQ ID |
|---|---|---|---|
| | Name | Sequence | NO: |
| Heavy chain | 5T4 VH | DVQLHESGPGLVKPSQSLSLTCSVTGYSITNNYYWNWIRQ | 138 |
| | | FPGNNLEWMGYITYDGSNNYNPSLKNRISVTRDTSKNQFF | |
| | | LKLSSVTTEDTATYYCTRGGGQLRFDYWGQGTTLTVSS | |
| | CH (Human | ASTKGPSVFPLAPSSKSTSGGTAALGCLVKDYFPEPVTVS | 158 |
| | IgG1 | WNSGALTSGVHTFPAVLQSSGLYSLSSVVTVPSSSLGTQT | |
| | N297A) | YICNVNHKPSNTKVDKKVEPKSCDKTHTCPPCPAPELLGG | |
| | | PSVFLFPPKPKDTLMISRTPEVTCVVVDVSHEDPEVKFNW | |
| | | YVDGVEVHNAKTKPREEQYASTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPGK | |
| | Linker | GGGGSGGGGSGGGGSGGGGS | 159 |
| | CD40 VHH | PSQLVESGGGLVQPGGSLRLSCAASGFAFRSYTMSWVRQA | 1 |
| | | PGKGLEWVSTITHNGAITTYAESAQGRFTISRDNAKNTLY | |
| | | LQMNSLKPEDTAVYYCAQGGGSNYYRESWGQGTQVTVSS | |
| Light chain | 5T4 VL | SIVMTQTPKFLIVSAGDRVTITCKASQSVSNEVTWYQQKP | 139 |
| | | GQSPKMLIYYASNRYTGVPDRFTGSGYGTDFTFTINTVQA | |
| | | EDLAIYFCQQDYSSPWTFGGGTKLEIK | |
| | CL (human | RTVAAPSVFIFPPSDEQLKSGTASVVCLLNNFYPREAKVQ | 160 |
| | kappa) | WKVDNALQSGNSQESVTEQDSKDSTYSLSSTLTLSKADYE | |
| | | KHKVYACEVTHQGLSSPVTKSFNRGEC | |

Cell-Line Based Functional Characterization of 5T4-CD40 Bispecific Antibodies with Different 5T4 Binding Epitopes

To assess the ability of the 5T4-CD40 bispecific antibodies to activate CD40 signaling pathway, a commercial CD40 NF-κB luciferase reporter system was used. In this assay, H_CD40 (TNFRSF5) NFκB-Reporter Jurkat (Genomeditech, cat #GM-C09520) was used as effector cells, and cells expressing human 5T4 (MCF-7; 5T4^{Low} and CHO-K1-hu5T4; 5′T4^{High}) or not (CHO-K1) were used as target cells. Briefly, effector cells at a density of 2.0×10⁴ cells per well were cocultured with 2.0×10⁴ target cells (E/T Ratio=1:1) in a white 96-well plate. Antibodies were 10-fold serially diluted and added to a white 96-well assay plate, at a final concentration ranging from 0.001 nM to 100 nM. After 5 hours incubation at 37° C., luminescence was obtained by adding the substrate of luciferase and measured by a microplate reader. Four-parameter logistic curve analysis was performed with GraphPad software.

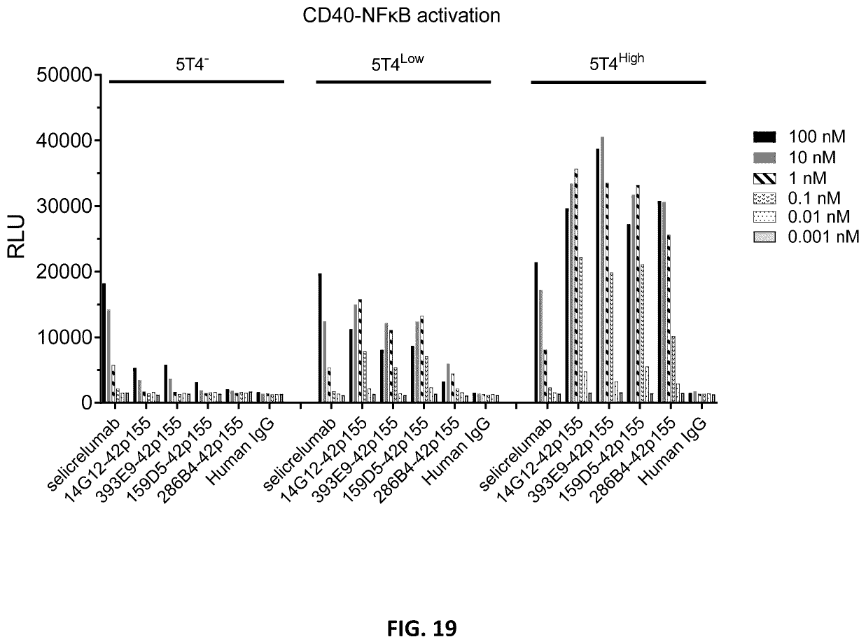

As shown in FIG. 19, the activity of the tested 5T4-CD40 bispecific antibodies with different epitopes in the 5T4 protein were solely or partly dependent on the expression of hu5T4 on the cells, showing much stronger activation of CD40 signaling in the presence of hu5T4. Moreover, 5T4-CD40 bispecific antibodies with 5T4 portion of bin A and bin B showed stronger agonist activity, especially when the target cells expressed moderate level of hu5T4 on surface.

### Activity of the 5T4-CD40 Bispecific Antibodies to Promote Human Dendritic Cell Immune Response

To investigate the ability of the 5T4-CD40 bispecific antibodies to stimulate human dendritic cell (DC) response, IL-12 cytokine release and CD80/CD86 expression of DCs were examined.

Human DCs were obtained following the procedures specified in Example 4. Human DCs were used as the effector cells. Cells expressing human 5T4 (MCF-7; 5T4^{Low} and CHO-K1-hu5T4: 5T4^{High}) or not (CHO-K1) were used as the target cells. Human DCs (5×10⁴) were co-cultured with target cells (1.5×10⁴) (E/T Ratio=˜3:1). Bispecific antibodies were 10-fold diluted serially and added to the culture medium at a final concentration starting from 100 nM. After incubation for 48 hours, the level of IL-12 in the culture medium was measured using IL-12/p40 (human) LANCE Ultra TR-FRET Detection Kit (PerkinElmer). Data were analyzed using a nonlinear regression, 4-parameter logistic equation.

Activation of DC leads to upregulation of costimulatory molecules CD80/CD86. Here, CD80/CD86 expression of DCs were examined by FACS following the staining and analysis procedures. Briefly, stimulated DCs were harvest by pipetting and washed by FACS buffer. After blocked with FcR block reagent (MACS) at 4° C. for 15 mins, PE Mouse anti-human CD80, BV421 Mouse anti-human CD86 and APC Mouse Anti-Human CD11c (BD) was added to each well and incubated at 4° C. for 30 minutes. After wash, MFI of PE and BV421 gated on CD11c+ cells were evaluated by MACSQuant Analyzer 16.

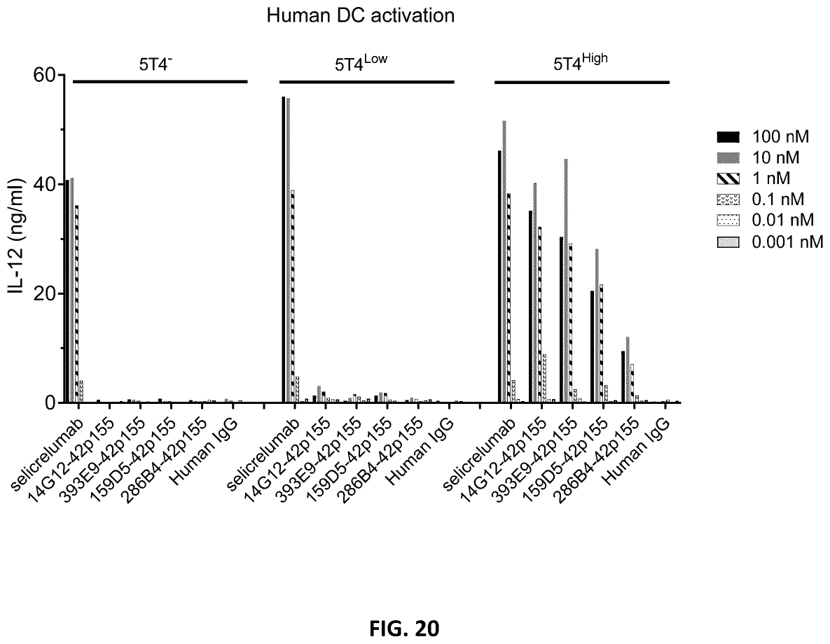

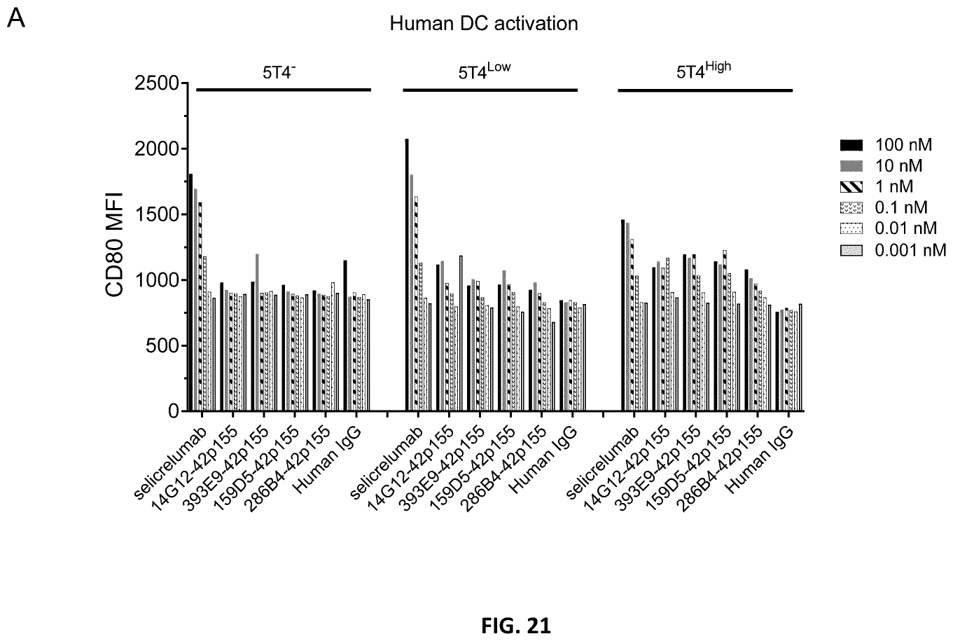

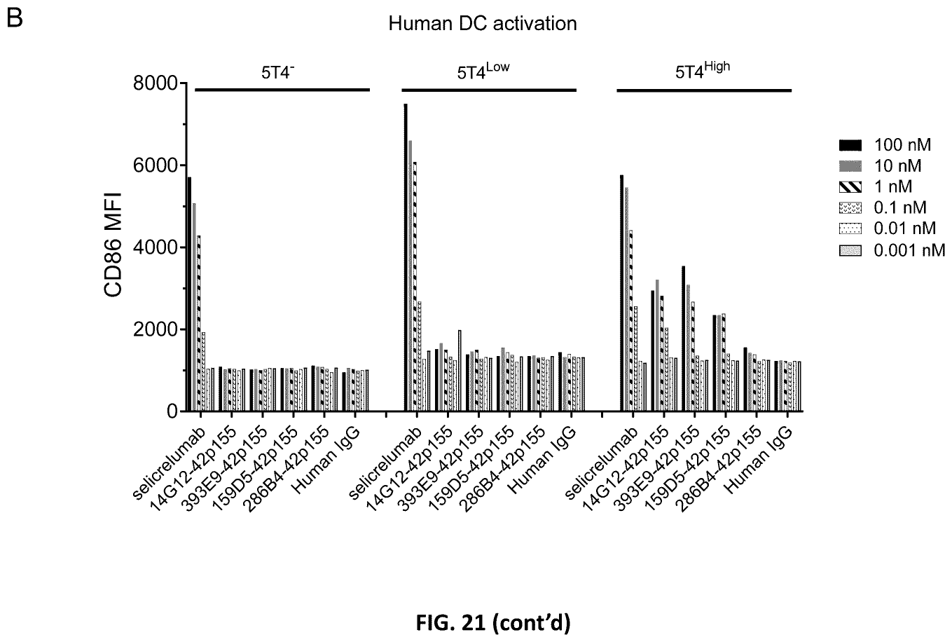

As shown in FIGS. 20 and 21, 5T4-CD40 bispecific antibodies could only activate DC response in the presence of hu5T4-expressing cells. And the potency was correlated with the expression level of hu5T4. Similarly, 5T4-CD40 bispecific antibodies with 5T4 portion of bin A and bin B showed stronger agonist activity.

### Example 13. Generation of Bispecific Antibodies with Different Bispecific Antibody Formats

To further activate CD40 pathway by 5T4-CD40 bispecific antibody, we have designed several bispecific antibody formats to determine the best format for 5T4-CD40. The configuration of the bispecific antibody with each pattern for individual target specificity were shown in FIG. 22. The peptide chains of different formats of bispecific antibodies are shown in Table. 12A, and the sequences of each target used in this format are shown in Table. 12B-C.

TABLE 12A
Peptide Chains of Example Bispecific Antibodies
b11 (42p155z2)

| | | | SEQ ID |
|---|---|---|---|
| | Name | Sequence | NO: |
| Heavy chain | 5T4 VH | EVQLQQSGPELVKPGASVKISCKASGYAFSSSWMNWVKQRP | 73 |
| | | GKGLEWIGRIYREDGHTNYNGKFKGKATLTADKSSTTAYMQ | |
| | | LTSLTSEDSAVYFCANGGAMDYWGQGTSVTVSS | |
| | CH (Human | ASTKGPSVFPLAPSSKSTSGGTAALGCLVKDYFPEPVTVSW | 158 |
| | IgG1 | NSGALTSGVHTFPAVLQSSGLYSLSSVVTVPSSSLGTQTYI | |
| | N297A) | CNVNHKPSNTKVDKKVEPKSCDKTHTCPPCPAPELLGGPSV | |
| | | FLFPPKPKDTLMISRTPEVTCVVVDVSHEDPEVKFNWYVDG | |
| | | VEVHNAKTKPREEQYASTYRVVSVLTVLHQDWLNGKEYKCK | |
| | | VSNKALPAPIEKTISKAKGQPREPQVYTLPPSREEMTKNQV | |
| | | SLTCLVKGFYPSDIAVEWESNGQPENNYKTTPPVLDSDGSF | |
| | | FLYSKLTVDKSRWQQGNVFSCSVMHEALHNHYTQKSLSLSP | |
| | | GK | |
| | Linker | GGGGSGGGGSGGGGSGGGGS | 159 |
| | CD40 VHH | EVQLVESGGGLVQPGGSLRLSCAASGFAFRSYTMSWVRQAP | 54 |
| | | GKGLEWVSTITHNGAITTYAESAQGRFTISRDNSKNTLYLQ | |
| | | MNSLRAEDTAVYYCAQGGGSNYYRESWGQGTQVTVSS | |
| Light chain | 5T4 VL | DIQMTQSTSSLSASLGDRVTISCRASQDISNYLNWYQQKPD | 74 |
| | | GTVKLLIYYTSRLHSGVPSRFSGSGSGTDYSLTISNLEQED | |
| | | IATYFCQQDNTLPWTFGGGTKLEIK | |
| | CL (human | RTVAAPSVFIFPPSDEQLKSGTASVVCLLNNFYPREAKVQW | 160 |
| | kappa) | KVDNALQSGNSQESVTEQDSKDSTYSLSSTLTLSKADYEKH | |
| | | KVYACEVTHQGLSSPVTKSFNRGEC | |

b12 (42p155z2)

| | | | SEQ ID |
|---|---|---|---|
| | Name | Sequence | NO: |
| Heavy chain 1 | 5T4 VH | EVQLQQSGPELVKPGASVKISCKASGYAFSSSWMNWVKQRP | 73 |
| | | GKGLEWIGRIYREDGHTNYNGKFKGKATLTADKSSTTAYMQ | |
| | | LTSLTSEDSAVYFCANGGAMDYWGQGTSVTVSS | |
| | CH (Human | ASTKGPSVFPLAPSSKSTSGGTAALGCLVKDYFPEPVTVSW | 161 |
| | IgG1 N297A | NSGALTSGVHTFPAVLQSSGLYSLSSVVTVPSSSLGTQTYI | |
| | Hole) | CNVNHKPSNTKVDKKVEPKSCDKTHTCPPCPAPELLGGPSV | |
| | | FLFPPKPKDTLMISRTPEVTCVVVDVSHEDPEVKFNWYVDG | |
| | | VEVHNAKTKPREEQYASTYRVVSVLTVLHQDWLNGKEYKCK | |
| | | VSNKALPAPIEKTISKAKGQPREPQVCTLPPSREEMTKNQV | |
| | | SLSCAVKGFYPSDIAVEWESNGQPENNYKTTPPVLDSDGSF | |
| | | FLVSKLTVDKSRWQQGNVFSCSVMHEALHNHYTQKSLSLSP | |
| | | GK | |
| Light chain | 5T4 VL | DIQMTQSTSSLSASLGDRVTISCRASQDISNYLNWYQQKPD | 74 |
| | | GTVKLLIYYTSRLHSGVPSRFSGSGSGTDYSLTISNLEQED | |
| | | IATYFCQQDNTLPWTFGGGTKLEIK | |
| | CL (human | RTVAAPSVFIFPPSDEQLKSGTASVVCLLNNFYPREAKVQW | 160 |
| | kappa) | KVDNALQSGNSQESVTEQDSKDSTYSLSSTLTLSKADYEKH | |
| | | KVYACEVTHQGLSSPVTKSFNRGEC | |
| Heavy chain 2 | CD40 VHH | EVQLVESGGGLVQPGGSLRLSCAASGFAFRSYTMSWVRQAP | 54 |
| | | GKGLEWVSTITHNGAITTYAESAQGRFTISRDNSKNTLYLQ | |
| | | MNSLRAEDTAVYYCAQGGGSNYYRESWGQGTQVTVSS | |
| | Fc (Human | EPKSADKTHTCPPCPAPELLGGPSVFLFPPKPKDTLMISRT | 162 |
| | IgG1 N297A | PEVTCVVVDVSHEDPEVKFNWYVDGVEVHNAKTKPREEQYA | |
| | Knob) | STYRVVSVLTVLHQDWLNGKEYKCKVSNKALPAPIEKTISK | |
| | | AKGQPREPQVYTLPPCREEMTKNQVSLWCLVKGFYPSDIAV | |
| | | EWESNGQPENNYKTTPPVLDSDGSFFLYSKLTVDKSRWQQG | |
| | | NVFSCSVMHEALHNHYTQKSLSLSPGK | |

b13 (42p155z2)

| | | | SEQ ID |
|---|---|---|---|
| | Name | Sequence | NO: |
| Heavy chain | 5T4 VH | EVQLQQSGPELVKPGASVKISCKASGYAFSSSWMNWVKQRP | 73 |
| | | GKGLEWIGRIYREDGHTNYNGKFKGKATLTADKSSTTAYMQ | |
| | | LTSLTSEDSAVYFCANGGAMDYWGQGTSVTVSS | |
| | CH (Human | ASTKGPSVFPLAPSSKSTSGGTAALGCLVKDYFPEPVTVSW | 158 |
| | IgG1 | NSGALTSGVHTFPAVLQSSGLYSLSSVVTVPSSSLGTQTYI | |
| | N297A) | CNVNHKPSNTKVDKKVEPKSCDKTHTCPPCPAPELLGGPSV | |
| | | FLFPPKPKDTLMISRTPEVTCVVVDVSHEDPEVKFNWYVDG | |
| | | VEVHNAKTKPREEQYASTYRVVSVLTVLHQDWLNGKEYKCK | |
| | | VSNKALPAPIEKTISKAKGQPREPQVYTLPPSREEMTKNQV | |
| | | SLTCLVKGFYPSDIAVEWESNGQPENNYKTTPPVLDSDGSF | |
| | | FLYSKLTVDKSRWQQGNVESCSVMHEALHNHYTQKSLSLSP | |
| | | GK | |
| Light chain | 5T4 VL | DIQMTQSTSSLSASLGDRVTISCRASQDISNYLNWYQQKPD | 74 |
| | | GTVKLLIYYTSRLHSGVPSRFSGSGSGTDYSLTISNLEQED | |
| | | IATYFCQQDNTLPWTFGGGTKLEIK | |
| | CL (human | RTVAAPSVFIFPPSDEQLKSGTASVVCLLNNFYPREAKVQW | 160 |
| | kappa) | KVDNALQSGNSQESVTEQDSKDSTYSLSSTLTLSKADYEKH | |
| | | KVYACEVTHQGLSSPVTKSFNRGEC | |
| | Linker | GGGGSGGGGSGGGGSGGGGS | 159 |
| | CD40 VHH | EVQLVESGGGLVQPGGSLRLSCAASGFAFRSYTMSWVRQAP | 54 |
| | | GKGLEWVSTITHNGAITTYAESAQGRFTISRDNSKNTLYLQ | |
| | | MNSLRAEDTAVYYCAQGGGSNYYRESWGQGTQVTVSS | |

b16 (42p155z2)

| | | | SEQ ID |
|---|---|---|---|
| | Name | Sequence | NO: |
| Heavy chain 1 | 5T4 VH | EVQLQQSGPELVKPGASVKISCKASGYAFSSSWMNWVKQRP | 73 |
| | | GKGLEWIGRIYREDGHTNYNGKFKGKATLTADKSSTTAYMQ | |
| | | LTSLTSEDSAVYFCANGGAMDYWGQGTSVTVSS | |
| | CH (Human | ASTKGPSVFPLAPSSKSTSGGTAALGCLVKDYFPEPVTVSW | 161 |
| | IgG1 N297A | NSGALTSGVHTFPAVLQSSGLYSLSSVVTVPSSSLGTQTYI | |
| | Hole) | CNVNHKPSNTKVDKKVEPKSCDKTHTCPPCPAPELLGGPSV | |
| | | FLFPPKPKDTLMISRTPEVTCVVVDVSHEDPEVKFNWYVDG | |
| | | VEVHNAKTKPREEQYASTYRVVSVLTVLHQDWLNGKEYKCK | |
| | | VSNKALPAPIEKTISKAKGQPREPQVCTLPPSREEMTKNQV | |
| | | SLSCAVKGFYPSDIAVEWESNGQPENNYKTTPPVLDSDGSE | |
| | | FLVSKLTVDKSRWQQGNVFSCSVMHEALHNHYTQKSLSLSP | |
| | | GK | |
| Light chain | 5T4 VL | DIQMTQSTSSLSASLGDRVTISCRASQDISNYLNWYQQKPD | 74 |
| | | GTVKLLIYYTSRLHSGVPSRFSGSGSGTDYSLTISNLEQED | |
| | | IATYFCQQDNTLPWTFGGGTKLEIK | |
| | CL (human | RTVAAPSVFIFPPSDEQLKSGTASVVCLLNNFYPREAKVQW | 160 |
| | kappa) | KVDNALQSGNSQESVTEQDSKDSTYSLSSTLTLSKADYEKH | |
| | | KVYACEVTHQGLSSPVTKSFNRGEC | |
| Heavy chain 2 | CD40 VHH | EVQLVESGGGLVQPGGSLRLSCAASGFAFRSYTMSWVRQAP | 54 |
| | | GKGLEWVSTITHNGAITTYAESAQGRFTISRDNSKNTLYLQ | |
| | | MNSLRAEDTAVYYCAQGGGSNYYRESWGQGTQVTVSS | |
| | Linker | GGGGSGGGGSGGGGS | 163 |
| | CD40 VHH | EVQLVESGGGLVQPGGSLRLSCAASGFAFRSYTMSWVRQAP | 54 |
| | | GKGLEWVSTITHNGAITTYAESAQGRFTISRDNSKNTLYLQ | |
| | | MNSLRAEDTAVYYCAQGGGSNYYRESWGQGTQVTVSS | |
| | Fc (Human | EPKSADKTHTCPPCPAPELLGGPSVFLFPPKPKDTLMISRT | 162 |
| | IgG1 N297A | PEVTCVVVDVSHEDPEVKFNWYVDGVEVHNAKTKPREEQYA | |
| | Knob) | STYRVVSVLTVLHQDWLNGKEYKCKVSNKALPAPIEKTISK | |
| | | AKGQPREPQVYTLPPCREEMTKNQVSLWCLVKGFYPSDIAV | |
| | | EWESNGQPENNYKTTPPVLDSDGSFFLYSKLTVDKSRWQQG | |
| | | NVFSCSVMHEALHNHYTQKSLSLSPGK | |

b17 (42p155z2)

| | | | SEQ ID |
|---|---|---|---|
| | Name | Sequence | NO: |
| Heavy chain 1 | 5T4 VH | EVQLQQSGPELVKPGASVKISCKASGYAFSSSWMNWVKQRP | 73 |
| | | GKGLEWIGRIYREDGHTNYNGKFKGKATLTADKSSTTAYMQ | |
| | | LTSLTSEDSAVYFCANGGAMDYWGQGTSVTVSS | |
| | CH (Human | ASTKGPSVFPLAPSSKSTSGGTAALGCLVKDYFPEPVTVSW | 161 |
| | IgG1 N297A | NSGALTSGVHTFPAVLQSSGLYSLSSVVTVPSSSLGTQTYI | |
| | Hole) | CNVNHKPSNTKVDKKVEPKSCDKTHTCPPCPAPELLGGPSV | |
| | | FLFPPKPKDTLMISRTPEVTCVVVDVSHEDPEVKFNWYVDG | |
| | | VEVHNAKTKPREEQYASTYRVVSVLTVLHQDWLNGKEYKCK | |
| | | VSNKALPAPIEKTISKAKGQPREPQVCTLPPSREEMTKNQV | |
| | | SLSCAVKGFYPSDIAVEWESNGQPENNYKTTPPVLDSDGSF | |
| | | FLVSKLTVDKSRWQQGNVESCSVMHEALHNHYTQKSLSLSP | |
| | | GK | |
| | Linker | GGGGSGGGGSGGGGSGGGGS | 159 |
| | CD40 VHH | EVQLVESGGGLVQPGGSLRLSCAASGFAFRSYTMSWVRQAP | 54 |
| | | GKGLEWVSTITHNGAITTYAESAQGRFTISRDNSKNTLYLQ | |
| | | MNSLRAEDTAVYYCAQGGGSNYYRESWGQGTQVTVSS | |
| Light chain | 5T4 VL | DIQMTQSTSSLSASLGDRVTISCRASQDISNYLNWYQQKPD | 74 |
| | | GTVKLLIYYTSRLHSGVPSRFSGSGSGTDYSLTISNLEQED | |
| | | IATYFCQQDNTLPWTFGGGTKLEIK | |
| | CL (human | RTVAAPSVFIFPPSDEQLKSGTASVVCLLNNFYPREAKVQW | 160 |
| | kappa) | KVDNALQSGNSQESVTEQDSKDSTYSLSSTLTLSKADYEKH | |
| | | KVYACEVTHQGLSSPVTKSFNRGEC | |
| Heavy chain 2 | Fc (Human | EPKSADKTHTCPPCPAPELLGGPSVFLFPPKPKDTLMISRT | 162 |
| | IgG1 N297A | PEVTCVVVDVSHEDPEVKFNWYVDGVEVHNAKTKPREEQYA | |
| | Knob) | STYRVVSVLTVLHQDWLNGKEYKCKVSNKALPAPIEKTISK | |
| | | AKGQPREPQVYTLPPCREEMTKNQVSLWCLVKGFYPSDIAV | |
| | | EWESNGQPENNYKTTPPVLDSDGSFFLYSKLTVDKSRWQQG | |
| | | NVFSCSVMHEALHNHYTQKSLSLSPGK | |
| | Linker | GGGGSGGGGSGGGGSGGGGS | 159 |
| | CD40 VHH | EVQLVESGGGLVQPGGSLRLSCAASGFAFRSYTMSWVRQAP | 54 |
| | | GKGLEWVSTITHNGAITTYAESAQGRFTISRDNSKNTLYLQ | |
| | | MNSLRAEDTAVYYCAQGGGSNYYRESWGQGTQVTVSS | |

b18 (2p1130)

| | | | SEQ ID |
|---|---|---|---|
| | Name | Sequence | NO: |
| Heavy chain 1 | 5T4 VH | EVQLQQSGPELVKPGASVKISCKASGYAFSSSWMNWVKQRP | 73 |
| | | GKGLEWIGRIYREDGHTNYNGKFKGKATLTADKSSTTAYMQ | |
| | | LTSLTSEDSAVYFCANGGAMDYWGQGTSVTVSS | |
| | CH (Human | ASTKGPSVFPLAPSSKSTSGGTAALGCLVKDYFPEPVTVSW | 161 |
| | IgG1 N297A | NSGALTSGVHTFPAVLQSSGLYSLSSVVTVPSSSLGTQTYI | |
| | Hole) | CNVNHKPSNTKVDKKVEPKSCDKTHTCPPCPAPELLGGPSV | |
| | | FLFPPKPKDTLMISRTPEVTCVVVDVSHEDPEVKFNWYVDG | |
| | | VEVHNAKTKPREEQYASTYRVVSVLTVLHQDWLNGKEYKCK | |
| | | VSNKALPAPIEKTISKAKGQPREPQVCTLPPSREEMTKNQV | |
| | | SLSCAVKGFYPSDIAVEWESNGQPENNYKTTPPVLDSDGSF | |
| | | FLVSKLTVDKSRWQQGNVFSCSVMHEALHNHYTQKSLSLSP | |
| | | GK | |
| Light chain | 5T4 VL | DIQMTQSTSSLSASLGDRVTISCRASQDISNYLNWYQQKPD | 74 |
| | | GTVKLLIYYTSRLHSGVPSRFSGSGSGTDYSLTISNLEQED | |
| | | IATYFCQQDNTLPWTFGGGTKLEIK | |
| | CL (human | RTVAAPSVFIFPPSDEQLKSGTASVVCLLNNFYPREAKVQW | 160 |
| | kappa) | KVDNALQSGNSQESVTEQDSKDSTYSLSSTLTLSKADYEKH | |
| | | KVYACEVTHQGLSSPVTKSFNRGEC | |
| Heavy chain 2 | CD40 VHH | EVQVVESGGGLVQPGGSLRLTCAASGFSVDDYGIGWERQAP | 13 |
| | | GREREGVACITPNGLMMNFANTVGSVAGRESASRDSGENTV | |
| | | SLQMSSLKPEDTAIYFCAHSRDDSCRGSLSDYDDWGQGTQV | |
| | | TVSS | |
| | Linker | GGGGSGGGGSGGGGS | 163 |
| | CD40 VHH | EVQVVESGGGLVQPGGSLRLTCAASGFSVDDYGIGWERQAP | 13 |
| | | GREREGVACITPNGLMMNFANTVGSVAGRESASRDSGENTV | |
| | | SLQMSSLKPEDTAIYFCAHSRDDSCRGSLSDYDDWGQGTQV | |
| | | TVSS | |
| | Linker | GGGGSGGGGSGGGGS | 163 |
| | CD40 VHH | EVQVVESGGGLVQPGGSLRLTCAASGFSVDDYGIGWFRQAP | 13 |
| | | GREREGVACITPNGLMMNFANTVGSVAGRESASRDSGENTV | |
| | | SLQMSSLKPEDTAIYFCAHSRDDSCRGSLSDYDDWGQGTQV | |
| | | TVSS | |
| | Fc (Human | EPKSADKTHTCPPCPAPELLGGPSVFLFPPKPKDTLMISRT | 162 |
| | IgG1 N297A | PEVTCVVVDVSHEDPEVKFNWYVDGVEVHNAKTKPREEQYA | |
| | Knob) | STYRVVSVLTVLHQDWLNGKEYKCKVSNKALPAPIEKTISK | |
| | | AKGQPREPQVYTLPPCREEMTKNQVSLWCLVKGFYPSDIAV | |
| | | EWESNGQPENNYKTTPPVLDSDGSFFLYSKLTVDKSRWQQG | |
| | | NVFSCSVMHEALHNHYTQKSLSLSPGK | |

b16 (42p155z2)-LALA

| | | | SEQ ID |
|---|---|---|---|
| | Name | Sequence | NO: |
| Heavy chain 1 | 5T4 VH | QVQLVQSGAEVKKPGASVKVSCKASGYAFSSSWMNWVRQAP | 83 |
| | | GQGLEWMGRIYREDGHTNYNGKFKGRVTMTADKSITTAYME | |
| | | LSRLRSDDTAVYYCANGGAMDYWGQGTLVTVSS | |
| | CH (Human | ASTKGPSVFPLAPSSKSTSGGTAALGCLVKDYFPEPVTVSW | 164 |
| | IgG1 LALA | NSGALTSGVHTFPAVLQSSGLYSLSSVVTVPSSSLGTQTYI | |
| | Hole) | CNVNHKPSNTKVDKKVEPKSCDKTHTCPPCPAPEAAGGPSV | |
| | | FLFPPKPKDTLMISRTPEVTCVVVDVSHEDPEVKFNWYVDG | |
| | | VEVHNAKTKPREEQYNSTYRVVSVLTVLHQDWLNGKEYKCK | |
| | | VSNKALPAPIEKTISKAKGQPREPQVCTLPPSREEMTKNQV | |
| | | SLSCAVKGFYPSDIAVEWESNGQPENNYKTTPPVLDSDGSF | |
| | | FLVSKLTVDKSRWQQGNVESCSVMHEALHNHYTQKSLSLSP | |
| | | GK | |
| Light chain | 5T4 VL | DIQMTQSPSSLSASVGDRVTITCRASQDISNYLNWYQQKPG | 91 |
| | | KAPKLLIYYTSRLHSGVPSRFSGSGSGTDYTFTISSLQPED | |
| | | IATYFCQQDNTLPWTFGGGTKVEIK | |
| | CL (human | RTVAAPSVFIFPPSDEQLKSGTASVVCLLNNFYPREAKVQW | 160 |
| | kappa) | KVDNALQSGNSQESVTEQDSKDSTYSLSSTLTLSKADYEKH | |
| | | KVYACEVTHQGLSSPVTKSFNRGEC | |
| Heavy chain 2 | CD40 VHH | EVQLVESGGGLVQPGGSLRLSCAASGFAFRSYTMSWVRQAP | 54 |
| | | GKGLEWVSTITHNGAITTYAESAQGRFTISRDNSKNTLYLQ | |
| | | MNSLRAEDTAVYYCAQGGGSNYYRESWGQGTQVTVSS | |
| | Linker | GGGGSGGGGSGGGGS | 163 |
| | CD40 VHH | EVQLVESGGGLVQPGGSLRLSCAASGFAFRSYTMSWVRQAP | 54 |
| | | GKGLEWVSTITHNGAITTYAESAQGRETISRDNSKNTLYLQ | |
| | | MNSLRAEDTAVYYCAQGGGSNYYRESWGQGTQVTVSS | |
| | Fc (Human | EPKSADKTHTCPPCPAPEAAGGPSVFLFPPKPKDTLMISRT | 165 |
| | IgG1 LALA | PEVTCVVVDVSHEDPEVKFNWYVDGVEVHNAKTKPREEQYN | |
| | Knob) | STYRVVSVLTVLHQDWLNGKEYKCKVSNKALPAPIEKTISK | |
| | | AKGQPREPQVYTLPPCREEMTKNQVSLWCLVKGFYPSDIAV | |
| | | EWESNGQPENNYKTTPPVLDSDGSFFLYSKLTVDKSRWQQG | |
| | | NVFSCSVMHEALHNHYTQKSLSLSPGK | |

TABLE 12B
Antibody Variable Region Sequences of CD40 portion

| | | SEQ ID |
|---|---|---|
| Name | Sequence | NO: |
| 42p155z2 | EVQLVESGGGLVQPGGSLRLSCAASGFAFRSYTMSWVRQAPGKGLEW | 54 |
| | VSTITHNGAITTYAESAQGRFTISRDNSKNTLYLQMNSLRAEDTAVY | |
| | YCAQGGGSNYYRESWGQGTQVTVSS | |
| 2p1130 | EVQVVESGGGLVQPGGSLRLTCAASGFSVDDYGIGWERQAPGREREG | 13 |
| | VACITPNGLMMNFANTVGSVAGRFSASRDSGENTVSLQMSSLKPEDT | |
| | AIYFCAHSRDDSCRGSLSDYDDWGQGTQVTVSS | |

TABLE 12C
Antibody Variable Region Sequences of 5T4 portion Used/Suitable for Use in
Bispecific Antibodies

| | | SEQ ID |
|---|---|---|
| Name | Sequence | NO: |
| 14G12 VH | EVQLQQSGPELVKPGASVKISCKASGYAFSSSWMNWVKQRPGKGLEW | 73 |
| | IGRIYREDGHTNYNGKFKGKATLTADKSSTTAYMQLTSLTSEDSAVY | |
| | FCANGGAMDYWGQGTSVTVSS | |
| 14G12 VL | DIQMTQSTSSLSASLGDRVTISCRASQDISNYLNWYQQKPDGTVKLL | 74 |
| | IYYTSRLHSGVPSRFSGSGSGTDYSLTISNLEQEDIATYFCQQDNTL | |
| | PWTFGGGTKLEIK | |
| 14G12 hVH2 | QVQLVQSGAEVKKPGASVKVSCKASGYAFSSSWMNWVRQAPGQGLEW | 83 |
| | MGRIYREDGHTNYNGKFKGRVTMTADKSITTAYMELSRLRSDDTAVY | |
| | YCANGGAMDYWGQGTLVTVSS | |
| 14G12 hVL1 | DIQMTQSPSSLSASVGDRVTITCRASQDISNYLNWYQQKPGKAPKLL | 91 |
| | IYYTSRLHSGVPSRFSGSGSGTDYTFTISSLQPEDIATYFCQQDNTL | |
| | PWTFGGGTKVEIK | |
| 14G12 hVH5 | QVQLVQSGAEVKKPGASVKVSCKASGYAFSSSWMNWVRQAPGQGLEW | 89 |
| | MGRIYREDGHTNYNGKFKGRVTMTADKSTTTAYMELRSLRSDDTAVY | |
| | YCANGGAMDYWGQGTLVTVSS | |
| 14G12 hVL1 | DIQMTQSPSSLSASVGDRVTITCRASQDISNYLNWYQQKPGKAPKLL | 91 |
| | IYYTSRLHSGVPSRFSGSGSGTDYTFTISSLQPEDIATYFCQQDNTL | |
| | PWTFGGGTKVEIK | |
| 393E9 VH.V4 | EVQLVESGGGLVQPGGSLRLSCAASGETFTDYYMSWVRQAPGKGLEW | 113 |
| | VGFIRNKGNGYTTENSASVKGRFTISRDNSKSSLYLQMNSLKTEDTA | |
| | VYYCARYRGNPHYAMDYWGQGTLVTVSS | |
| 393E9 VL.V5 | EIVLTQSPGTLSLSPGERATLSCRASESVEFYGTSFLQWYQQKPGQA | 120 |
| | PRLLIYGASNVESGIPDRFSGSGSGTDETLTISRLEPEDIAVYFCQQ | |
| | SRKVPSTFGGGTKVEIK | |
| 159D5 VH.V0 | EVQLVESGGGVVQPGRSLRLSCAASGETFSNFGMHWVRQAPGKGLEW | 130 |
| | VAYISSGSSTFYYADSVKGRFTISRDNSKNTLYLQMNSLRAEDTAVY | |
| | YCARSPAYYRYGLDYWGQGTTVTVSS | |
| 159D5 VL.V1 | DIVMTQTPLSLSVTPGQPASISCRASQSVSSSTESYMHWYQQKPGQP | 133 |
| | PQLLIKSSSNLESGVPDRFSGSGSGTDFTLKISRVEAEDVGVYYCQH | |
| | SWEIPYTFGGGTKVEIK | |

### Example 14. Selection of 5T4-CD40 Format with Different CD40 Activities

To select the suitable format for 5T4-CD40 bispecific antibodies, we tested the functional activities of 5T4-CD40 bispecific antibodies with different formats and CD40) activities to select the most potent and clean combination for 5T4-CD40 bispecific antibodies.

### Binding Activity of the Bispecific Antibodies to Human CD40 on Cell Surface

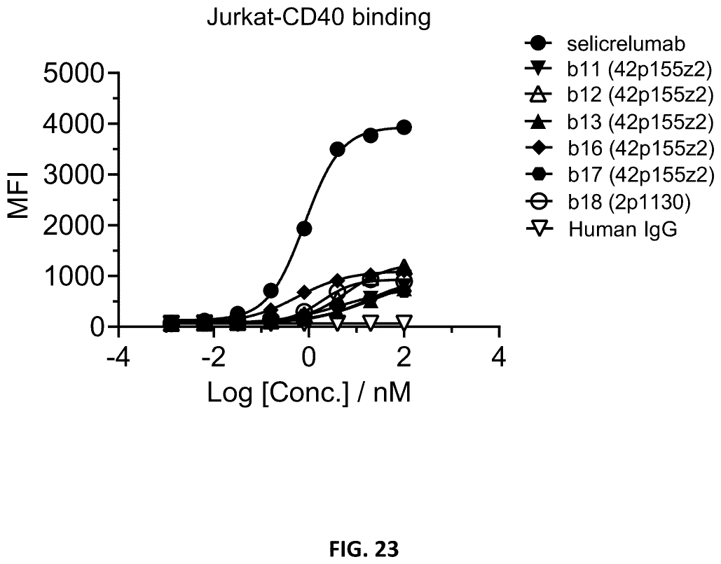

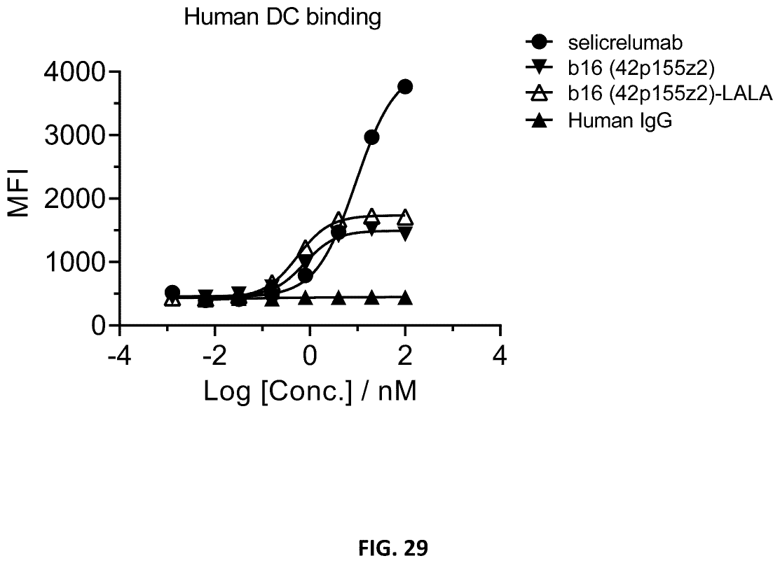

To evaluate the binding affinity of 5T4-CD40 bispecific antibodies to cell surface CD40, bispecific antibodies with formats of b11 (42p15522), b12 (42p155/2), b13 (42p15522), b16 (42p155×2), b17 (42p155/2) and b18 (2p1130) were tested in Jurkat cell lines that overexpressed CD40 by FACS. A total number of 1×10⁵ Jurkat-CD40 cells in each well were incubated with 5-fold serially diluted antibodies starting from 100 nM for 30 minutes at 4° C. in FACS buffer. After wash by FACS buffer, PE conjugated-anti-human IgG antibody was added to each well and incubated at 4° C. for 30 minutes. After wash, MFI of PE was evaluated by MACSQuant Analyzer 16. As shown in FIG. 23, the binding activity of the tested 5T4-CD40 bispecific antibodies depended on the antibody format as well as on the CD40 clone. Moreover, as shown in FIG. 29, humanized version of 5T4-CD40 biAb, b16 (42p15522)-LALA showed comparable binding to its chimeric antibody b16 (42p155/2), against CD40 expression on the DC.

Cell-Line Based Functional Characterization of 5T4-CD40 Bispecific Antibodies with Different Formats and CD40 Clones

To assess the potency of the 5T4-CD40 bispecific antibodies to activate CD40 signaling pathway, a commercial CD40 NF-κB luciferase reporter system was used. In this assay, H_CD40(TNFRSF5) NFκB-Reporter Jurkat (Genomeditech, cat #GM-C09520) was used as effector cells, and cells expressing human 5T4 (MCF-7: 5T4^{Low} and CHO-K1-hu5T4; 5T4^{High}) or not (CHO-K1) were used as target cells. Briefly, effector cells at a density of 2.0×10⁴ cells per well were cocultured with 2.0×10⁴ target cells (E/T Ratio=1:1) in a white 96-well plate. Antibodies were 10-fold serially diluted and added to a white 96-well assay plate, at a final concentration ranging from 0.001 nM to 100 nM. After 5 hours incubation at 37° C., luminescence was obtained by adding the substrate of luciferase and measured by a microplate reader. Four-parameter logistic curve analysis was performed with GraphPad software.

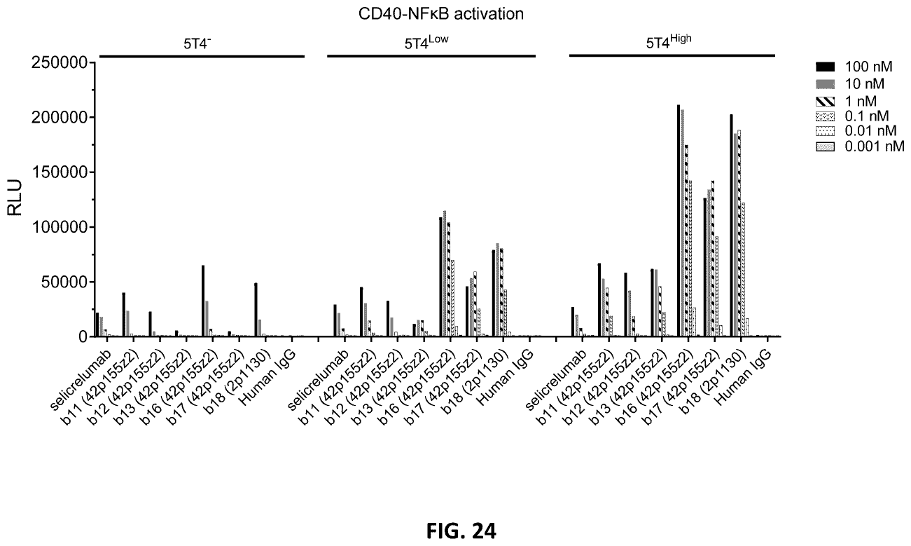

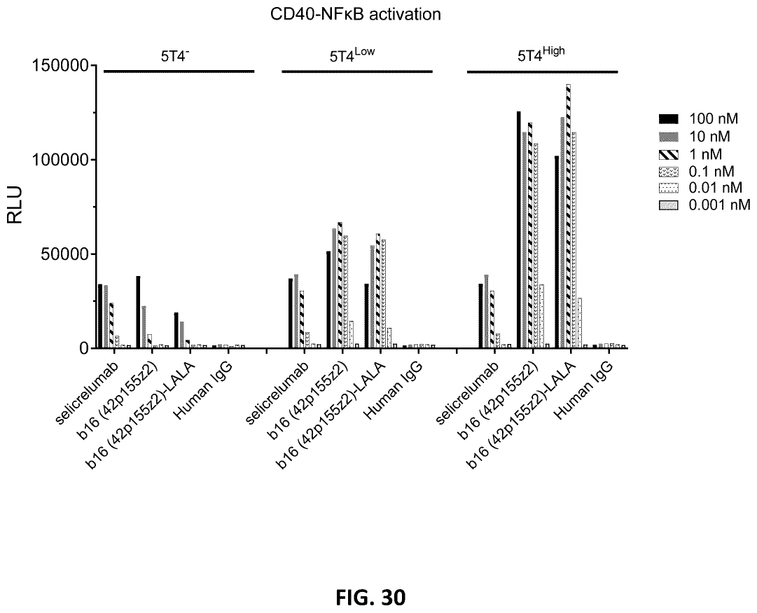

As shown in FIG. 24, the activity of the tested 5T4-CD40 bispecific antibodies with different formats and CD40 clones were solely or partly dependent on the expression of hu5T4 on the cells, showing much stronger activation of CD40 signaling in the presence of hu5T4. Moreover, the activity of the test 5T4-CD40 bispecific antibodies were significantly different, and bispecific antibodies with b16 and b18 formats showed much stronger activity than other formats. Furthermore, as shown in FIG. 30, humanized version of 5T4-CD40 biAb, b16 (42p155z2)-LALA showed comparable activity to its chimeric antibody b16 (42p155z2), in inducing CD40 downstream NF-κB signaling when there is expression of 5T4.

### Activity of the 5T4-CD40 Bispecific Antibodies to Promote Human Dendritic Cell Immune Response

To investigate the ability of the 5T4-CD40 bispecific antibodies to stimulate human dendritic cell (DC) response, IL-12 cytokine release of DCs were examined.

Human DCs were obtained following the procedures specified in Example 4. Human DCs were used as the effector cells. Cells expressing human 5T4 (MCF-7: 5T4^{Low} and CHO-K1-hu5T4: 5T4^{High}) or not (CHO-K1) were used as the target cells. Human DCs (5×10⁴) were co-cultured with target cells (1.5×10⁴) (E/T Ratio=˜3:1). Bispecific antibodies were 10-fold diluted serially and added to the culture medium at a final concentration starting from 100 nM. After incubation for 48 hours, the level of IL-12 in the culture medium was measured using IL-12/p40 (human) LANCE Ultra TR-FRET Detection Kit (PerkinElmer). Data were analyzed using a nonlinear regression, 4-parameter logistic equation.

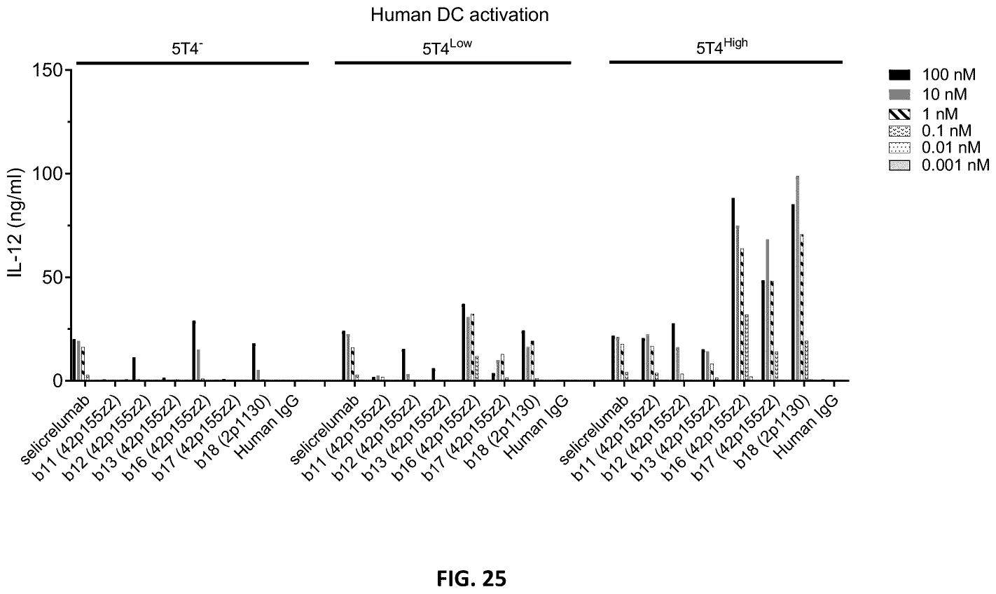

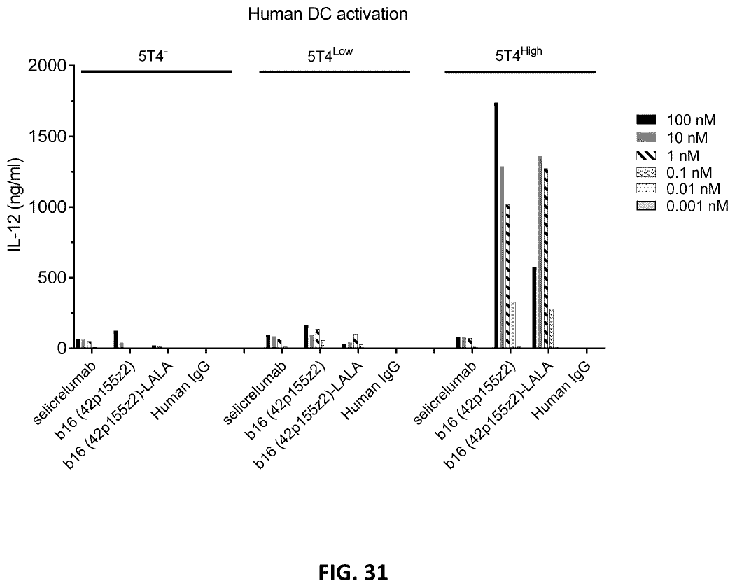

As shown in FIG. 25, the activity of the tested 5T4-CD40 bispecific antibodies with different formats and CD40 clones were solely or partly dependent on the expression of hu5T4 on the cells, showing much stronger activation of CD40 signaling in the presence of hu5T4. Similarly, the potency of the test 5T4-CD40 bispecific antibodies varied significantly, and bispecific antibodies with b16 and b18 formats showed much stronger agonist activity than others. Furthermore, as shown in FIG. 31, humanized version of 5T4-CD40 biAb, b16 (42p155z2)-LALA showed comparable activity to its chimeric antibody b16 (42p155z2) in inducing IL2 secretion from DC, when there is expression of 5T4.

### Example 15. Tumor Growth Inhibition by 5T4-CD40 Bispecific Antibodies

In this example, humanized mice that the extracellular domain of mouse CD40 were replaced by human counterpart CD40, were used to test the anti-tumor activity of the 5T4-CD40 bispecific antibodies.

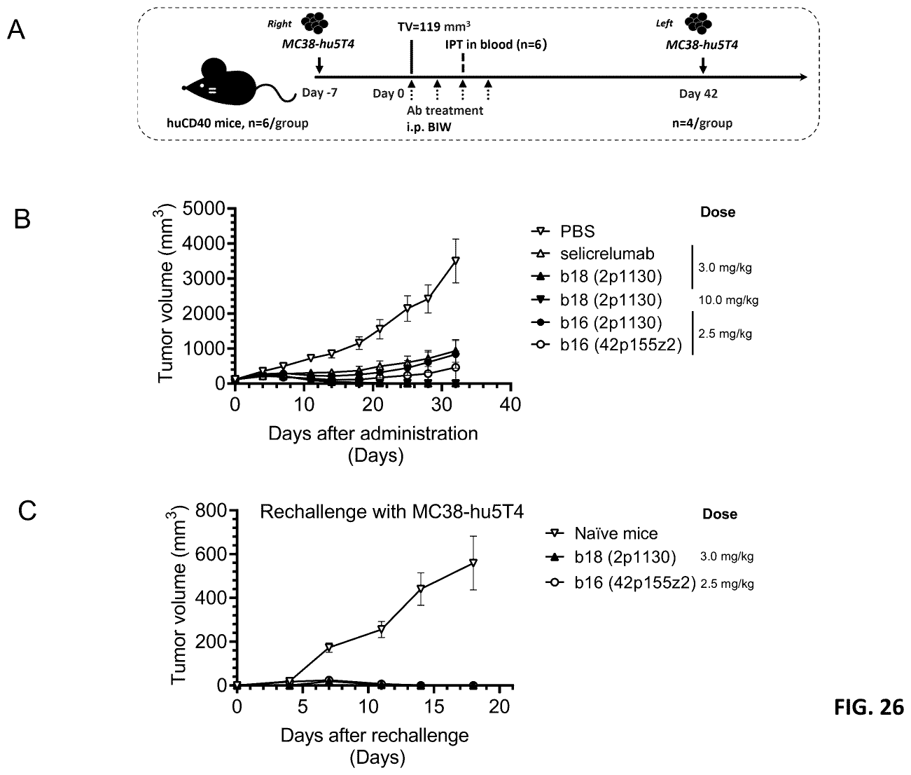

Mouse colon adenocarcinoma cells (MC38) were engineered to express human 5T4 (MC38-hu5T4). Humanized C57BL/6 mice (huCD40) were subcutaneously implanted with MC38-hu5T4 cells. As shown in FIG. 26A, mice were intraperitoneally administered twice a week for 4 times with following antibodies: PBS control, selicrelumab (3 mg/kg), b18 (2p1130) (3 mg/kg), b18 (2p1130) (10 mg/kg), b16 (2p1130) (2.5 mg/kg) and b16 (42p155×2) (2.5 mg/kg). All the test antibodies were administered in equal molar amount.

As shown in FIG. 26B and Table 13, mice treated with selicrelumab and b16 (2p1130) only showed a moderated response. Selicrelumab can suppress the tumor growth with the TGI of 76.1% and b16 (2p1130) with the TGI of 83.8% at Day 25. Meanwhile, b18 (2p1130) (3 mg/kg) and b16 (42p15522) induced a complete tumor remission in 4/6 and 5/6 mice respectively since Day 39. And all mice treated with b18 (2p1130) (10 mg/kg) had complete tumor regression since Day 35. These results indicate a strong anti-tumor efficacy and tumor growth regression with the 5T4 targeted-CD40 therapy in contrast to the non-targeted CD40 therapy. To determine whether CD40-targeted therapy could induce an immune memory, mice with complete tumor remission were given a second challenge of MC38-hu5T4 in the opposite flank. As shown in FIG. 26C, all these mice were resistant to the tumor rechallenge, suggesting the durable immune memory response was established in mice treated with 5T4-CD40 bispecific antibodies b18 (2p1130) and b16 (42p15522).

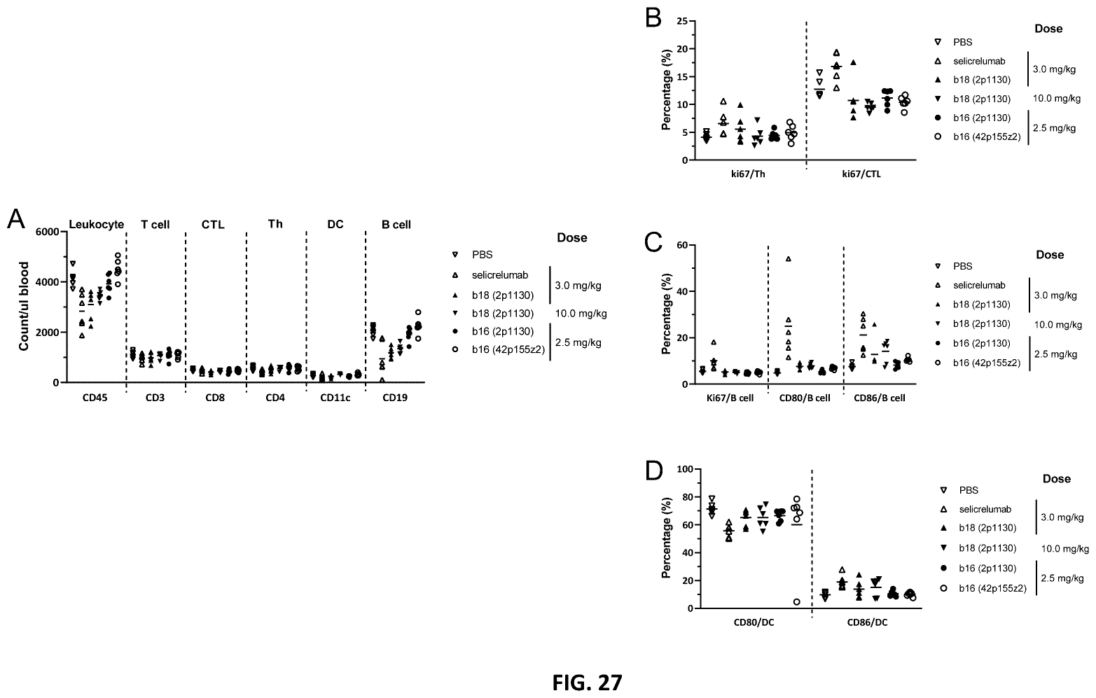

The immunophenotyping (IPT) analysis in the peripheral blood was performed on Day 7 after the first administration. As shown in FIGS. 27, b16 (2p1130) and b16 (42p15522) didn't change the cell count of B cells, compared to the PBS group, while selicrelumab and b18 (2p1 130) decreased the cell number (FIG. 27A). Furthermore, selicrelumab also increased the expression of CD80 and CD86 in B cells and proliferation of T cells significantly, while other bispecific antibodies didn't change or only slightly increased the activation marker (FIG. 27B-C). These data suggest that b16 (2p1130) and b16 (42p15522) did not active the immune cells in the peripheral.

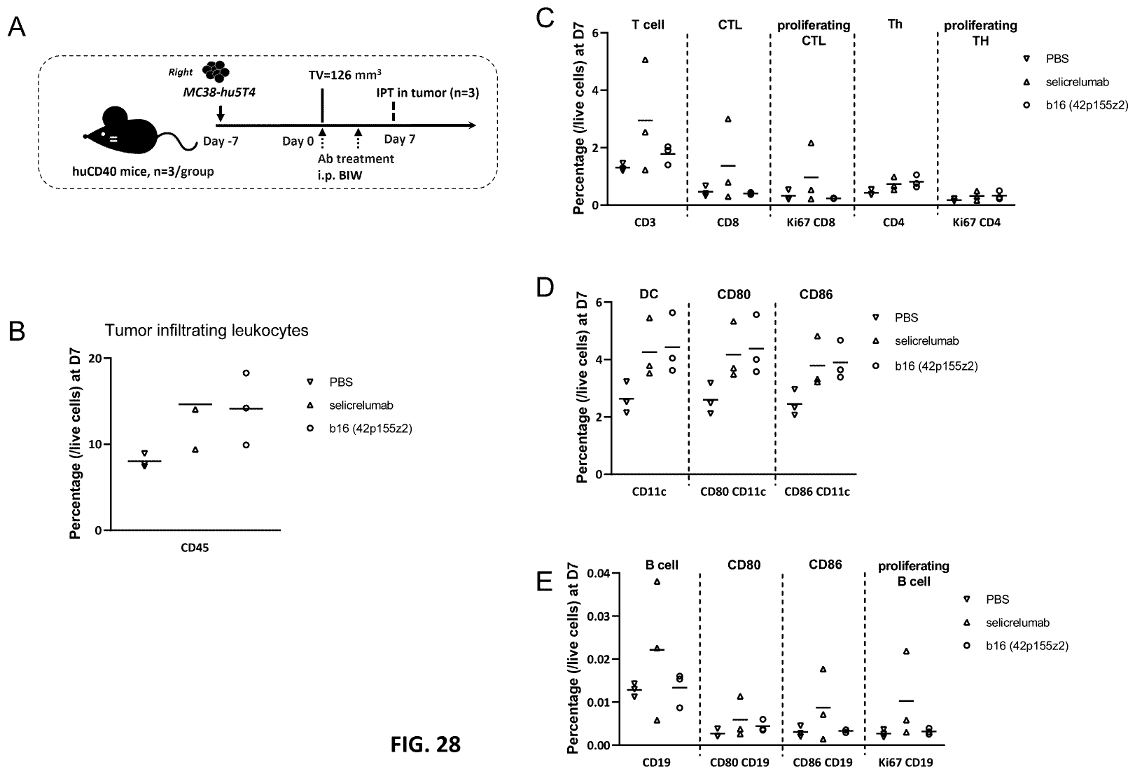

To assess the immune activation upon CD40-based therapy, tumor infiltrating IPT analysis was performed on Day 7 in a separate group with the same experiment design (FIG. 28A). As shown in FIG. 28B-E, b16 (42p155z2) induced immune response in tumor tissue, including increase in percentage of leukocytes, CD4+ T cells and DCs, compared to PBS group. Moreover, b16 (42p155z2) also increased the expression of CD80 and CD86 in DCs, compared to PBS group. These data suggest that 5T4-CD40 bispecific antibody was a promising therapeutic approach for 5T4-expressing tumor via 5T4-dependent activation of CD40.

TABLE 13
TGI of MC38-hu5T4 mouse model

| Group | TGI (%) at D 25 |
|---|---|
| selicrelumab (3 mpk) | 76.1% |
| b18 (2p1130) (3 mpk) | 105.3% |
| b18 (2p1130) (10 mpk) | 105.2% |
| b16 (2p1130) (2.5 mpk) | 83.8% |
| b16 (42p155z2) (2.5 mpk) | 94.2% |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A method of treating cancer in a patient in need thereof, comprising administering to the patient an effective amount of a single domain antibody or a polypeptide comprising the single domain antibody, wherein the single domain antibody has binding specificity to the human cluster of differentiation 40 (CD40) protein and comprises a complementarity determining region 1 (CDR1), a CDR2 and a CDR3, wherein the CDR1, CDR2 and CDR3 comprise, respectively,
(1) the amino acid sequences of SEQ ID NO: 14, 15 and 16;
(1a) the amino acid sequences of SEQ ID NO: 14, 63 and 16;
(1b) the amino acid sequences of SEQ ID NO: 14, 64 and 16;
(2) the amino acid sequences of SEQ ID NO: 17, 18 and 19;
(3) the amino acid sequences of SEQ ID NO: 20, 21 and 22;
(4) the amino acid sequences of SEQ ID NO: 23, 24 and 25;
(5) the amino acid sequences of SEQ ID NO: 26, 27 and 28;
(6) the amino acid sequences of SEQ ID NO: 29, 30 and 31;
(7) the amino acid sequences of SEQ ID NO: 32, 33 and 34;
(8) the amino acid sequences of SEQ ID NO: 35, 36 and 37;
(9) the amino acid sequences of SEQ ID NO: 38, 39 and 40;
(10) the amino acid sequences of SEQ ID NO: 41, 42 and 43;
(11) the amino acid sequences of SEQ ID NO: 44, 45 and 46;
(12) the amino acid sequences of SEQ ID NO: 47, 48 and 49; or
(13) the amino acid sequences of SEQ ID NO: 50, 51 and 52.

2. The method of claim 1, wherein the CDR1, CDR2 and CDR3 comprise, respectively,
(1) the amino acid sequences of SEQ ID NO: 14, 15 and 16;
(1a) the amino acid sequences of SEQ ID NO: 14, 63 and 16;
(1b) the amino acid sequences of SEQ ID NO: 14, 64 and 16;
(2) the amino acid sequences of SEQ ID NO: 17, 18 and 19;
(3) the amino acid sequences of SEQ ID NO: 20, 21 and 22;
(4) the amino acid sequences of SEQ ID NO: 23, 24 and 25;
(5) the amino acid sequences of SEQ ID NO: 26, 27 and 28; or
(10) the amino acid sequences of SEQ ID NO: 41, 42 and 43.

3. The method of claim 2, wherein the CDR1 comprises the amino acid sequence of SEQ ID NO:14, the CDR2 comprises the amino acid sequence of SEQ ID NO: 15, 63 or 64, and the CDR3 comprises the amino acid sequence of SEQ ID NO: 16.

4. The method of claim 3, wherein the CDR1 comprises the amino acid sequence of SEQ ID NO: 14, the CDR2 comprises the amino acid sequence of SEQ ID NO: 15, and the CDR3 comprises the amino acid sequence of SEQ ID NO:16.

5. The method of claim 4, which comprises an amino acid sequence selected from the group consisting of SEQ ID NO: 53, 54, 57 and 60.

6. The method of claim 3, wherein the CDR1 comprises the amino acid sequence of SEQ ID NO: 14, the CDR2 comprises the amino acid sequence of SEQ ID NO:63, and the CDR3 comprises the amino acid sequence of SEQ ID NO:16.

7. The method of claim 6, which comprises an amino acid sequence selected from the group consisting of SEQ ID NO: 55, 58 and 61.

8. The method of claim 3, wherein the CDR1 comprises the amino acid sequence of SEQ ID NO:14, the CDR2 comprises the amino acid sequence of SEQ ID NO:64, and the CDR3 comprises the amino acid sequence of SEQ ID NO: 16.

9. The method of claim 8, which comprises an amino acid sequence selected from the group consisting of SEQ ID NO: 56, 59 and 62.

10. The method of claim 2, wherein the CDR1 comprises the amino acid sequence of SEQ ID NO:17, the CDR2 comprises the amino acid sequence of SEQ ID NO:18, and the CDR3 comprises the amino acid sequence of SEQ ID NO:19.

11. The method of claim 10, which comprises an amino acid sequence selected from the group consisting of SEQ ID NO:65-68.

12. The method of claim 2, wherein the CDR1 comprises the amino acid sequence of SEQ ID NO:41, the CDR2 comprises the amino acid sequence of SEQ ID NO:42, and the CDR3 comprises the amino acid sequence of SEQ ID NO:43.

13. The method of claim 12, which comprises an amino acid sequence selected from the group consisting of SEQ ID NO:69-72.

14. The method of claim 2, which comprises the amino acid sequence of SEQ ID NO:3, 4 or 5.

15. The method of claim 1, further comprising administration of an immune checkpoint inhibitor.


SEQUENCE LISTING (165 sequences)

SEQ ID NO: 1 - protein, 119 aa, synthetic construct
PSQLVESGGGLVQPGGSLRLSCAASGFAFRSYTMSWVRQAPGKGLEWVSTITHNGAITTYAESAQGRFTISRDNAKNTLYLQMNSLKPEDTAVYYCAQGGGSNYYRESWGQGTQVTVSS

SEQ ID NO: 2 - protein, 119 aa, synthetic construct
QSQLVESGGGLVQPGGSLRLSCAASGFAFRRYTMSWVRQAPGKGLEWVSTITHNGSITTYAESAQGRFTISRDNAKNTLYLQMNSLKPEDTAVYYCAQGGGSNYYRESWGQGTQVTVSS

SEQ ID NO: 3 - protein, 119 aa, synthetic construct
AVQLVESGGGLVQPGGSLRLSCAASGFSFRNYMMNWVRQAPGKGLECLSSITSAGDITSYTESVKGRFTISRDNAKNTLYLQMNSLKPEDTAVYYCAQGGCGNYYRESWGQGTQVTVSS

SEQ ID NO: 4 - protein, 119 aa, synthetic construct
AVQLVESGGGLVQPGGSLRLSCAASGFAFRRYTMSWVRQAPGKGLEWVSSITDNGSITTYAESAQGRFTISRDNAKNTLYLQMNSLKPEDTAVYHCAQGGGSNYYRESWGQGTQVTVSS

SEQ ID NO: 5 - protein, 119 aa, synthetic construct
QVQLVESGGGLVQPGGSLRLSCAASGFSFRRYTMSWVRQAPGKGLEWVSAISDNGAITTYTESAQGRFTISRDNAKNTLYLQMNSLKPEDTAVYYCAQGGGSNYYRESWGQGTQVTVSS

SEQ ID NO: 6 - protein, 119 aa, synthetic construct
PVQLVESGGGLVQPGGSLRLSCAASGFSFRNYIMSWVRQAPGKGLEWLSSITNSGGITSYTESVKGRFTISRDNAKNTLYLQMNSLKPEDTAVYYCAQGGSDNYYRGSWGQGTQVTVSS

SEQ ID NO: 7 - protein, 121 aa, synthetic construct
PVQLVESGGNLVQPGGSLRLSCVAPGFTSRNSAMSWVRQAPGKGLEWVSTIYSGKSNTDYADSVKGRFTISTDNAKNTLYLHMNKLKSEDTAVYYCAKGAASDWYVPRDYWGQGTQVTVSS

SEQ ID NO: 8 - protein, 119 aa, synthetic construct
PVQLVESGGGLVQPGGSLRLSCAASGFTFRNYAMSWVRQAPGKGLEWVSTITHNGAITTYAESAQGRFTISRDNAKNTLYLQMNSLKPEDTAVYYCAQGGGSNYYRESWGQGTQVTVSS

SEQ ID NO: 9 - protein, 123 aa, synthetic construct
QLHFVESGGGLVQPGGSLRLSCAASGFTLDHYAMGWFRQAPGKEREGVSCISGGGSTRYADSAKGRFTISRDNAKNTVYLQMNSLTPEDTAVYYCAAARLLSRNCVPRDSGSWGQGTQVTVSS

SEQ ID NO: 10 - protein, 123 aa, synthetic construct
QVQLVESGGGLVQPGGSLRLSCVASGFTLDYFAIGWFRQAPGKEREGVSCISGGGSTRYADSVKGRFTISRDNAKNTVYLQMNSLTPEDTAVYYCAAARLLSRNCVPRDSGSWGQGTQVTVSS

SEQ ID NO: 11 - protein, 123 aa, synthetic construct
QLHFVESGGGLVQPGGSLRLSCAASGFTLDYYAIGWFRQAPGKEREGVSCISGGGSTRYADSVKGRFTISRDNAKNTVYLQMNSLTPEDTAVYYCAAARLLSTNCVPRDSGSWGQGTQVTVSS

SEQ ID NO: 12 - protein, 119 aa, synthetic construct
QLQLVESGGGLVQAGGSLRLSCAASGRIFSSYAMSWFRQAPGKEREFVATIGWIGENTYYADSVKGRFTISRDNAKNTLYLQMNSLKPEDTAVYYCAAGLPANRYYDYWGQGTQVTVSS

SEQ ID NO: 13 - protein, 127 aa, synthetic construct
EVQVVESGGGLVQPGGSLRLTCAASGFSVDDYGIGWFRQAPGREREGVACITPNGLMMNFANTVGSVAGRFSASRDSGENTVSLQMSSLKPEDTAIYFCAHSRDDSCRGSLSDYDDWGQGTQVTVSS

SEQ ID NO: 14 - protein, 5 aa, synthetic construct
SYTMS

SEQ ID NO: 15 - protein, 17 aa, synthetic construct
TITHNGAITTYAESAQG

SEQ ID NO: 16 - protein, 10 aa, synthetic construct
GGGSNYYRES

SEQ ID NO: 17 - protein, 5 aa, synthetic construct
RYTMS

SEQ ID NO: 18 - protein, 17 aa, synthetic construct
TITHNGSITTYAESAQG

SEQ ID NO: 19 - protein, 10 aa, synthetic construct
GGGSNYYRES

SEQ ID NO: 20 - protein, 5 aa, synthetic construct
NYMMN

SEQ ID NO: 21 - protein, 17 aa, synthetic construct
SITSAGDITSYTESVKG

SEQ ID NO: 22 - protein, 10 aa, synthetic construct
GGCGNYYRES

SEQ ID NO: 23 - protein, 5 aa, synthetic construct
RYTMS

SEQ ID NO: 24 - protein, 17 aa, synthetic construct
SITDNGSITTYAESAQG

SEQ ID NO: 25 - protein, 10 aa, synthetic construct
GGGSNYYRES

SEQ ID NO: 26 - protein, 5 aa, synthetic construct
RYTMS

SEQ ID NO: 27 - protein, 17 aa, synthetic construct
AISDNGAITTYTESAQG

SEQ ID NO: 28 - protein, 10 aa, synthetic construct
GGGSNYYRES

SEQ ID NO: 29 - protein, 5 aa, synthetic construct
NYIMS

SEQ ID NO: 30 - protein, 17 aa, synthetic construct
SITNSGGITSYTESVKG

SEQ ID NO: 31 - protein, 10 aa, synthetic construct
GGSDNYYRGS

SEQ ID NO: 32 - protein, 5 aa, synthetic construct
NSAMS

SEQ ID NO: 33 - protein, 17 aa, synthetic construct
TIYSGKSNTDYADSVKG

SEQ ID NO: 34 - protein, 12 aa, synthetic construct
GAASDWYVPRDY

SEQ ID NO: 35 - protein, 5 aa, synthetic construct
NYAMS

SEQ ID NO: 36 - protein, 17 aa, synthetic construct
TITHNGAITTYAESAQG

SEQ ID NO: 37 - protein, 10 aa, synthetic construct
GGGSNYYRES

SEQ ID NO: 38 - protein, 5 aa, synthetic construct
HYAMG

SEQ ID NO: 39 - protein, 16 aa, synthetic construct
CISGGGSTRYADSAKG

SEQ ID NO: 40 - protein, 15 aa, synthetic construct
ARLLSRNCVPRDSGS

SEQ ID NO: 41 - protein, 5 aa, synthetic construct
YFAIG

SEQ ID NO: 42 - protein, 16 aa, synthetic construct
CISGGGSTRYADSVKG

SEQ ID NO: 43 - protein, 15 aa, synthetic construct
ARLLSRNCVPRDSGS

SEQ ID NO: 44 - protein, 5 aa, synthetic construct
YYAIG

SEQ ID NO: 45 - protein, 16 aa, synthetic construct
CISGGGSTRYADSVKG

SEQ ID NO: 46 - protein, 15 aa, synthetic construct
ARLLSTNCVPRDSGS

SEQ ID NO: 47 - protein, 5 aa, synthetic construct
SYAMS

SEQ ID NO: 48 - protein, 17 aa, synthetic construct
TIGWIGENTYYADSVKG

SEQ ID NO: 49 - protein, 10 aa, synthetic construct
GLPANRYYDY

SEQ ID NO: 50 - protein, 5 aa, synthetic construct
DYGIG

SEQ ID NO: 51 - protein, 20 aa, synthetic construct
CITPNGLMMNFANTVGSVAG

SEQ ID NO: 52 - protein, 15 aa, synthetic construct
SRDDSCRGSLSDYDD

SEQ ID NO: 53 - protein, 119 aa, synthetic construct
EVQLVESGGGLVQPGGSLRLSCAASGFAFRSYTMSWVRQAPGKGLEWVSTITHNGAITTYAESAQGRFTISRDNSKNTLYLQMNSLRAEDTAVYYCAKGGGSNYYRESWGQGTQVTVSS

SEQ ID NO: 54 - protein, 119 aa, synthetic construct
EVQLVESGGGLVQPGGSLRLSCAASGFAFRSYTMSWVRQAPGKGLEWVSTITHNGAITTYAESAQGRFTISRDNSKNTLYLQMNSLRAEDTAVYYCAQGGGSNYYRESWGQGTQVTVSS

SEQ ID NO: 55 - protein, 119 aa, synthetic construct
EVQLVESGGGLVQPGGSLRLSCAASGFAFRSYTMSWVRQAPGKGLEWVSTITHNAAITTYAESAQGRFTISRDNSKNTLYLQMNSLRAEDTAVYYCAQGGGSNYYRESWGQGTQVTVSS

SEQ ID NO: 56 - protein, 119 aa, synthetic construct
EVQLVESGGGLVQPGGSLRLSCAASGFAFRSYTMSWVRQAPGKGLEWVSTITHQGAITTYAESAQGRFTISRDNSKNTLYLQMNSLRAEDTAVYYCAQGGGSNYYRESWGQGTQVTVSS

SEQ ID NO: 57 - protein, 119 aa, synthetic construct
EVQLVESGGGLVQPGGSLRLSCAASGFAFRSYTMSWVRQAPGKGLEWVSTITHNGAITTYAESAQGRFTISRDNSKNTLYLQMNSLRPEDTAVYYCAQGGGSNYYRESWGQGTQVTVSS

SEQ ID NO: 58 - protein, 119 aa, synthetic construct
EVQLVESGGGLVQPGGSLRLSCAASGFAFRSYTMSWVRQAPGKGLEWVSTITHNAAITTYAESAQGRFTISRDNSKNTLYLQMNSLRPEDTAVYYCAQGGGSNYYRESWGQGTQVTVSS

SEQ ID NO: 59 - protein, 119 aa, synthetic construct
EVQLVESGGGLVQPGGSLRLSCAASGFAFRSYTMSWVRQAPGKGLEWVSTITHQGAITTYAESAQGRFTISRDNSKNTLYLQMNSLRPEDTAVYYCAQGGGSNYYRESWGQGTQVTVSS

SEQ ID NO: 60 - protein, 119 aa, synthetic construct
PSQLVESGGGLVQPGGSLRLSCAASGFAFRSYTMSWVRQAPGKGLEWVSTITHNGAITTYAESAQGRFTISRDNSKNTLYLQMNSLRPEDTAVYYCAQGGGSNYYRESWGQGTQVTVSS

SEQ ID NO: 61 - protein, 119 aa, synthetic construct
PSQLVESGGGLVQPGGSLRLSCAASGFAFRSYTMSWVRQAPGKGLEWVSTITHNAAITTYAESAQGRFTISRDNSKNTLYLQMNSLRPEDTAVYYCAQGGGSNYYRESWGQGTQVTVSS

SEQ ID NO: 62 - protein, 119 aa, synthetic construct
PSQLVESGGGLVQPGGSLRLSCAASGFAFRSYTMSWVRQAPGKGLEWVSTITHQGAITTYAESAQGRFTISRDNSKNTLYLQMNSLRPEDTAVYYCAQGGGSNYYRESWGQGTQVTVSS

SEQ ID NO: 63 - protein, 17 aa, synthetic construct
TITHNAAITTYAESAQG

SEQ ID NO: 64 - protein, 17 aa, synthetic construct
TITHQGAITTYAESAQG

SEQ ID NO: 65 - protein, 119 aa, synthetic construct
EVQLVESGGGLVQPGGSLRLSCAASGFAFRRYTMSWVRQAPGKGLEWVSTITHNGSITTYAESAQGRFTISRDNSKNTLYLQMNSLRAEDTAVYYCAKGGGSNYYRESWGQGTQVTVSS

SEQ ID NO: 66 - protein, 119 aa, synthetic construct
EVQLVESGGGLVQPGGSLRLSCAASGFAFRRYTMSWVRQAPGKGLEWVSTITHNGSITTYAESAQGRFTISRDNSKNTLYLQMNSLRAEDTAVYYCAQGGGSNYYRESWGQGTQVTVSS

SEQ ID NO: 67 - protein, 119 aa, synthetic construct
EVQLVESGGGLVQPGGSLRLSCAASGFAFRRYTMSWVRQAPGKGLEWVSTITHNGSITTYAESAQGRFTISRDNSKNTLYLQMNSLRPEDTAVYYCAQGGGSNYYRESWGQGTQVTVSS

SEQ ID NO: 68 - protein, 119 aa, synthetic construct
QSQLVESGGGLVQPGGSLRLSCAASGFAFRRYTMSWVRQAPGKGLEWVSTITHNGSITTYAESAQGRFTISRDNSKNTLYLQMNSLRPEDTAVYYCAQGGGSNYYRESWGQGTQVTVSS

SEQ ID NO: 69 - protein, 123 aa, synthetic construct
QVQLVESGGGLVKPGGSLRLSCAASGFTFSYFAIGWIRQAPGKGLEWVSCISGGGSTRYADSVKGRFTISRDNAKNSLYLQMNSLRAEDTAVYYCARARLLSRNCVPRDSGSWGQGTLVTVSS

SEQ ID NO: 70 - protein, 123 aa, synthetic construct
QVQLVESGGGLVKPGGSLRLSCAASGFTFDYFAIGWFRQAPGKGREGVSCISGGGSTRYADSVKGRFTISRDNAKNSVYLQMNSLRPEDTAVYYCARARLLSRNCVPRDSGSWGQGTLVTVSS

SEQ ID NO: 71 - protein, 123 aa, synthetic construct
QVQLVESGGGLVKPGGSLRLSCAASGFTFDYFAIGWFRQAPGKELEGVSCISGGGSTRYADSVKGRFTISRDNAKNSVYLQMNSLRPEDTAVYYCARARLLSRNCVPRDSGSWGQGTLVTVSS

SEQ ID NO: 72 - protein, 123 aa, synthetic construct
QVQLVESGGGLVKPGGSLRLSCAASGFTFDYFAIGWFRQAPGKELEGVSCISGGGSTRYADSVKGRFTISRDNAKNSVYLQMNSLRPEDTAVYYCAAARLLSRNCVPRDSGSWGQGTLVTVSS

SEQ ID NO: 73 - protein, 115 aa, synthetic construct
EVQLQQSGPELVKPGASVKISCKASGYAFSSSWMNWVKQRPGKGLEWIGRIYREDGHTNYNGKFKGKATLTADKSSTTAYMQLTSLTSEDSAVYFCANGGAMDYWGQGTSVTVSS

SEQ ID NO: 74 - protein, 107 aa, synthetic construct
DIQMTQSTSSLSASLGDRVTISCRASQDISNYLNWYQQKPDGTVKLLIYYTSRLHSGVPSRFSGSGSGTDYSLTISNLEQEDIATYFCQQDNTLPWTFGGGTKLEIK

SEQ ID NO: 75 - protein, 6 aa, synthetic construct
SSSWMN

SEQ ID NO: 76 - protein, 17 aa, synthetic construct
RIYREDGHTNYNGKFKG

SEQ ID NO: 77 - protein, 6 aa, synthetic construct
GGAMDY

SEQ ID NO: 78 - protein, 11 aa, synthetic construct
RASQDISNYLN

SEQ ID NO: 79 - protein, 7 aa, synthetic construct
YTSRLHS

SEQ ID NO: 80 - protein, 9 aa, synthetic construct
QQDNTLPWT

SEQ ID NO: 81 - protein, 115 aa, synthetic construct
QVQLVQSGAEVKKPGSSVKVSCKASGYAFSSSWMNWVRQAPGQGLEWMGRIYREDGHTNYNGKFKGRVTITADKSTTTAYMELSSLRSEDTAVYYCANGGAMDYWGQGTLVTVSS

SEQ ID NO: 82 - protein, 115 aa, synthetic construct
QVQLVQSGAEVKKPGSSVKVSCKASGGTFSSSWMNWVRQAPGQGLEWMGRIYREDGHTNYNGKFKGRVTITADKSTSTAYMELSSLRSEDTAVYYCANGGAMDYWGQGTLVTVSS

SEQ ID NO: 83 - protein, 115 aa, synthetic construct
QVQLVQSGAEVKKPGASVKVSCKASGYAFSSSWMNWVRQAPGQGLEWMGRIYREDGHTNYNGKFKGRVTMTADKSITTAYMELSRLRSDDTAVYYCANGGAMDYWGQGTLVTVSS

SEQ ID NO: 84 - protein, 115 aa, synthetic construct
QVQLVQSGAEVKKPGASVKVSCKASGYTFSSSWMNWVRQAPGQGLEWMGRIYREDGHTNYNGKFKGRVTMTADKSITTAYMELSRLRSDDTAVYYCANGGAMDYWGQGTLVTVSS

SEQ ID NO: 85 - protein, 115 aa, synthetic construct
QVQLVQSGAEVKKPGASVKVSCKASGYAFSSSWMNWVRQAPGQGLEWMGRIYREDGHTNYNGKFKGRVTMTADKSTTTAYMELSSLRSEDTAVYYCANGGAMDYWGQGTLVTVSS

SEQ ID NO: 86 - protein, 115 aa, synthetic construct
QVQLVQSGAEVKKPGASVKVSCKASGYTFSSSWMNWVRQAPGQGLEWMGRIYREDGHTNYNGKFKGRVTMTADKSTTTGYMELSSLRSEDTAVYYCANGGAMDYWGQGTLVTVSS

SEQ ID NO: 87 - protein, 115 aa, synthetic construct
QVQLVQSGAEVKKPGASVKVSCKASGYAFSSSWMNWVRQAPGQRLEWMGRIYREDGHTNYNGKFKGRVTITADKSATTAYMELSSLRSEDTAVYYCANGGAMDYWGQGTLVTVSS

SEQ ID NO: 88 - protein, 115 aa, synthetic construct
QVQLVQSGAEVKKPGASVKVSCKASGYTFSSSWMNWVRQAPGQRLEWMGRIYREDGHTNYNGKFKGRVTITADKSATTAYMELSSLRSEDTAVYYCANGGAMDYWGQGTLVTVSS

SEQ ID NO: 89 - protein, 115 aa, synthetic construct
QVQLVQSGAEVKKPGASVKVSCKASGYAFSSSWMNWVRQAPGQGLEWMGRIYREDGHTNYNGKFKGRVTMTADKSTTTAYMELRSLRSDDTAVYYCANGGAMDYWGQGTLVTVSS

SEQ ID NO: 90 - protein, 115 aa, synthetic construct
QVQLVQSGAEVKKPGASVKVSCKASGYTFSSSWMNWVRQAPGQGLEWMGRIYREDGHTNYNGKFKGRVTMTADKSTSTAYMELRSLRSDDTAVYYCANGGAMDYWGQGTLVTVSS

SEQ ID NO: 91 - protein, 107 aa, synthetic construct
DIQMTQSPSSLSASVGDRVTITCRASQDISNYLNWYQQKPGKAPKLLIYYTSRLHSGVPSRFSGSGSGTDYTFTISSLQPEDIATYFCQQDNTLPWTFGGGTKVEIK

SEQ ID NO: 92 - protein, 107 aa, synthetic construct
DIQMTQSPSSLSASVGDRVTITCRASQDISNYLNWYQQKPGKAPKLLIYYTSRLHSGVPSRFSGSGSGTDYTFTISSLQPEDIATYYCQQDNTLPWTFGGGTKVEIK

SEQ ID NO: 93 - protein, 107 aa, synthetic construct
DIQMTQSPSSLSASVGDRVTITCRASQDISNYLNWYQQKPGKAPKLLIYYTSRLHSGVPSRFSGSGSGTDYTLTISSLQPEDIATYFCQQDNTLPWTFGGGTKVEIK

SEQ ID NO: 94 - protein, 107 aa, synthetic construct
DIQMTQSPSSLSASVGDRVTITCRASQDISNYLNWYQQKPGKAPKLLIYYTSRLHSGVPSRFSGSGSGTDYTLTISSLQPEDFATYFCQQDNTLPWTFGGGTKVEIK

SEQ ID NO: 95 - protein, 107 aa, synthetic construct
EIVMTQSPATLSLSPGERATLSCRASQDISNYLNWYQQKPGQAPRLLIYYTSRLHSGIPARFSGSGSGTDYTLTISSLEPEDIAVYFCQQDNTLPWTFGGGTKVEIK

SEQ ID NO: 96 - protein, 107 aa, synthetic construct
EIVMTQSPATLSLSPGERATLSCRASQDISNYLNWYQQKPGQAPRLLIYYTSRLHSGIPARFSGSGSGTDYTLTISSLEPEDFAVYYCQQDNTLPWTFGGGTKVEIK

SEQ ID NO: 97 - protein, 107 aa, synthetic construct
DIVMTQSPDSLAVSLGERATINCRASQDISNYLNWYQQKPGQPPKLLIYYTSRLHSGVPDRFSGSGSGTDYTLTISSLQAEDIAVYFCQQDNTLPWTFGGGTKVEIK

SEQ ID NO: 98 - protein, 107 aa, synthetic construct
DIVMTQSPDSLAVSLGERATINCRASQDISNYLNWYQQKPGQPPKLLIYYTSRLHSGVPDRFSGSGSGTDYTLTISSLQAEDIAVYYCQQDNTLPWTFGGGTKVEIK

SEQ ID NO: 99 - protein, 107 aa, synthetic construct
DIQMTQSPSTLSASVGDRVTITCRASQDISNYLNWYQQKPGKAPKLLIYYTSRLHSGVPSRFSGSGSGTEYTLTISSLQPDDIATYFCQQDNTLPWTFGGGTKVEIK

SEQ ID NO: 100 - protein, 107 aa, synthetic construct
DIQMTQSPSTLSASVGDRVTITCRASQDISNYLNWYQQKPGKAPKLLIYYTSRLHSGVPSRFSGSGSGTEYTLTISSLQPDDFATYFCQQDNTLPWTFGGGTKVEIK

SEQ ID NO: 101 - protein, 122 aa, synthetic construct
EVKLVESGGGLVQPGGSLSLSCAASGFTFTDYYMSWVRQPPGKALEWLGFIRNKGNGYTTENSASVKGRFTISRDNSQSILYLQMNALRAEDSATYYCARYRGNPHYAMDYWGQGTSVTVSS

SEQ ID NO: 102 - protein, 111 aa, synthetic construct
DIVLTQFPASLAVSLGQRATISCRASESVEFYGTSFLQWYQQKPGQPPKLLIYGASNVESGVPARFSGSGSGTDFSLSIHPVEEDDIAMYFCQQSRKVPSTFGGGTKLEIK

SEQ ID NO: 103 - protein, 6 aa, synthetic construct
TDYYMS

SEQ ID NO: 104 - protein, 19 aa, synthetic construct
FIRNKGNGYTTENSASVKG

SEQ ID NO: 105 - protein, 11 aa, synthetic construct
YRGNPHYAMDY

SEQ ID NO: 106 - protein, 15 aa, synthetic construct
RASESVEFYGTSFLQ

SEQ ID NO: 107 - protein, 7 aa, synthetic construct
GASNVES

SEQ ID NO: 108 - protein, 9 aa, synthetic construct
QQSRKVPST

SEQ ID NO: 109 - protein, 122 aa, synthetic construct
EVKLVESGGGLVQPGGSLSLSCAASGFTFTDYYMSWVRQPPGKALEWLGFIRNKGNAYTTENSASVKGRFTISRDNSQSILYLQMNALRAEDSATYYCARYRGNPHYAMDYWGQGTSVTVSS

SEQ ID NO: 110 - protein, 122 aa, synthetic construct
EVQLVESGGGLVQPGGSLRLSCAASGFTFTDYYMSWVRQAPGKGLEWVGFIRNKGNGYTTENSASVKGRFTISRDNSKSTLYLQMNSLRAEDTAVYYCARYRGNPHYAMDYWGQGTLVTVSS

SEQ ID NO: 111 - protein, 122 aa, synthetic construct
EVQLVESGGGLIQPGGSLRLSCAASGFTFTDYYMSWVRQPPGKGLEWVGFIRNKGNGYTTENSASVKGRFTISRDNSKSTLYLQMNSLRAEDTAVYYCARYRGNPHYAMDYWGQGTLVTVSS

SEQ ID NO: 112 - protein, 122 aa, synthetic construct
QVQLVESGGGVVQPGGSLRLSCAASGFTFTDYYMSWVRQAPGKGLEWVGFIRNKGNGYTTENSASVKGRFTISRDNSKSTLYLQMNSLRAEDTAVYYCARYRGNPHYAMDYWGQGTLVTVSS

SEQ ID NO: 113 - protein, 122 aa, synthetic construct
EVQLVESGGGLVQPGGSLRLSCAASGFTFTDYYMSWVRQAPGKGLEWVGFIRNKGNGYTTENSASVKGRFTISRDNSKSSLYLQMNSLKTEDTAVYYCARYRGNPHYAMDYWGQGTLVTVSS

SEQ ID NO: 114 - protein, 122 aa, synthetic construct
EVQLVESGGGLVQPGRSLRLSCTASGFTFTDYYMSWVRQAPGKGLEWVGFIRNKGNGYTTENSASVKGRFTISRDNSKSILYLQMNSLKTEDTAVYYCARYRGNPHYAMDYWGQGTLVTVSS

SEQ ID NO: 115 - protein, 122 aa, synthetic construct
EVQLVESGGGLVQPGGSLRLSCAASGFTFTDYYMSWVRQAPGKGLEWVGFIRNKGNGYTTENSASVKGRFTISRDNAKSSLYLQMNSLRAEDTAVYYCARYRGNPHYAMDYWGQGTLVTVSS

SEQ ID NO: 116 - protein, 111 aa, synthetic construct
DIVLTQSPDSLAVSLGERATINCRASESVEFYGTSFLQWYQQKPGQPPKLLIYGASNVESGVPDRFSGSGSGTDFTLTISSLQAEDIAVYFCQQSRKVPSTFGGGTKVEIK

SEQ ID NO: 117 - protein, 111 aa, synthetic construct
EIVLTQSPATLSVSPGERATLSCRASESVEFYGTSFLQWYQQKPGQAPRLLIYGASNVESGIPARFSGSGSGTEFTLTISSLQSEDIAVYFCQQSRKVPSTFGGGTKVEIK

SEQ ID NO: 118 - protein, 111 aa, synthetic construct
DIQLTQSPSSLSASVGDRVTITCRASESVEFYGTSFLQWYQQKPGKAPKLLIYGASNVESGVPSRFSGSGSGTDFTLTISSLQPEDIATYFCQQSRKVPSTFGGGTKVEIK

SEQ ID NO: 119 - protein, 111 aa, synthetic construct
DIVLTQSPLSLPVTPGEPASISCRASESVEFYGTSFLQWYLQKPGQSPQLLIYGASNVESGVPDRFSGSGSGTDFTLKISRVEAEDIGVYFCQQSRKVPSTFGGGTKVEIK

SEQ ID NO: 120 - protein, 111 aa, synthetic construct
EIVLTQSPGTLSLSPGERATLSCRASESVEFYGTSFLQWYQQKPGQAPRLLIYGASNVESGIPDRFSGSGSGTDFTLTISRLEPEDIAVYFCQQSRKVPSTFGGGTKVEIK

SEQ ID NO: 121 - protein, 120 aa, synthetic construct
DVQLVESGGGLVQPGGSRKLSCTASGFTFSNFGMHWVRQAPETGLEWVAYISSGSSTFYYADSVKGRFTISRDNPKNTLFLQMTSLRSEDTAMYFCARSPAYYRYGLDYWGQGTTLTVSS

SEQ ID NO: 122 - protein, 111 aa, synthetic construct
DIVLTQSPASLAVSLGQRATISCRASQSVSSSTFSYMHWYQQKPGQPPKLLIKSSSNLESGVPARFSGSGSGTDFTLNIHPVEEEDTATYYCQHSWEIPYTFGGGTKLEIK

SEQ ID NO: 123 - protein, 5 aa, synthetic construct
NFGMH

SEQ ID NO: 124 - protein, 17 aa, synthetic construct
YISSGSSTFYYADSVKG

SEQ ID NO: 125 - protein, 11 aa, synthetic construct
SPAYYRYGLDY

SEQ ID NO: 126 - protein, 15 aa, synthetic construct
RASQSVSSSTFSYMH

SEQ ID NO: 127 - protein, 7 aa, synthetic construct
SSSNLES

SEQ ID NO: 128 - protein, 9 aa, synthetic construct
QHSWEIPYT

SEQ ID NO: 129 - protein, 120 aa, synthetic construct
DVQLVESGGGLVQPGGSRKLSCTASGFTFSNFGMHWVRQAPETGLEWVAYISSGSSTFYYADAVKGRFTISRDNPKNTLFLQMTSLRSEDTAMYFCARSPAYYRYGLDYWGQGTTLTVSS

SEQ ID NO: 130 - protein, 120 aa, synthetic construct
EVQLVESGGGVVQPGRSLRLSCAASGFTFSNFGMHWVRQAPGKGLEWVAYISSGSSTFYYADSVKGRFTISRDNSKNTLYLQMNSLRAEDTAVYYCARSPAYYRYGLDYWGQGTTVTVSS

SEQ ID NO: 131 - protein, 120 aa, synthetic construct
EVQLVESGGGVVQPGRSLRLSCAASGFTFSNFGMHWVRQAPGKGLEWVAYISSGSSTFYYADSVKGRFTISRDNSKNTLYLQMNSLRAEDTAVYFCARSPAYYRYGLDYWGQGTTVTVSS

SEQ ID NO: 132 - protein, 111 aa, synthetic construct
DIVMTQTPLSLSVTPGQPASISCRASQSVSSSTFSYMHWYQQKPGQPPQLLIYSSSNLESGVPDRFSGSGSGTDFTLKISRVEAEDVGVYYCQHSWEIPYTFGGGTKVEIK

SEQ ID NO: 133 - protein, 111 aa, synthetic construct
DIVMTQTPLSLSVTPGQPASISCRASQSVSSSTFSYMHWYQQKPGQPPQLLIKSSSNLESGVPDRFSGSGSGTDFTLKISRVEAEDVGVYYCQHSWEIPYTFGGGTKVEIK

SEQ ID NO: 134 - protein, 111 aa, synthetic construct
DIVLTQTPLSLSVTPGQPASISCRASQSVSSSTFSYMHWYQQKPGQPPQLLIKSSSNLESGVPDRFSGSGSGTDFTLKISRVEAEDVGVYYCQHSWEIPYTFGGGTKVEIK

SEQ ID NO: 135 - protein, 111 aa, synthetic construct
DIVMTQSPDSLAVSLGERATINCRASQSVSSSTFSYMHWYQQKPGQPPKLLIYSSSNLESGVPDRFSGSGSGTDFTLTISSLQAEDVAVYYCQHSWEIPYTFGGGTKVEIK

SEQ ID NO: 136 - protein, 111 aa, synthetic construct
DIVMTQSPDSLAVSLGERATINCRASQSVSSSTFSYMHWYQQKPGQPPKLLIKSSSNLESGVPDRFSGSGSGTDFTLTISSLQAEDVAVYYCQHSWEIPYTFGGGTKVEIK

SEQ ID NO: 137 - protein, 111 aa, synthetic construct
DIVLTQSPDSLAVSLGERATINCRASQSVSSSTFSYMHWYQQKPGQPPKLLIKSSSNLESGVPDRFSGSGSGTDFTLTISSLQAEDVAVYYCQHSWEIPYTFGGGTKVEIK

SEQ ID NO: 138 - protein, 118 aa, synthetic construct
DVQLHESGPGLVKPSQSLSLTCSVTGYSITNNYYWNWIRQFPGNNLEWMGYITYDGSNNYNPSLKNRISVTRDTSKNQFFLKLSSVTTEDTATYYCTRGGGQLRFDYWGQGTTLTVSS

SEQ ID NO: 139 - protein, 107 aa, synthetic construct
SIVMTQTPKFLIVSAGDRVTITCKASQSVSNEVTWYQQKPGQSPKMLIYYASNRYTGVPDRFTGSGYGTDFTFTINTVQAEDLAIYFCQQDYSSPWTFGGGTKLEIK

SEQ ID NO: 140 - protein, 6 aa, synthetic construct
NNYYWN

SEQ ID NO: 141 - protein, 16 aa, synthetic construct
YITYDGSNNYNPSLKN

SEQ ID NO: 142 - protein, 9 aa, synthetic construct
GGGQLRFDY

SEQ ID NO: 143 - protein, 11 aa, synthetic construct
KASQSVSNEVT

SEQ ID NO: 144 - protein, 7 aa, synthetic construct
YASNRYT

SEQ ID NO: 145 - protein, 9 aa, synthetic construct
QQDYSSPWT

SEQ ID NO: 146 - protein, 118 aa, synthetic construct
DVQLHESGPGLVKPSQSLSLTCSVTGYSITNNYYWNWIRQFPGNNLEWMGYITYDASNNYNPSLKNRISVTRDTSKNQFFLKLSSVTTEDTATYYCTRGGGQLRFDYWGQGTTLTVSS

SEQ ID NO: 147 - protein, 118 aa, synthetic construct
EVQLQESGPGLVKPSDTLSLTCAVSGYSISNNYYWNWIRQPPGKGLEWIGYITYDGSNNYNPSLKNRVTMSVDTSKNQFSLKLSSVTAVDTAVYYCARGGGQLRFDYWGQGTTVTVSS

SEQ ID NO: 148 - protein, 118 aa, synthetic construct
EVQLQESGPGLVKPSDTLSLTCAVSGYSISNNYYWNWIRQPPGKGLEWIGYITYDGSNNYNPSLKNRVTMSRDTSKNQFSLKLSSVTAVDTAVYYCARGGGQLRFDYWGQGTTVTVSS

SEQ ID NO: 149 - protein, 118 aa, synthetic construct
EVQLQESGPGLVKPSDTLSLTCAVSGYSISNNYYWNWIRQPPGKGLEWIGYITYDGSNNYNPSLKNRITMSRDTSKNQFSLKLSSVTAVDTAVYYCARGGGQLRFDYWGQGTTVTVSS

SEQ ID NO: 150 - protein, 118 aa, synthetic construct
EVQLQESGPGLVKPSDTLSLTCAVSGYSISNNYYWNWIRQPPGKGLEWMGYITYDGSNNYNPSLKNRITVSRDTSKNQFSLKLSSVTAVDTAVYYCARGGGQLRFDYWGQGTTVTVSS

SEQ ID NO: 151 - protein, 118 aa, synthetic construct
EVQLQESGPGLVKPSDTLSLTCAVSGYSITNNYYWNWIRQPPGKGLEWMGYITYDGSNNYNPSLKNRITVSRDTSKNQFSLKLSSVTAVDTAVYYCTRGGGQLRFDYWGQGTTVTVSS

SEQ ID NO: 152 - protein, 107 aa, synthetic construct
DIQMTQSPSSLSASVGDRVTITCKASQSVSNEVTWYQQKPGKAPKLLIYYASNRYTGVPSRFSGSGSGTDFTFTISSLQPEDIATYYCQQDYSSPWTFGGGTKVEIK

SEQ ID NO: 153 - protein, 107 aa, synthetic construct
DIQMTQSPSSLSASVGDRVTITCKASQSVSNEVTWYQQKPGKAPKLLIYYASNRYTGVPDRFSGSGSGTDFTFTISSLQPEDIATYYCQQDYSSPWTFGGGTKVEIK

SEQ ID NO: 154 - protein, 107 aa, synthetic construct
DIQMTQSPSSLSASVGDRVTITCKASQSVSNEVTWYQQKPGKAPKMLIYYASNRYTGVPDRFSGSGSGTDFTFTISSLQPEDIATYYCQQDYSSPWTFGGGTKVEIK

SEQ ID NO: 155 - protein, 107 aa, synthetic construct
DIQMTQSPSSLSASVGDRVTITCKASQSVSNEVTWYQQKPGKAPKMLIYYASNRYTGVPDRFSGSGYGTDFTFTISSLQPEDIATYYCQQDYSSPWTFGGGTKVEIK

SEQ ID NO: 156 - protein, 107 aa, synthetic construct
DIQMTQSPSSLSASVGDRVTITCKASQSVSNEVTWYQQKPGKSPKMLIYYASNRYTGVPDRFSGSGYGTDFTFTISSLQPEDIATYFCQQDYSSPWTFGGGTKVEIK

SEQ ID NO: 157 - protein, 107 aa, synthetic construct
SIVMTQSPSSLSASVGDRVTITCKASQSVSNEVTWYQQKPGKSPKMLIYYASNRYTGVPDRFSGSGYGTDFTFTISSLQPEDIATYFCQQDYSSPWTFGGGTKVEIK

SEQ ID NO: 158 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 20 aa, synthetic construct
GGGGSGGGGSGGGGSGGGGS

SEQ ID NO: 160 - protein, 107 aa, synthetic construct [sequence omitted]

SEQ ID NO: 161 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 162 - protein, 232 aa, synthetic construct
EPKSADKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 15 aa, synthetic construct
GGGGSGGGGSGGGGS

SEQ ID NO: 164 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 165 - protein, 232 aa, synthetic construct
EPKSADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK